(19) United States
(12) Patent Application Publication
(10) Pub. No.: US 2024/0424135 A1
(43) Pub. Date: Dec. 26, 2024

(54) Compositions For and Methods of Improving Viral Vectors

(71) Applicant: Duke University, Durham, NC (US)

(72) Inventors: Boris Kantor, Durham, NC (US); Ornit Chiba-Falek, Durham, NC (US); Malik Moncalvo, Durham, NC (US)

(21) Appl. No.: 18/578,772

(22) PCT Filed: Jul. 13, 2022
(86) PCT No.: PCT/US22/73682

§ 371 Date: Jan. 12, 2024

Related U.S. Application Data
(60) Provisional application No. 63/221,167, filed on Jul. 13, 2021.

(51) Int. Cl. A61K 48/00; C12N 9/22; C12N 15/11; C12N 15/113; C12N 15/86

(52) U.S. Cl. CPC A61K 48/005; C12N 9/22; C12N 15/111; C12N 15/1137; C12N 15/86; C12N 2310/14; C12N 2310/20; C12N 2740/15043; C12N 2750/14143; C12Y 305/01098

(57) ABSTRACT

Disclosed herein are viral vectors for use in methods of developing HDAC-depleted cells. Disclosed herein are methods of increasing packaging capacity, increasing the titer, increasing the expression capacity, and decreasing the immunogenicity and/or toxicity of an optimized viral vector generated in HDAC-depleted cells.

### I. CROSS-REFERENCE TO RELATED APPLICATIONS

This application claims the benefit of priority to U.S. Provisional Patent Application No. 63/221,167 filed 13 Jul. 2021, which is incorporated by reference herein in its entirety.

### II. REFERENCE TO THE SEQUENCE LISTING

The Sequence Listing submitted 13 Jul. 2022 as an .xml file named “POL 22-2050-WO -109726-733539”, created on 13 Jul. 2022 and having a size of 173 kilobytes is hereby incorporated by reference pursuant to 37 C.F.R. § 1.52(e)(5).

### III. BACKGROUND

The CRISPR/Cas9 systems have revolutionized the field of genome editing by providing unprecedented control over gene sequences and gene expression in many species, including humans. But currently there is no ideal vector platform for delivery of therapeutic transgenes including those based on the CRISPR/Cas-editors.

For example, while adeno-associated virus (AAV) vectors are the most common delivery systems used for gene therapy applications, AAV vectors have strict packaging size limits (up to 4.7 kb), which is prohibitive for most all-in-one CRISPR/Cas-based systems. Lentiviral vectors (LVs) are one of the primary delivery platforms for the CRISPR/Cas9 system due to their ability to accommodate large DNA payloads (up to 11 kb) and sustain robust expression in a wide range of dividing and non-dividing cells. However, the employment of LVs in clinical trials has been hampered by a relatively high risk of insertional mutagenesis. (Ortinski P I, et al. (2017) Mol Ther Methods Clin Dev. 5:153-164). Furthermore, long-term expression of LV-delivering Cas9/gRNA may lead to undesirable off-target effects characterized by non-specific RNA-DNA interactions and off-target DNA perturbations (Ortinski P I, et al. (2017) Mol Ther Methods Clin Dev. 5:153-164). Finally, the advantages of LVs used for CRISPR/Cas9 delivery are counterbalanced by the low titers associated with their production as the viral genomes are packaged into heterochromatin structures largely inaccessible for general transcription machinery.

Thus, what is needed is a gene delivery system is a vector that combines the advantages of both AAV and LV vectors while minimizing the known limitations of these vectors. There is a major unmet need for vectors that demonstrate episomal expression, have high production yields, have high packaging capacity, and generate strong transgene expression.

### IV. BRIEF DESCRIPTION OF THE FIGURES

FIG. 1A-FIG. 1C detail vector production. FIG. 1A shows a schematic map of the lentiviral vector cassette plasmid. The version on the top lacks an Sp1 binding site while the version on the bottom contains two Sp1 binding sites. Other regulatory elements include primer binding site (PBS), splice donor (SD) and splice acceptor (SA), central polypurine tract (cPPT) and polypurine tract (PPT), Rev Response element (RRE), Woodchuck Hepatitis Virus Posttranscriptional Regulatory Element (WPRE), and the retroviral vector packaging element (the psi (ψ) signal). A human Cytomegalovirus (hCMV) promoter, a core-elongation factor 1a promoter (EFS), and a human U6 promoter are also included. The self-inactivated vector (SIN) cassette plasmid carries a deletion (−18 bps to −418 bps) in the U3 region of 3′-LTR (DU3). A polylinker site contains a pair of BsmBI sites and a unique BsrGI site used for cloning of sgRNA and for its verification, respectively. FIG. 1B presents production titers of integrating (ICLV) and non-integrating (IDLV) viral particles+/−Sp1 sites. The results are recorded in copy number per milliliter, equating 1 ng of p24gag to 1×10⁴ particles. FIG. 1C shows the overall ICLV production titer determined by counting puromycin-resistant colonies. The results are shown as the ratio between number of colonies obtained from the vector with and without Sp1. The bar graph data represents mean±SD from triplicate experiments.

FIG. 2 illustrates the efficiency of CRISPR/Cas9 mediated knockout. The level of eGFP depletion was evaluated for sgRNAs delivered by ICLV and IDLV by fluorescence-activated cell sorting assay 7 days, 14 days, and 21 days pt. Naïve HEK 293T-eGFP cells (not transduced with GFP virus) and non-sgRNA-expressed cells (no-sgRNA) served as controls. A percentage of GFP-positive cells remaining after transduction was recorded. The y axis represents side scatter while the x axis represents MFI.

FIG. 3A illustrates on-target evaluation of DNA cleavages of CRISPR/Cas9 delivered by IDLV and ICLV. The evaluation was done at 7 days pt using GFP-positive cells transduced with IDLV-sgRNA1/Cas9 and ICLV-sgRNA1/Cas9 at varying MOIs. FIG. 3B shows the use of the Sanger method to assess on target InDels of the samples transduced with either ICLV-vectors or IDLV-vectors. The rate of on-target mutations was determined at day 7. FIG. 3C illustrates the formation of Indels induced by ICLV-CRISPR/Cas9 (dark bars) and ICLV-CRISPR/Cas9 (light bars). The InDels were calculated as the ratio (in percentages) of reads with mutated sequences and total reads.

FIG. 4A show the use of western blot analysis to evaluate the efficacy of the IDLV-CRIPSR/Cas9 system to deplete the GABAA receptor α2 subunit in vivo (rats). Two control (lanes 1 and 2) and two IDLV-α2/Cas9 injected (lanes 3 and 4) animals (rats) were used. Tubulin (DM1A) antibody (˜40 kDa) was used as a loading control. FIG. 4B illustrates use of the mIPSCs method in NAc slices from control and IDLV-α2/Cas9-injected animals. Notice the similarity in event frequency at the top part of the figure (part i). mIPSC averages from three different neurons (black traces) illustrate variability of mIPSC duration in slices from IDLV-α2/Cas9 animals (rats) in the bottom part of the figure (part ii). For ease of comparison, each trace was overlaid onto a mIPSCs average from a single control neuron (gray trace). FIG. 4C illustrates distribution of mIPSC decay times from all recorded neurons. The horizontal black bars are centered at the mean values for each group. FIG. 4D illustrates K-means cluster analyses identify center values for slow, medium, and fast mIPSC groups of MSNs (n=1, 5, and 5 IDLV-α2/Cas9 cells in clusters 1, 2, and 3, respectively, while n=3, 2, and 2 control cells in clusters 1, 2, and 3, respectively). FIG. 4E shows the variability of mIPSC amplitudes was similar between cells in IDLV-α2/Cas9 and control groups. The horizontal black bars are centered at the mean values for each group.

FIG. 5A-FIG. 5B show the validation of histone deacetylases (HDACs) knockdown in HEK 293T cells. HEK 293T cells were transduced with LVs harboring shRNA to the HDACs. Here the representative image highlights the knockdown achieved using shRNA-to-HDAC2 and shRNA-to-HDAC8. In FIG. 5A, the upper graph shows the mRNA levels of HDAC8 measured in naïve HEK 293T cells (black bar), in those cells transduced with HDAC2-shRNA (white bar), and in those cells transduced with HDAC8-shRNA (grey bar) using qRT analysis. In FIG. 5A, the lower panel shows the mRNA levels of HDAC2 measured in naïve HEK 293T cells (black bar), in those cells transduced with HDAC2-shRNA (white bar), and in those cells transduced with HDAC8-shRNA (grey bar) using qRT analysis. FIG. 5B shows the protein levels of HDAC8 and HDAC2 evaluated by WB performed on the cell lysates prepared from HDAC2-transduced cells and HDAC8-transduced cells. Human actin was used as a loading control. As shown, specific and robust knockdown of HDAC8 was achieved in HDAC8-targeted cells, while the levels of HDAC2 protein were efficiently reduced in HDAC2 KD cell line.

FIG. 6 shows the efficient production of IDLV vectors generated in the naïve cells, cells carrying shRNA to HDAC1, HDAC2, HDAC3, HDAC4, and HDAC8, and cells treated with sodium butyrate (Bu) at 2.5 mM. The plot reports the quantity measurement of the production using p24gag ELISA as described (Tagliafierro L, et al. (2019) J Vis Exp. (145):10.3791/59241). The further normalization was made to apply the equal viral load for transduction into HEK 293T cells.

FIG. 7 reports the visualization of the GFP expression in the cells transduced with shRNA viruses harbored hair-pin to each of HDAC1, HDAC2, HDAC3, HDAC4, and HDAC8 as well as the cells treated with sodium butyrate (Bu) at 2.5 mM. To normalize the viral load, equal concentration of p24 content were used. The GFP expression was assessed using fluorescent microscopy method as described (Tagliafierro L, et al. (2019) J Vis Exp. (145):10.3791/59241).

FIG. 8 shows the map of SHC002, which is a non-mammalian shRNA control plasmid. SHC002 is a negative control that contains a sequence that does not target any known mammalian genes but engages with RISC. This non-mammalian control served as a negative reference for HDAC knockdowns. In FIG. 8, U6 mean U6 Promoter, cppt means central polypurine tract, hPGK means human phosphoglycerate kinase eukaryotic promoter, puroR means puromycin resistance gene for mammalian selection, SIN/3 c means LTR 3 c means self-inactivating long terminal repeat, f1 on means f1 origin of replication, ampR means ampicillin resistance gene for bacterial selection, pUC on means pUC origin of replication, 5 c LTR means 5 c long terminal repeat, Psi means RNA packaging signal, and RRE means rev response element.

FIG. 9A shows an improved lentiviral vector backbone harboring two Sp1 sites and a gRNA to GFP while FIG. 9B shows an improved lentiviral vector backbone harboring two Sp1 sites without a gRNA.

### V. BRIEF SUMMARY

Disclosed herein are compositions for use in performing one or more disclosed methods.

Disclosed herein is an isolated nucleic acid molecule comprising a nucleic acid sequence encoding (i) a Cas endonuclease, (ii) at least one guide RNA, and (iii) at least one transgene.

Disclosed herein is an isolated nucleic acid molecule comprising a nucleic acid sequence encoding (i) a fusion product, and (ii) at least one guide RNA.

Disclosed herein is an isolated nucleic acid molecule comprising a nucleic acid sequence encoding (i) a Cas endonuclease, (ii) at least one guide RNA, (iii) at least one transgene, and (iv) shRNA targeting a histone deacetylase (HDAC).

Disclosed herein is an isolated nucleic acid molecule comprising a nucleic acid sequence encoding (i) a fusion product, (ii) at least one guide RNA, and (iii) and shRNA targeting a histone deacetylase (HDAC).

Disclosed herein is an isolated nucleic acid molecule comprising a nucleic acid sequence encoding a shRNA targeting a histone deacetylase (HDAC). Disclosed herein is an isolated nucleic acid molecule comprising a nucleic acid sequence encoding one or more shRNAs targeting a histone deacetylase (HDAC). Disclosed herein is an isolated nucleic acid molecule comprising a nucleic acid sequence encoding one or more shRNAs targeting one or more histone deacetylases (HDAC).

Disclosed herein is a viral vector comprising a disclosed isolated nucleic acid. Disclosed herein is a viral vector comprising an isolated nucleic acid molecule comprising a nucleic acid sequence encoding (i) a Cas endonuclease, (ii) at least one guide RNA, and (iii) at least one transgene. Disclosed herein is a viral vector comprising an isolated nucleic acid molecule comprising a nucleic acid sequence encoding (i) a fusion product, and (ii) at least one guide RNA.

Disclosed herein is a viral vector comprising the sequence set forth in any one of SEQ ID NO:26-SEQ ID NO:31.

Disclosed herein is a viral vector comprising the sequence set forth in SEQ ID NO:26 and comprising a disclosed transgene. Disclosed herein is a viral vector comprising the sequence set forth in SEQ ID NO:27 and comprising a disclosed transgene. Disclosed herein is a viral vector comprising the sequence set forth in SEQ ID NO:28 and comprising a disclosed transgene.

Disclosed herein is a viral vector comprising the sequence set forth in SEQ ID NO:29 and comprising a disclosed transgene. Disclosed herein is a viral vector comprising the sequence set forth in SEQ ID NO:30 and comprising a disclosed transgene. Disclosed herein is a viral vector comprising the sequence set forth in SEQ ID NO:31 and comprising a disclosed transgene.

Disclosed herein is a viral vector comprising the sequence set forth in any one of SEQ ID NO:26-SEQ ID NO:31, and one or more of the following transgenes: APOE, APP, ATXN2, CHMP2B, DCTN1, FIG4, FUS, GBA, GRN, HNRNPA1, HTT, LRRK2, MATR3, OPTN, PARK7, PFN1, PRPH, PSEN1, SETX, SIGMAR1, SNCA, SOD1, SPG11, SQSTM1, TARDBP, TBK1, TBP, TRPM7, TUBA4A, UBQLN2, UCHL1, VAPB, VCP, VPS35, a fragment thereof, a variant thereof, or a chimera thereof.

Disclosed herein is a viral vector comprising the sequence set forth in SEQ ID NO:01, and one or more of the following transgenes: APOE, APP, ATXN2, CHMP2B, DCTN1, FIG4, FUS, GBA, GRN, HNRNPA1, HTT, LRRK2, MATR3, OPTN, PARK7, PFN1, PRPH, PSEN1, SETX, SIGMAR1, SNCA, SOD1, SPG11, SQSTM1, TARDBP, TBK1, TBP, TRPM7, TUBA4A, UBQLN2, UCHL1, VAPB, VCP, VPS35, a fragment thereof, a variant thereof, or a chimera thereof.

Disclosed herein is a virus producer cell line transduced with a disclosed viral vector. Disclosed herein is a virus producer cell line comprising a HDAC−/− genotype. Disclosed herein is a virus producer cell line comprising a HDAC1−/− genotype. Disclosed herein is a virus producer cell line comprising a HDAC2−/− genotype. Disclosed herein is a virus producer cell line comprising a HDAC3−/− genotype. Disclosed herein is a virus producer cell line comprising a HDAC4−/− genotype. Disclosed herein is a virus producer cell line comprising a HDAC6−/− genotype. Disclosed herein is a virus producer cell line comprising a HDAC8−/− genotype.

Disclosed herein is a kit comprising one or more disclosed compositions. Disclosed herein is a kit comprising a disclosed composition for a use in a disclosed method.

Disclosed herein is a method of developing HDAC depleted cells, the method comprising contacting one or more cells with a disclosed viral vector; wherein following transduction of the one or more cells, the expression and/or activity of the targeted HDAC is decreased and/or depleted.

Disclosed herein is a method of developing HDAC depleted cells, the method comprising contacting one or more cells with a viral vector comprising an isolated nucleic acid molecule comprising a nucleic acid sequence encoding (i) a Cas endonuclease, (ii) at least one guide RNA, and (iii) at least one transgene; wherein following transduction of the one or more cells, the expression and/or activity of the targeted HDAC is decreased and/or depleted.

Disclosed herein is a method of developing HDAC depleted cells, the method comprising contacting one or more cells with a viral vector comprising an isolated nucleic acid molecule comprising a nucleic acid sequence encoding (i) a fusion product, and (ii) at least one guide RNA; wherein following transduction of the one or more cells, the expression and/or activity of the targeted HDAC is decreased and/or depleted.

Disclosed herein is a method of increasing the packaging capacity of a viral vector, the method comprising contacting one or more cells with a disclosed viral vector; wherein following transduction of the one or more cells, the expression and/or activity of the targeted HDAC is decreased and/or depleted; and wherein the generated optimized viral vector has an increased packaging capacity. Disclosed herein is a method of increasing the packaging capacity of a viral vector, the method comprising contacting one or more cells with a viral vector comprising an isolated nucleic acid molecule comprising a nucleic acid sequence encoding (i) a Cas endonuclease, (ii) at least one guide RNA, and (iii) at least one transgene; wherein following transduction of the one or more cells, the expression and/or activity of the targeted HDAC is decreased and/or depleted; and wherein the generated optimized viral vector has an increased packaging capacity. Disclosed herein is a method of increasing the packaging capacity of a viral vector, the method comprising contacting one or more cells with a viral vector comprising an isolated nucleic acid molecule comprising a nucleic acid sequence encoding (i) a fusion product, and (ii) at least one guide RNA; wherein following transduction of the one or more cells, the expression and/or activity of the targeted HDAC is decreased and/or depleted; and wherein the generated optimized viral vector has an increased packaging capacity.

Disclosed herein is a method of increasing the titer of a viral vector, the method comprising contacting one or more cells with a disclosed viral vector; wherein following transduction of the one or more cells, the expression and/or activity of the targeted HDAC is decreased and/or depleted; and wherein the titer of the generated optimized viral vector is increased. Disclosed herein is a method of increasing the titer of a viral vector, the method comprising contacting one or more cells with a viral vector comprising an isolated nucleic acid molecule comprising a nucleic acid sequence encoding (i) a Cas endonuclease, (ii) at least one guide RNA, and (iii) at least one transgene; wherein following transduction of the one or more cells, the expression and/or activity of the targeted HDAC is decreased and/or depleted; and wherein the titer of the generated optimized viral vector is increased. Disclosed herein is a method of increasing the titer of a viral vector, the method comprising contacting one or more cells with a viral vector comprising an isolated nucleic acid molecule comprising a nucleic acid sequence encoding (i) a fusion product, and (ii) at least one guide RNA; wherein following transduction of the one or more cells, the expression and/or activity of the targeted HDAC is decreased and/or depleted; and wherein the titer of the generated optimized viral vector is increased.

Disclosed herein is a method of increasing the expression capacity of a viral vector, the method comprising contacting one or more cells with a disclosed viral vector; wherein following transduction of the one or more cells, the expression and/or activity of the targeted HDAC is decreased and/or depleted; and wherein the generated optimized viral vector has an increased expression capacity is increased. Disclosed herein is a method of increasing the expression capacity of a viral vector, the method comprising contacting one or more cells with a viral vector comprising an isolated nucleic acid molecule comprising a nucleic acid sequence encoding (i) a Cas endonuclease, (ii) at least one guide RNA, and (iii) at least one transgene; wherein following transduction of the one or more cells, the expression and/or activity of the targeted HDAC is decreased and/or depleted; and wherein the generated optimized viral vector has an increased expression capacity is increased. Disclosed herein is a method of increasing the expression capacity of a viral vector, the method comprising contacting one or more cells with a viral vector comprising an isolated nucleic acid molecule comprising a nucleic acid sequence encoding (i) a fusion product, and (ii) at least one guide RNA; wherein following transduction of the one or more cells, the expression and/or activity of the targeted HDAC is decreased and/or depleted; and wherein the generated optimized viral vector has an increased expression capacity is increased.

Disclosed herein is a method of decreasing the immunogenicity and/or toxicity of a viral vector, the method comprising contacting one or more cells with a disclosed viral vector; wherein following transduction of the one or more cells, the expression and/or activity of the targeted HDAC is decreased and/or depleted; and wherein the generated optimized viral vectors have decreased immunogenicity and/or toxicity. Disclosed herein is a method of decreasing the immunogenicity and/or toxicity of a viral vector, the method comprising contacting one or more cells with a viral vector comprising an isolated nucleic acid molecule comprising a nucleic acid sequence encoding (i) a Cas endonuclease, (ii) at least one guide RNA, and (iii) at least one transgene; wherein following transduction of the one or more cells, the expression and/or activity of the targeted HDAC is decreased and/or depleted; and wherein the generated optimized viral vectors have decreased immunogenicity and/or toxicity. Disclosed herein is a method of decreasing the immunogenicity and/or toxicity of a viral vector, the method comprising contacting one or more cells with a viral vector comprising an isolated nucleic acid molecule comprising a nucleic acid sequence encoding (i) a fusion product, and (ii) at least one guide RNA; wherein following transduction of the one or more cells, the expression and/or activity of the targeted HDAC is decreased and/or depleted; and wherein the generated optimized viral vectors have decreased immunogenicity and/or toxicity.

Disclosed herein is a method of increasing the ability of a viral vector to efficiently transduce one or more cells, the method comprising contacting one or more cells with a disclosed viral vector; wherein following transduction of the one or more cells, the expression and/or activity of the targeted HDAC is decreased and/or depleted; and wherein the generated optimized viral vectors have an increased ability to efficiently transduce cells. Disclosed herein is a method of increasing the ability of a viral vector to efficiently transduce one or more cells, the method comprising contacting one or more cells with a viral vector comprising an isolated nucleic acid molecule comprising a nucleic acid sequence encoding (i) a Cas endonuclease, (ii) at least one guide RNA, and (iii) at least one transgene; wherein following transduction of the one or more cells, the expression and/or activity of the targeted HDAC is decreased and/or depleted; and wherein the generated optimized viral vectors have an increased ability to efficiently transduce cells. Disclosed herein is a method of increasing the ability of a viral vector to efficiently transduce one or more cells, the method comprising contacting one or more cells with a viral vector comprising an isolated nucleic acid molecule comprising a nucleic acid sequence encoding (i) a fusion product, and (ii) at least one guide RNA; wherein following transduction of the one or more cells, the expression and/or activity of the targeted HDAC is decreased and/or depleted; and wherein the generated optimized viral vectors have an increased ability to efficiently transduce cells.

### VI. DETAILED DESCRIPTION

The present disclosure describes formulations, compounded compositions, kits, capsules, containers, and/or methods thereof. It is to be understood that the inventive aspects of which are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

### A. Relevant Definitions

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

This disclosure describes inventive concepts with reference to specific examples. However, the intent is to cover all modifications, equivalents, and alternatives of the inventive concepts that are consistent with this disclosure.

As used in the specification and the appended claims, the singular forms “a”, “an”, and “the” include plural referents unless the context clearly dictates otherwise.

The phrase “consisting essentially of” limits the scope of a claim to the recited components in a composition or the recited steps in a method as well as those that do not materially affect the basic and novel characteristic or characteristics of the claimed composition or claimed method.

The phrase “consisting of” excludes any component, step, or element that is not recited in the claim. The phrase “comprising” is synonymous with “including”, “containing”, or “characterized by”, and is inclusive or open-ended. “Comprising” does not exclude additional, unrecited components or steps.

As used herein, when referring to any numerical value, the term “at least” means a value falling within a range that is +10% of the stated value.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and 5 parts by weight component Y, X and Y are present at a weight ratio of 2:5, and are present in such ratio regardless of whether additional components are contained in the compound.

As used herein, the terms “optional” or “optionally” means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step.

As used herein, “CRISPR or clustered regularly interspaced short palindromic repeat” is an ideal tool for correction of genetic abnormalities associated with diseases such as Alzheimer's disease or LOAD. The system can be designed to target genomic DNA directly. A CRISPR system involves two main components: a Cas9 enzyme and a guide (gRNA). The gRNA contains a targeting sequence for DNA binding (at, for example, an NDD-related gene) and a scaffold sequence for Cas9 binding. Cas9 nuclease is often used to “knockout” target genes such as for example, an NDD-related gene. Also, multiple gRNAs can be employed to suppress or activate multiple genes simultaneously, hence increasing the treatment efficacy and reducing resistance potentially caused by new mutations in the target genes.

As used herein, “CRISPR-based endonucleases” include RNA-guided endonucleases that comprise at least one nuclease domain and at least one domain that interacts with a guide RNA. As known to the art, a guide RNA directs the CRISPR-based endonucleases to a targeted site in a nucleic acid at which site the CRISPR-based endonucleases cleaves at least one strand of the targeted nucleic acid sequence. As the guide RNA provides the specificity for the targeted cleavage, the CRISPR-based endonuclease is universal and can be used with different guide RNAs to cleave different target nucleic acid sequences. CRISPR-based endonucleases are RNA-guided endonucleases derived from CRISPR/Cas systems.

In an aspect, a disclosed CRISPR-based endonuclease can be derived from a CRISPR/Cas type I, type II, or type III system. Non-limiting examples of suitable CRISPR/Cas proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966.

In an aspect, a disclosed CRISPR-based endonuclease can be derived from a type II CRISPR/Cas system. For example, in an aspect, a CRISPR-based endonuclease can be derived from a Cas9 protein. The Cas9 protein can be from Streptococcus pyogenes, Streptococcus thermophilus, Streptococcus sp, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonfex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina. In an aspect, the CRISPR-based nuclease can be derived from a Cas9 protein from Streptococcus pyogenes.

As used herein, “dCas9” refers to enzymatically inactive form of Cas9, which can bind, but cannot cleave, DNA.

As used herein, “Protospacer Adjacent Motif” or “PAM” refers to a sequence adjacent to the target sequence that is necessary for Cas enzymes to bind target DNA.

A “protospacer sequence” refers to the target double stranded DNA and specifically to the portion of the target DNA (e.g., or target region in the genome) that is fully or substantially complementary (and hybridizes) to the spacer sequence of the CRISPR arrays. The protospacer sequence in a Type I system is directly flanked at the 3′ end by a PAM. A spacer is designed to be complementary to the protospacer.

In general, a gRNA (also referred to herein as “gRNA scaffold” interchangeably) can complex with a compatible nucleic acid-guided nuclease and can hybridize with a target sequence, thereby directing the nuclease to the target sequence. A subject nucleic acid-guided nuclease capable of complexing with a guide polynucleotide can be referred to as a nucleic acid-guided nuclease that is compatible with the gRNA. In addition, a gRNA capable of complexing with a nucleic acid-guided nuclease can be referred to as a guide polynucleotide or a guide nucleic acid that is compatible with the nucleic acid-guided nucleases.

A gRNA can include a scaffold sequence. In general, a “scaffold sequence” can include any sequence that has sufficient sequence to promote formation of a targetable nuclease complex, wherein the targetable nuclease complex includes, but is not limited to, a nucleic acid-guided nuclease and a guide polynucleotide can include a scaffold sequence and a guide sequence.

Sufficient sequence within the scaffold sequence to promote formation of a targetable nuclease complex can include a degree of complementarity along the length of two sequence regions within the scaffold sequence, such as one or two sequence regions involved in forming a secondary structure. In an aspect, the one or two sequence regions are included or encoded on the same polynucleotide. In an aspect, the one or two sequence regions are included or encoded on separate polynucleotides. Optimal alignment can be determined by any suitable alignment algorithm, and can further account for secondary structures, such as self-complementarity within either the one or two sequence regions. In an aspect, the degree of complementarity between the one or two sequence regions along the length of the shorter of the two when optimally aligned can be about or more than about 25%, 30%, 40%, 50%, 60%, 70%, 80%, 90%, 95%, 97.5%, 99%, or higher. In an aspect, at least one of the two sequence regions can be about or more than about 5, 6, 7, 8, 9, 10, 11, 12, 13, 14, 15, 16, 17, 18, 19, 20, 25, 30, 40, 50, or more nucleotides in length.

A scaffold sequence of a subject guide polynucleotide can comprise a secondary structure. A secondary structure can comprise a pseudoknot region. In an aspect, binding kinetics of a guide polynucleotide to a nucleic acid-guided nuclease is determined in part by secondary structures within the scaffold sequence. In an aspect, binding kinetics of a guide polynucleotide to a nucleic acid-guided nuclease is determined in part by nucleic acid sequence with the scaffold sequence.

“Promoter” as used herein means a synthetic or naturally-derived molecule which is capable of conferring, activating, or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter can be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the EFS promoter, bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter, human U6 (hU6) promoter, and CMV IE promoter.

The terms “transformation,” “transfection,” and “transduction” as used interchangeably herein refer to the introduction of a heterologous nucleic acid molecule into a cell. Such introduction into a cell can be stable or transient. Thus, in an aspect, a host cell or host organism is stably transformed with a polynucleotide of the disclosure. In an aspect, a host cell or host organism is transiently transformed with a polynucleotide of the disclosure. “Transient transformation” in the context of a polynucleotide means that a polynucleotide is introduced into the cell and does not integrate into the genome of the cell. By “stably introducing” or “stably introduced” in the context of a polynucleotide introduced into a cell is intended that the introduced polynucleotide is stably incorporated into the genome of the cell, and thus the cell is stably transformed with the polynucleotide. “Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. “Genome” as used herein also includes the nuclear, the plasmid and the plastid genome, and therefore includes integration of the nucleic acid construct into, for example, the chloroplast or mitochondrial genome. Stable transformation as used herein can also refer to a transgene that is maintained extrachromasomally, for example, as a minichromosome or a plasmid. In an aspect, the nucleotide sequences, constructs, expression cassettes can be expressed transiently and/or they can be stably incorporated into the genome of the host organism.

“Transgene” as used herein refers to a gene or genetic material containing a gene sequence that has been isolated from one organism and is introduced into a different organism. This non-native segment of DNA may retain the ability to produce RNA or protein in the transgenic organism, or it may alter the normal function of the transgenic organism's genetic code. The introduction of a transgene has the potential to change the phenotype of an organism.

As used herein, the term “subject” refers to the target of administration, e.g., a human being. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, or rodent. The term does not denote a particular age or sex, and thus, adult and child subjects, as well as fetuses, whether male or female, are intended to be covered. In an aspect, a subject can be a human patient. In an aspect, a subject can have a genetic disease or disorder or a neurological disease or disorder, be suspected of having a genetic disease or disorder or a neurological disease or disorder, or be at risk of developing a genetic disease or disorder or a neurological disease or disorder.

“Target gene” as used herein refers to any nucleotide sequence encoding a known or putative gene product. The target gene can be a mutated gene involved in a genetic disease or disorder or a neurological disease or disorder.

“Target region” as used herein refers to the region of the target gene and/or chromosome to which the composition for epigenome modification of the target gene is designed to bind and modify.

As used herein, the term “diagnosed” means having been subjected to an examination by a person of skill, for example, a physician, and found to have a condition that can be diagnosed or treated by one or more of the disclosed isolated nucleic acid molecule, a disclosed shRNA targeting an HDAC, a disclosed viral vector, a disclosed producer cell, or a combination thereof, or by one or more of the disclosed methods. For example, “diagnosed with a genetic disease or disorder or a neurological disease or disorder” means having been subjected to an examination by a person of skill, for example, a physician, and found to have a condition that can be treated by one or more a disclosed isolated nucleic acid molecule, a disclosed shRNA targeting an HDAC, a disclosed viral vector, a disclosed producer cell, or a combination thereof, or by one or more of the disclosed methods. For example, “suspected of having a genetic disease or disorder or a neurological disease or disorder” can mean having been subjected to an examination by a person of skill, for example, a physician, and found to have a condition that can likely be treated by one or more of a disclosed isolated nucleic acid molecule, a disclosed shRNA targeting an HDAC, a disclosed viral vector, a disclosed producer cell, or a combination thereof, or by one or more of the disclosed methods. In an aspect, an examination can be physical, can involve various tests (e.g., blood tests, genotyping, biopsies, etc.) and assays (e.g., enzymatic assay), or a combination thereof.

A “patient” can refer to a subject that has been diagnosed with or is suspected of having a genetic disease or disorder or a neurological disease or disorder. In an aspect, a patient can refer to a subject that has been diagnosed with or is suspected of having a genetic disease or disorder, and is seeking treatment or receiving treatment for a genetic disease or disorder.

As used herein, the phrase “identified to be in need of treatment for a disorder,” or the like, refers to selection of a subject based upon need for treatment of the disorder. For example, a subject can be identified as having a need for treatment of a genetic disease or disorder or a neurological disease or disorder based upon an earlier diagnosis by a person of skill and thereafter subjected to treatment for the genetic disease or disorder or a neurological disease or disorder. In an aspect, the identification can be performed by a person different from the person making the diagnosis. In an aspect, the administration can be performed by one who performed the diagnosis.

As used herein, the terms “neurological diseases” or “neurological disorders” are used interchangeably and refer to a host of undesirable conditions affecting neurons in the brain of a subject. These diseases include but are not limited to the following: Alzheimer's disease, Parkinson's disease, Huntington's disease, Pick's disease, Kufs disease, Lewy body disease, neurofibrillary tangles, Rosenthal fibers, Mallory's hyaline, senile dementia, myasthenia gravis, Gilles de la Tourette's syndrome, multiple sclerosis (MS), amyotrophic lateral sclerosis (ALS), progressive supranuclear palsy (PSP), epilepsy, Creutzfeldt-Jakob disease, deafness-dytonia syndrome, Leigh syndrome, Leber hereditary optic neuropathy (LHON), parkinsonism, dystonia, motor neuron disease, neuropathy-ataxia and retinitis pimentosa (NARP), maternal inherited Leigh syndrome (MILS), Friedreich ataxia, hereditary spastic paraplegia, Mohr-Tranebjaerg syndrome, Wilson disease, sporatic Alzheimer's disease, sporadic amyotrophic lateral sclerosis, sporadic Parkinson's disease, autonomic function disorders, hypertension, sleep disorders, neuropsychiatric disorders, depression, schizophrenia, schizoaffective disorder, korsakoff's psychosis, mania, anxiety disorders, phobic disorder, learning or memory disorders, amnesia or age-related memory loss, attention deficit disorder, dysthymic disorder, major depressive disorder, obsessive-compulsive disorder, psychoactive substance use disorders, panic disorder, bipolar affective disorder, severe bipolar affective (mood) disorder (BP-1), migraines, hyperactivity and movement disorders.

As used herein, the term “agent which prevents or reduces symptoms of the neurological disorder” or “agent used for the treatment of a neurological disorder” refers to those drugs that are used for the treatment of one or more of the disclosed neurological diseases and disorders. Examples of such agents include, but are not limited to, the following: anticholinergics, such as trihexyphenidyl (Artane®), benztropine (Cogentin®), ethopropazine (Parsitan®); benzodiazepines, such as diazepam (Valium®), clonazepam (Klonopin®), lorazepam (Ativan®); baclofen (Lioresal®), dopaminergic agents such as levodopa (Sinemet®) and bromocriptine (Parlodel®); tetrabenazine (Xenazine®), dopamine-depleting agents, ritonavir, lopinavir, and the like. Other agents that treat, prevent, inhibit, and/or ameliorate symptoms and/or complications of a neurological disorder and/or a neurodegenerative disease include the following: Acamprosate tablets (Campral EC), Adrenaline (epinephrine) (Emerade, EpiPen, Jext), Agomelatine tablets (Valdoxan), Almotriptan (Almogran), Amantadine, Amisulpride (Solian), Amitriptyline (Elavil), Apomorphine (APO-go, Dacepton), Aripiprazole (Abilify), Aripiprazole long-acting injection (Abilify Maintena), Asenapine tablets (Sycrest), Atomoxetine (Strattera), Baclofen (Lyflex, Lioresal), Botulinum toxin type A (Botox), Bromocriptine (Parlodel), Buccal midazolam (Buccolam, Epistatus), Buprenorphine (BuTrans, Hapoctasin, Temgesic, Tephine, Transtec), Buspirone, Cabergoline tablets (Dostinex, Cabaser), Carbamazepine (Curatil, Tegretol), Chlordiazepoxide (Librium), Chlorpromazine, Citalopram (Cipramil, Celexa), Clobazam (Frisium, Perizam, Tapclob, Zacco), Clomethiazole, Clomipramine, Clonazepam, Clozapine (Clozaril, Denzapine, Zaponex), Co-beneldopa (Madopar), Co-careldopa (Sinemet), Dantrolene (Dantrium), Dexamfetamine (Amfexa), Diazepam (Diazemuls, Stesolid), Divalproex sodium (Depakote), Donepezil (Aricept), Doxepin capsules, Duloxetine (Cymbalta, Depalta, Duciltia), Eletriptan (Relpax), Entacapone (Comtess), Escitalopram (Cipralex), Eslicarbazepine (Zebinix), Ethosuximide, Fingolimod capsules (Gilenya), Fluoxetine (Olena, Prozac, Prozep), Flupentixol long-acting injection (Depixol, Psytixol), Flupentixol tablets (Depixol, Fluanxol), Fluphenazine long-acting injection (Modecate), Fluvoxamine tablets (Faverin), Frovatriptan for migraine (Migard), Gabapentin (Neurontin), Galantamine (Acumor, Consion, Elmino, Gaalin, Galsya, Galzemic, Gatalin, Gazylan, Lotprosin, Luventa, Reminyl), Haloperidol (Haldol, Serenace), Haloperidol long-acting injection (Haldol Decanoate), Hydromorphone (Palladone), Imipramine tablets and liquid medicine, Lacosamide (Vimpat), Lamotrigine (Lamictal), Levetiracetam for epilepsy (Keppra, Desitrend), Levomepromazine tablets (Nozinan), Lisdexamfetamine (Elvanse), Lithium tablets and liquid medicine (Camcolit, Liskonum, Priadel, Li-Liquid), Lofepramine, Loprazolam, Lorazepam, Lormetazepam tablets, Lurasidone (Latuda), Melatonin tablets (Circadin, Slenyto), Memantine (Ebixa, Nemtadine), Methylphenidate (Concerta, Equasym, Medikinet, Ritalin, Tranquilyn), Mianserin, Midodrine (Bramox), Mirtazapine (Zispin SolTab), Moclobemide (Manerix), Modafinil tablets (Provigil), Morphine (Morphgesic, Oramorph, Zomorph), Naratriptan (Naramig), Neostigmine, Nitrazepam (Mogadon), Nortriptyline tablets, Olanzapine (Zalasta, Zyprexa), Olanzapine long-acting injection (Zypadhera), Orlistat capsules (Alli, Beacita, Orlos, Xenical), Orphenadrine, Oxazepam, Oxcarbazepine (Trileptal), Oxycodone (Abtard, Longtec, OxyContin, OxyNorm, Shortec), Paliperidone (Invega), Paliperidone long-acting injection (Xeplion, Trevicta), Paroxetine (Seroxat), Perampanel (Fycompa), Pergolide, Pericyazine, Phenobarbital, Phenytoin (Epanutin), Piracetam (Nootropil), Pizotifen tablets, Pramipexole tablets (Mirapexin, Oprymea, Pipexus, Glepark), Pregabalin (Alzain, Axalid, Lecaent, Lyrica), Primidone, Prochlorperazine (Buccastem, Stemetil), Procyclidine (Kemadrin), Pyridostigmine (Mestinon), Quetiapine (Seroquel), Rasagiline (Azilect), Reboxetine tablets (Edronax), Risperidone (Risperdal), Risperidone long-acting injection (Risperdal Consta), Rivastigmine (Alzest, Exelon, Nimvastid), Rizatriptan for migraine (Maxalt), Ropinirole tablets (Requip, Adartrel), Rotigotine patches (Neupro), Rufinamide for epilepsy (Inovelon), Selegiline (Eldepryl), Sertraline (Lustral, Zoloft), Sodium oxybate (Xyrem), Sodium valproate (Epilim, Episenta, Epival, Convulex), Sulpiride, Sumatriptan (Imigran), Temazepam, Tetrabenazine tablets (Tardiben, Xenazine), Tiagabine (Gabitril), Tizanidine, Tolcapone (Tasmar), Topiramate (Topamax), Topiramate (Topamax), Trazodone (Molipaxin), Trihexyphenidyl, Trimipramine, Valproate semisodium (Belvo, Depakote, Syonell), Venlafaxine (Efexor XL, Effexor XR), Vigabatrin (Sabril, Kigabeq), Vortioxetine (Brintellix), Zolmitriptan (Zomig), Zolpidem tablets (Stilnoct), Zonisamide (Zonegran, Desizon), Zopiclone tablets (Zimovane), and Zuclopenthixol (Clopixol). In an aspect, any one or combination of these agents can be a therapeutic agent used in a disclosed method.

“Identical” or “identity” as used herein in the context of two or more nucleic acids or polypeptide sequences means that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity.

In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree, and distribution of charged regions) is recognized in the art as typically involving a minor change. These minor changes can be identified, in part, by considering the hydropathic index of amino acids, as understood in the art. The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

“Vector” as used herein means a nucleic acid sequence containing an origin of replication. A vector can be a viral vector, bacteriophage, bacterial artificial chromosome or yeast artificial chromosome. A vector can be a DNA or RNA vector. A vector can be a self-replicating extrachromosomal vector, and preferably, is a DNA plasmid.

As used herein, “inhibit,” “inhibiting”, and “inhibition” mean to diminish or decrease an activity, expression, level, response, condition, severity, disease, or other biological parameter. This can include, but is not limited to, the complete ablation of the activity, expression, level, response, condition, severity, disease, or other biological parameter. This can also include, for example, a 10% inhibition or reduction in the activity, expression, level, response, condition, severity, disease, or other biological parameter as compared to the native or control level (e.g., a wild-type or normal HDAC genotype). Thus, in an aspect, the inhibition or reduction can be a 10%, 20%, 30%, 40%, 50%, 60%, 70%, 80%, 90%, 100%, or any amount of reduction in between as compared to native or control levels. In an aspect, the inhibition or reduction can be 10-20%, 20-30%, 30-40%, 40-50%, 50-60%, 60-70%, 70-80%, 80-90%, or 90-100% as compared to native or control levels. In an aspect, the inhibition or reduction can be 0-25%, 25-50%, 50-75%, or 75-100% as compared to native or control levels. In an aspect, a native or control level can be a pre-disease or pre-disorder level.

As used herein, the terms “increase,” “increasing,” “increased,” “enhance,” “enhanced,” “enhancing,” and “enhancement” (and grammatical variations thereof) describe an elevation of at least about 25%, 50%, 75%, 100%, 150%, 200%, 300%, 400%, 500% or more as compared to a control (e.g., increased titer, increased expression capacity, increased packaging capacity, increased transduction efficiency, or any combination thereof).

An “isolated” polynucleotide or an “isolated” polypeptide is a nucleotide sequence or polypeptide sequence that, by the hand of man, exists apart from its native environment and is therefore not a product of nature. In an aspect, the polynucleotides and polypeptides of the disclosure are “isolated.” An isolated polynucleotide or polypeptide can exist in a purified form that is at least partially separated from at least some of the other components of the naturally occurring organism or virus, for example, the cell or viral structural components or other polypeptides or polynucleotides commonly found associated with the polypeptide or polynucleotide. In an aspect, the isolated polynucleotide and/or the isolated polypeptide is at least about 1%, 5%, 10%, 20%, 30%, 40%, 50%, 60%, 70%, 80%, 90%, 95%, or more pure.

“Multicistronic” or “polycistronic” as used interchangeable herein refers to a polynucleotide possessing more than one coding region to produce more than one protein from the same polynucleotide. The polycistronic polynucleotide sequence can include (internal ribosome-entry site (IRES), cleavage peptides (p2A, t2A and others), utilization of different promoters, etc.

As used herein, the term “percent sequence identity” or “percent identity” refers to the percentage of identical nucleotides in a linear polynucleotide of a reference (“query”) polynucleotide molecule (or its complementary strand) as compared to a test (“subject”) polynucleotide molecule (or its complementary strand) when the two sequences are optimally aligned. In an aspect, “percent identity” can refer to the percentage of identical amino acids in an amino acid sequence.

“Mutant gene” or “mutated gene” as used interchangeably herein refers to a gene that has undergone a detectable mutation. A mutant gene has undergone a change, such as the loss, gain, or exchange of genetic material, which affects the normal transmission and expression of the gene.

A “native” or “wild type” nucleic acid, nucleotide sequence, polypeptide or amino acid sequence refers to a naturally occurring or endogenous nucleic acid, nucleotide sequence, polypeptide or amino acid sequence. Thus, for example, a “wild type mRNA” is an mRNA that is naturally occurring in or endogenous to the organism. A “homologous” nucleic acid is a nucleotide sequence naturally associated with a host cell into which it is introduced.

The words “treat” or “treating” or “treatment” include palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder (such as a genetic disease or disorder or a neurological disease or disorder). In an aspect, the terms cover any treatment of a subject, including a mammal (e.g., a human), and includes: (i) preventing the undesired physiological change, disease, pathological condition, or disorder from occurring in a subject that can be predisposed to the disease but has not yet been diagnosed as having it; (ii) inhibiting the physiological change, disease, pathological condition, or disorder, i.e., arresting its development; or (iii) relieving the physiological change, disease, pathological condition, or disorder, i.e., causing regression of the disease. For example, in an aspect, treating a genetic disease or disorder or a neurological disease or disorder can reduce the severity of an established a genetic disease or disorder or a neurological disease or disorder in a subject by 1%-100% as compared to a control (such as, for example, an individual not having a genetic disease or disorder or a neurological disease or disorder). In an aspect, treating can refer to a 1%, 2%, 3%, 4%, 5%, 6%, 7%, 8%, 9%, 10%, 20%, 30%, 40%, 50%, 60%, 70%, 80%, 90%, or 100% reduction in the severity of a genetic disease or disorder or a neurological disease or disorder. For example, treating a genetic disease or disorder or a neurological disease or disorder can reduce one or more symptoms in a subject by 1%-100% as compared to a control (such as, for example, an individual not having a genetic disease or disorder or a neurological disease or disorder). In an aspect, treating can refer to 1%, 2%, 3% 4%, 5%, 6%, 7% 8%, 9% 10%, 20%, 30%, 40%, 50%, 60%, 70%, 80%, 90%, 100% reduction of one or more symptoms of an established genetic disease or disorder or a neurological disease or disorder. It is understood that treatment does not necessarily refer to a cure or complete ablation or eradication of a genetic disease or disorder or a neurological disease or disorder. However, in an aspect, treatment can refer to a cure or complete ablation or eradication of a genetic disease or disorder or a neurological disease or disorder.

As used herein, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, preventing a genetic disease or disorder or a neurological disease or disorder is intended. The words “prevent” and “preventing” and “prevention” also refer to prophylactic or preventative measures for protecting or precluding a subject (e.g., an individual) not having a given genetic disease or disorder or a neurological disease or disorder related complication from progressing to that complication.

As used herein, “short hairpin RNA” or “shRNA” refers to sequence usually encoded in a DNA vector that can be introduced into cells via plasmid transfection or viral transduction. As known to the art, shRNA molecules can be divided into two main categories based on their designs: simple stem-loop and microRNA-adapted shRNA. A simple stem-loop shRNA is often transcribed under the control of an RNA Polymerase III (Pol III) promoter. The 50-70 nucleotide transcript forms a stem-loop structure consisting of a 19 to 29 bp region of double-strand RNA (the stem) bridged by a region of predominantly single-strand RNA (the loop) and a dinucleotide 3′ overhang. The simple stem-loop shRNA is transcribed in the nucleus and enters the RNAi pathway similar to a pre-microRNA. The longer (>250 nucleotide) microRNA-adapted shRNA is a design that more closely resembles native pri-microRNA molecules and consists of a shRNA stem structure which may include microRNA-like mismatches, bridged by a loop and flanked by 5′ and 3′ endogenous microRNA sequences. The microRNA-adapted shRNA, like the simple stem-loop hairpin, is also transcribed in the nucleus but is thought to enter the RNAi pathway earlier similar to an endogenous pri-microRNA.

As used herein, “operably linked” means that expression of a gene is under the control of a promoter with which it is spatially connected. A promoter can be positioned 5′ (upstream) or 3′ (downstream) of a gene under its control. The distance between the promoter and a gene can be approximately the same as the distance between that promoter and the gene it controls in the gene from which the promoter is derived. As is known in the art, variation in this distance can be accommodated without loss of promoter function.

“Adeno-associated virus” or “AAV” as used interchangeably herein can refer to a small virus belonging to the genus Dependovirus of the Parvoviridae family that infects humans and some other primate species. AAV is not currently known to cause disease and consequently the virus causes a very mild immune response.

The term “lentiviral vector” as used herein can refer to a vector including one or more “heterologous” (i.e., non-lentiviral) nucleic acid sequences. In an aspect, lentiviral vectors herein may contain non-coding sequences of one or more proteins from a lentivirus. A “lentiviral transfer vector” for use herein may include a heterologous nucleic acid sequence, for example, to be transferred into a cell, and may further include, for example, one or more lentiviral genes, or portions thereof. A “lentiviral packaging vector” for use herein may include one or more genes encoding lentiviral proteins, or portions thereof. For example, a lentiviral envelope protein may include a gene encoding an envelope (Env) protein, or a portion thereof. In an aspect, host cells can be transfected with lentiviral vectors, and optionally additional vectors for expressing lentiviral packaging proteins (e.g., VSV-G, Rev, and Gag/Pol) to produce lentiviral particles in the culture medium.

As used herein, “chimeric” can refer to a nucleic acid molecule and/or a polypeptide in which at least two components are derived from different sources (e.g., different organisms, different coding regions). Also as used herein, chimeric refers to a construct comprising a polypeptide linked to a nucleic acid.

Epigenome modification” as used herein refers to a modification or change in one or more chromosomes that affect gene activity and expression that does not derive from a modification of the genome. An epigenome modification relates to a functionally relevant change to the genome that does not involve a change in the nucleotide sequence. Epigenome modifications may include a modification to a histone, such as acetylation, methylation, phosphorylation, ubiquitination, and/or sumoylation. Epigenome modifications may include a modification to DNA, such as methylation.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The coding sequence can be codon optimized.

“Fusion protein” as used herein refers to a chimeric protein created through the joining of two or more genes that originally coded for separate proteins (e.g., Cas and a polypeptide having enzymatic activity). The translation of the fusion gene results in a single polypeptide with functional properties derived from each of the original proteins.

As used herein, the terms “administering” and “administration” refer to any method of providing one or more of the disclosed therapeutic agents, disclosed pharmaceutical formulations, or a combination thereof to a subject. Such methods are well known to those skilled in the art and include, but are not limited to, the following routes: oral administration, transdermal administration, administration by inhalation, nasal administration, topical administration, in utero administration, intrahepatic administration, intravaginal administration, ophthalmic administration, intraaural administration, otic administration, intracerebral administration, rectal administration, sublingual administration, buccal administration, and parenteral administration, including injectable such as intravenous administration, intra-CSF administration, intra-arterial administration, intramuscular administration, and subcutaneous administration. Administration can also include hepatic intra-arterial administration or administration through the hepatic portal vein (HPV). Administration of a disclosed therapeutic agent, a disclosed pharmaceutical composition, or a combination thereof can comprise administration directly into the CNS (e.g., intraparenchymal, intracerebroventriular, inthrathecal cisternal, intrathecal (lumbar), deep gray matter delivery, convection-enhanced delivery to deep gray matter) or the PNS. Administration can be continuous or intermittent. Administration can comprise administering a viral vector and/or generated optimized viral vector.

In an aspect, a “therapeutic agent” can be a “biologically active agent” or “biologic active agent” or “bioactive agent”, which refers to an agent that is capable of providing a local or systemic biological, physiological, or therapeutic effect in the biological system to which it is applied. For example, the bioactive agent can act to control infection or inflammation, enhance cell growth and tissue regeneration, control tumor growth, act as an analgesic, promote anti-cell attachment, and enhance bone growth, among other functions. Other suitable bioactive agents can include anti-viral agents, vaccines, hormones, antibodies (including active antibody fragments sFv, Fv, and Fab fragments), aptamers, peptide mimetics, functional nucleic acids, therapeutic proteins, peptides, or nucleic acids. Other bioactive agents include prodrugs, which are agents that are not biologically active when administered but, upon administration to a subject are converted to bioactive agents through metabolism or some other mechanism. Additionally, any of the compositions of the invention can contain combinations of two or more bioactive agents. It is understood that a biologically active agent can be used in connection with administration to various subjects, for example, to humans (i.e., medical administration) or to animals (i.e., veterinary administration). As used herein, the recitation of a biologically active agent inherently encompasses the pharmaceutically acceptable salts thereof.

As used herein, the term “pharmaceutically active agent” includes a “drug” or a “vaccine” and means a molecule, group of molecules, complex or substance administered to an organism for diagnostic, therapeutic, preventative medical, or veterinary purposes. This term includes externally and internally administered topical, localized and systemic human and animal pharmaceuticals, treatments, remedies, nutraceuticals, cosmeceuticals, biologicals, devices, diagnostics and contraceptives, including preparations useful in clinical and veterinary screening, prevention, prophylaxis, healing, wellness, detection, imaging, diagnosis, therapy, surgery, monitoring, cosmetics, prosthetics, forensics and the like. This term may also be used in reference to agriceutical, workplace, military, industrial and environmental therapeutics or remedies comprising selected molecules or selected nucleic acid sequences capable of recognizing cellular receptors, membrane receptors, hormone receptors, therapeutic receptors, microbes, viruses or selected targets comprising or capable of contacting plants, animals and/or humans. This term can also specifically include nucleic acids and compounds comprising nucleic acids that produce a bioactive effect, for example deoxyribonucleic acid (DNA) or ribonucleic acid (RNA).

Pharmaceutically active agents include the herein disclosed categories and specific examples. It is not intended that the category be limited by the specific examples. Those of ordinary skill in the art will recognize also numerous other compounds that fall within the categories and that are useful according to the invention. Examples include a radiosensitizer, the combination of a radiosensitizer and a chemotherapeutic, a steroid, a xanthine, a beta-2-agonist bronchodilator, an anti-inflammatory agent, an analgesic agent, a calcium antagonist, an angiotensin-converting enzyme inhibitors, a beta-blocker, a centrally active alpha-agonist, an alpha-1-antagonist, carbonic anhydrase inhibitors, prostaglandin analogs, a combination of an alpha agonist and a beta blocker, a combination of a carbonic anhydrase inhibitor and a beta blocker, an anticholinergic/antispasmodic agent, a vasopressin analogue, an antiarrhythmic agent, an antiparkinsonian agent, an antiangina/antihypertensive agent, an anticoagulant agent, an antiplatelet agent, a sedative, an ansiolytic agent, a peptidic agent, a biopolymeric agent, an antineoplastic agent, a laxative, an antidiarrheal agent, an antimicrobial agent, an antifungal agent, or a vaccine. In a further aspect, the pharmaceutically active agent can be coumarin, albumin, bromolidine, steroids such as betamethasone, dexamethasone, methylprednisolone, prednisolone, prednisone, triamcinolone, budesonide, hydrocortisone, and pharmaceutically acceptable hydrocortisone derivatives; xanthines such as theophylline and doxophylline; beta-2-agonist bronchodilators such as salbutamol, fenterol, clenbuterol, bambuterol, salmeterol, fenoterol; antiinflammatory agents, including antiasthmatic anti-inflammatory agents, antiarthritis antiinflammatory agents, and non-steroidal antiinflammatory agents, examples of which include but are not limited to sulfides, mesalamine, budesonide, salazopyrin, diclofenac, pharmaceutically acceptable diclofenac salts, nimesulide, naproxene, acetominophen, ibuprofen, ketoprofen and piroxicam; analgesic agents such as salicylates; calcium channel blockers such as nifedipine, amlodipine, and nicardipine; angiotensin-converting enzyme inhibitors such as captopril, benazepril hydrochloride, fosinopril sodium, trandolapril, ramipril, lisinopril, enalapril, quinapril hydrochloride, and moexipril hydrochloride; beta-blockers (i.e., beta adrenergic blocking agents) such as sotalol hydrochloride, timolol maleate, timol hemihydrate, levobunolol hydrochloride, esmolol hydrochloride, carteolol, propanolol hydrochloride, betaxolol hydrochloride, penbutolol sulfate, metoprolol tartrate, metoprolol succinate, acebutolol hydrochloride, atenolol, pindolol, and bisoprolol fumarate; centrally active alpha-2-agonists (i.e., alpha adrenergic receptor agonist) such as clonidine, brimonidine tartrate, and apraclonidine hydrochloride; alpha-1-antagonists such as doxazosin and prazosin; anticholinergic/antispasmodic agents such as dicyclomine hydrochloride, scopolamine hydrobromide, glycopyrrolate, clidinium bromide, flavoxate, and oxybutynin; vasopressin analogues such as vasopressin and desmopressin; prostaglandin analogs such as latanoprost, travoprost, and bimatoprost; cholinergics (i.e., acetylcholine receptor agonists) such as pilocarpine hydrochloride and carbachol; glutamate receptor agonists such as the N-methyl D-aspartate receptor agonist memantine; anti-Vascular endothelial growth factor (VEGF) aptamers such as pegaptanib; anti-VEGF antibodies (including but not limited to anti-VEGF-A antibodies) such as ranibizumab and bevacizumab; carbonic anhydrase inhibitors such as methazolamide, brinzolamide, dorzolamide hydrochloride, and acetazolamide; antiarrhythmic agents such as quinidine, lidocaine, tocainide hydrochloride, mexiletine hydrochloride, digoxin, verapamil hydrochloride, propafenone hydrochloride, flecaimide acetate, procainamide hydrochloride, moricizine hydrochloride, and diisopyramide phosphate; antiparkinsonian agents, such as dopamine, L-Dopa/Carbidopa, selegiline, dihydroergocryptine, pergolide, lisuride, apomorphine, and bromocryptine; antiangina agents and antihypertensive agents such as isosorbide mononitrate, isosorbide dinitrate, propranolol, atenolol and verapamil; anticoagulant and antiplatelet agents such as coumadin, warfarin, acetylsalicylic acid, and ticlopidine; sedatives such as benzodiazapines and barbiturates; ansiolytic agents such as lorazepam, bromazepam, and diazepam; peptidic and biopolymeric agents such as calcitonin, leuprolide and other LHRH agonists, hirudin, cyclosporin, insulin, somatostatin, protirelin, interferon, desmopressin, somatotropin, thymopentin, pidotimod, erythropoietin, interleukins, melatonin, granulocyte/macrophage-CSF, and heparin; antineoplastic agents such as etoposide, etoposide phosphate, cyclophosphamide, methotrexate, 5-fluorouracil, vincristine, doxorubicin, cisplatin, hydroxyurea, leucovorin calcium, tamoxifen, flutamide, asparaginase, altretamine, mitotane, and procarbazine hydrochloride; laxatives such as senna concentrate, casanthranol, bisacodyl, and sodium picosulphate; antidiarrheal agents such as difenoxine hydrochloride, loperamide hydrochloride, furazolidone, diphenoxylate hydrochloride, and microorganisms; vaccines such as bacterial and viral vaccines; antimicrobial agents such as penicillins, cephalosporins, and macrolides, antifungal agents such as imidazolic and triazolic derivatives; and nucleic acids such as DNA sequences encoding for biological proteins, and antisense oligonucleotides. It is understood that a pharmaceutically active agent can be used in connection with administration to various subjects, for example, to humans (i.e., medical administration) or to animals (i.e., veterinary administration). As used herein, the recitation of a pharmaceutically active agent inherently encompasses the pharmaceutically acceptable salts thereof.

In an aspect, a “therapeutic agent” can be any agent that effects a desired clinical outcome in a subject having a genetic disease or disorder or a neurological disease or disorder, suspected of having a genetic disease or disorder or a neurological disease or disorder, and/or likely to develop or acquire a genetic disease or disorder or a neurological disease or disorder. In an aspect, a disclosed therapeutic agent can be an oligonucleotide therapeutic agent. A disclosed oligonucleotide therapeutic agent can comprise a single-stranded or double-stranded DNA, iRNA, shRNA, siRNA, mRNA, non-coding RNA (ncRNA), an antisense molecule, miRNA, a morpholino, a peptide-nucleic acid (PNA), or an analog or conjugate thereof. In an aspect, a disclosed oligonucleotide therapeutic agent can be an ASO or an RNAi. In an aspect, a disclosed oligonucleotide therapeutic agent can comprise one or more modifications at any position applicable.

In an aspect, the skilled person can determine an efficacious dose, an efficacious schedule, and an efficacious route of administration for one or more disclosed isolated nucleic acid molecules, disclosed shRNAs targeting an HDAC, disclosed viral vectors, disclosed producer cells, or a combination thereof, to treat or prevent a genetic disease or disorder or a neurological disease or disorder. In an aspect, the skilled person can also alter, change, or modify an aspect of an administering step to improve efficacy of one or more disclosed isolated nucleic acid molecules, disclosed shRNAs targeting an HDAC, disclosed viral vectors, disclosed producer cells, or a combination thereof. In an aspect, the skilled person can determine an efficacious dose, an efficacious schedule, and an efficacious route of administration for one or more disclosed isolated nucleic acid molecules, disclosed shRNAs targeting an HDAC, disclosed viral vectors, disclosed producer cells, or a combination thereof.

As used herein, “modifying the method” can comprise modifying or changing one or more features or aspects of one or more steps of a disclosed method. For example, in an aspect, disclosed a method can be altered by changing the amount of one or more disclosed isolated nucleic acid molecules, disclosed shRNAs targeting an HDAC, disclosed viral vectors, disclosed producer cells, or a combination thereof administered to a subject, or by changing the frequency of administration of one or more disclosed isolated nucleic acid molecules, disclosed shRNAs targeting an HDAC, disclosed viral vectors, disclosed producer cells, or a combination thereof, or by changing the duration of time one or more disclosed isolated nucleic acid molecules, disclosed shRNAs targeting an HDAC, disclosed viral vectors, disclosed producer cells, or a combination thereof are administered to a subject.

As used herein, “concurrently” means (1) simultaneously in time, or (2) at different times during the course of a common treatment schedule.

The term “contacting” as used herein refers to bringing one or more disclosed isolated nucleic acid molecules, disclosed shRNAs targeting an HDAC, disclosed viral vectors, disclosed producer cells, or a combination thereof together with a target area or intended target area in such a manner that the one or more disclosed isolated nucleic acid molecules, disclosed shRNAs targeting an HDAC, disclosed viral vectors, disclosed producer cells, or a combination thereof exert an effect on the intended target or targeted area either directly or indirectly. A target area can be a collection of cells or a cell line (such as a mammalian cell line). A target can comprise dividing cells and/or non-dividing cells. A target are can be cells to be transduced by a generated optimized viral vector such as, for example, mammalian cells (e.g., human, equine, bovine, porcine, canine, feline, or rodent cells). In an aspect, target cells to be transduced by a generated optimized viral vector can be in a subject. In an aspect, a subject can be a human subject. In an aspect, target cells to be transduced by a generated optimized viral vector can be cells in the central nervous system of a subject such as, for example, post-mitotic neurons in the brain. A target area or intended target area can be one or more of a subject's organs (e.g., lungs, heart, liver, kidney, brain, etc.). In an aspect, a target area or intended target area can be any cell or any organ infected by a genetic disease or disorder or a neurological disease or disorder. In an aspect, a target area or intended target area can be the brain or various neuron populations.

As used herein, “determining” can refer to measuring or ascertaining the presence and severity of a genetic disease or disorder or a neurological disease or disorder. Methods and techniques used to determine the presence and/or severity of a genetic disease or disorder or a neurological disease or disorder are typically known to the medical arts. For example, the art is familiar with the ways to identify and/or diagnose the presence, severity, or both of a genetic disease or disorder or a neurological disease or disorder. In an aspect, “determining” can also refer to measuring or ascertaining the level of expression or activity of one or more disclosed HDACs. In an aspect, “determining” can also refer to measuring or ascertaining a cells HDAC genotype, the packaging capacity of a generated optimized viral vector, the titer of a generated optimized viral vector, the expression capacity of a generated optimized viral vector, the immunogenicity and/or toxicity of a generated optimized viral vector, the ability of generated optimized viral vector to efficiently transduce cells, or any combination thereof.

As used herein, “effective amount” and “amount effective” can refer to an amount that is sufficient to achieve the desired result such as, for example, the treatment and/or prevention of a genetic disease or disorder or a neurological disease or disorder. As used herein, the terms “effective amount” and “amount effective” can refer to an amount that is sufficient to achieve the desired an effect on an undesired condition (e.g., a genetic disease or disorder or a neurological disease or disorder). For example, a “therapeutically effective amount” refers to an amount that is sufficient to achieve the desired therapeutic result or to have an effect on undesired symptoms but is generally insufficient to cause adverse side effects. In an aspect, “therapeutically effective amount” means an amount a disclosed isolated nucleic acid molecule, a disclosed shRNA targeting an HDAC, a disclosed viral vector, a disclosed producer cell, or a combination thereof, that (i) treats the particular disease, condition, or disorder (e.g., a genetic disease or disorder or a neurological disease or disorder), (ii) attenuates, ameliorates, or eliminates one or more symptoms of the particular disease, condition, or disorder (e.g., a genetic disease or disorder or a neurological disease or disorder), or (iii) delays the onset of one or more symptoms of the particular disease, condition, or disorder described herein (e.g., a genetic disease or disorder or a neurological disease or disorder). The specific therapeutically effective dose level for any particular patient will depend upon a variety of factors including the disorder being treated and the severity of the disorder; the disclosed isolated nucleic acid molecules, disclosed shRNAs targeting an HDAC, disclosed viral vectors, disclosed producer cells, or a combination thereof, employed; the disclosed methods employed; the age, body weight, general health, sex and diet of the patient; the time of administration; the route of administration; the rate of excretion of the disclosed isolated nucleic acid molecules, disclosed shRNAs targeting an HDAC, disclosed viral vectors, disclosed producer cells, or a combination thereof employed; the duration of the treatment; drugs used in combination or coincidental with the disclosed isolated nucleic acid molecules, disclosed shRNAs targeting an HDAC, disclosed viral vectors, disclosed producer cells, or a combination thereof employed, and other like factors well known in the medical arts. For example, it is well within the skill of the art to start doses of the disclosed isolated nucleic acid molecules, disclosed shRNAs targeting an HDAC, disclosed viral vectors, disclosed producer cells, or a combination thereof, at levels lower than those required to achieve the desired therapeutic effect and to gradually increase the dosage until the desired effect is achieved. If desired, then the effective daily dose can be divided into multiple doses for purposes of administration. Consequently, a single dose of the disclosed isolated nucleic acid molecule, a disclosed shRNA targeting an HDAC, a disclosed viral vector, a disclosed producer cell, or a combination thereof can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In an aspect, a preparation can be administered in a “prophylactically effective amount”; that is, an amount effective for prevention of a disease or condition, such as, for example, a genetic disease or disorder or a neurological disease or disorder.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents, or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. In an aspect, a pharmaceutical carrier employed can be a solid, liquid, or gas. In an aspect, examples of solid carriers can include lactose, terra alba, sucrose, talc, gelatin, agar, pectin, acacia, magnesium stearate, and stearic acid. In an aspect, examples of liquid carriers can include sugar syrup, peanut oil, olive oil, and water. In an aspect, examples of gaseous carriers can include carbon dioxide and nitrogen. In preparing a disclosed composition for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents.

Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “excipient” refers to an inert substance which is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, for reference, Remington's Pharmaceutical Sciences, (1990) Mack Publishing Co., Easton, Pa., which is hereby incorporated by reference in its entirety.

As used herein, the term “package insert” is used to refer to instructions customarily included in commercial packages of therapeutic products, that contain information about the indications, usage, dosage, administration, contraindications and/or warnings concerning the use of such therapeutic products.

As used herein, the term “in combination” in the context of the administration of one or more disclosed isolated nucleic acid molecules, disclosed shRNAs targeting an HDAC, disclosed viral vectors, disclosed producer cells, or a combination thereof includes the use of more than one therapy (e.g., additional therapeutic agents). Administration “in combination with” one or more additional therapeutic agents includes simultaneous (e.g., concurrent) and consecutive administration in any order. The use of the term “in combination” does not restrict the order in which therapies are administered to a subject. By way of non-limiting example, a first therapy (e.g., one or more of the disclosed agents, disclosed therapeutic agents, disclosed pharmaceutical formulations, or a combination thereof) can be administered prior to (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 8 weeks, 8 weeks, 9 weeks, 10 weeks, 11 weeks, or 12 weeks), concurrently, or after (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 7 weeks, 8 weeks, 9 weeks, 10 weeks, 11 weeks, or 12 weeks or longer) the administration of a second therapy (e.g., one or more disclosed isolated nucleic acid molecules, disclosed shRNAs targeting an HDAC, disclosed viral vectors, disclosed producer cells, or a combination thereof, or one or more additional therapeutic agents) to a subject having or diagnosed with a genetic disease or disorder or a neurological disease or disorder.

Disclosed are the components to be used to prepare the disclosed agents, disclosed therapeutic agents, and/or the disclosed pharmaceutical formulations as well the disclosed agents, disclosed therapeutic agents, and/or the disclosed pharmaceutical formulations used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific aspects or combination of aspects of the disclosed methods.

### B. Compositions for Use in Disclosed Methods

Disclosed herein are compositions for use in performing one or more disclosed methods. In an aspect, a disclosed composition can comprise a disclosed isolated nucleic acid molecule, a disclosed shRNA targeting a disclosed HDAC, a disclosed viral vector, a disclosed virus producer cell line, a disclosed cell transduced by a disclosed viral vector, or any combination thereof.

### 1. Isolated Nucleic Acid Molecules

Disclosed herein is an isolated nucleic acid molecule comprising a nucleic acid sequence encoding (i) a Cas endonuclease, (ii) at least one guide RNA, and (iii) at least one transgene.

Disclosed herein is an isolated nucleic acid molecule comprising a nucleic acid sequence encoding (i) a fusion product, and (ii) at least one guide RNA.

Disclosed herein is an isolated nucleic acid molecule comprising a nucleic acid sequence encoding (i) a Cas endonuclease, (ii) at least one guide RNA, (iii) at least one transgene, and (iv) shRNA targeting a histone deacetylase (HDAC).

Disclosed herein is an isolated nucleic acid molecule comprising a nucleic acid sequence encoding (i) a fusion product, (ii) at least one guide RNA, and (iii) and shRNA targeting a histone deacetylase (HDAC).

In an aspect, a disclosed Cas endonuclease can comprise Cas9, SpCas9, SaCas9, a variant Cas9, a dCas, or a dCas9. In an aspect, a disclosed encoded Cas9 can comprise the sequence set forth in SEQ ID NO:52 or SEQ ID NO:53. In an aspect, a disclosed encoded Cas9 can have a sequence having at least 75%, at least 80%, at least 85%, at least 90%, or at least 95% identity to the sequence set forth in SEQ ID NO:52 or SEQ ID NO:53 or a fragment thereof.

In an aspect, a disclosed variant Cas9 can comprise VQR, EQR, or VRER. In an aspect a disclosed VRER can comprise the sequence set forth in SEQ ID NO:55. In an aspect, a disclosed VRER can have a sequence having at least 75%, at least 80%, at least 85%, at least 90%, or at least 95% identity to the sequence set forth in SEQ ID NO:55 or a fragment thereof.

In an aspect, a disclosed dCas can comprise dVQR, dEQR, or dVRER. In an aspect, a disclosed encoded dCas can comprise the sequence set forth in SEQ ID NO:54. In an aspect, a disclosed encoded dCas9 can have a sequence having at least 75%, at least 80%, at least 85%, at least 90%, or at least 95% identity to the sequence set forth in SEQ ID NO:54 or a fragment thereof.

A SpCas9 (3′NGG-PAM sequence) can comprise SpCas9 VQR (3′NGAN or 3′NGNG), SpCas9 EQR (3′NGAG), or SpCas9 VRER (3′NGCG).

In an aspect, a disclosed isolated nucleic acid molecule can further comprise a nucleic acid sequence encoding one or more Sp1 binding sites, one or more NFκB binding sites, or any combination thereof. In an aspect, a disclosed isolated nucleic acid molecule can further comprise a nucleic acid sequence encoding one or more regulatory elements. In an aspect, a disclosed regulatory element can comprise one or more promoters, one or more primer binding sites (PBS), one or more splice donor (SD) sites, one or more splice acceptor (SA) sites, one or more central polypurine tracts (cPPT), one or more polypurine tracts (PPT), one or more Rev Response elements (RRE), one or more Woodchuck Hepatitis Virus Posttranscriptional Regulatory Elements (WPRE), one or more retroviral vector packaging elements, or any combination thereof. In an aspect, a disclosed retroviral vector packaging element can comprise a psi (W) signal. In an aspect, a disclosed promoter can comprise a human Cytomegalovirus (hCMV) promoter, a core-elongation factor 1a promoter (EFS), a human U6 promoter are also included, or any combination thereof. In an aspect, a disclosed isolated nucleic acid molecule can comprise a viral 3′ UTR and/or a viral 5′ UTR. In an aspect of a disclosed isolated nucleic acid molecule, the disclosed nucleic acid sequence can comprise a deletion of the U3 region in the viral 3′ LTR.

In an aspect, a disclosed isolated nucleic acid molecule can further comprise a polylinker site. In an aspect, a disclosed polylinker site can comprise a pair of BsmBI sites and a unique BsrGI site. In an aspect, a disclosed pair of BsmBI sites can be used for cloning of a sgRNA. In an aspect, a disclosed BsrGI site can be used for verification of a cloned sgRNA.

In an aspect, a disclosed nucleic acid molecule can comprise a sequence encoding a reporter gene. In an aspect, a disclosed reporter gene can be used for validation. Reporter genes are known to the art. For example, in an aspect, a reporter gene can be a gene encoding a protein that is detectable or has an activity that produces a detectable product. A reporter gene can encode a visual marker or enzyme that produces a detectable signal, such as cat, lacZ, udA, xyIE, an alkaline phosphatase gene, an a-amylase gene, an α-galactosidase gene, α-glucuronidase gene, a p lactamase gene, a horseradish peroxidase gene, a luciferin/luciferase gene, an R-locus gene, a tyrosinase gene, or a gene encoding a fluorescent protein, including but not limited to a blue, cyan, green, red, paprika or yellow fluorescent protein, a photoconvertible, photoswitchable, or optical highlighter fluorescent protein, or any of variant thereof, including, without limitation, codon-optimized, rapidly folding, monomeric, increased stability, and enhanced fluorescence variants.

In an aspect, a disclosed reporter can comprise GFP. In an aspect, a disclosed GFP can comprise the sequence set forth in SEQ ID NO:03. In an aspect, a disclosed encoded GFP can comprise the sequence set forth in SEQ ID NO:04.

In an aspect, a disclosed transgene can encode a polypeptide. In an aspect, a disclosed encoded polypeptide can comprise transcription activation activity, transcription repression activity, transcription release factor activity, histone modification activity, nucleic acid association activity, methyltransferase activity, demethylase activity, acetyltransferase activity, deacetylase activity, or any combination thereof. In an aspect, a disclosed encoded polypeptide can be histone deacetylase or heterochromatin protein 1. In an aspect, a disclosed encoded polypeptide can comprise transcription repression activity.

In an aspect, a disclosed transgene can comprise a neurodegenerative disease (NDD)-relevant gene. In an aspect, NDDs can comprise Parkinson's disease (PD), Dementia with Lewy Bodies (DLB), related synucleinopathies, Alzheimer's disease (AD), Amyotrophic lateral sclerosis (ALS), Huntington's disease (HD), or any combination thereof. In an aspect, an NDD-relevant gene can comprise APOE, APP, ATXN2, CHMP2B, DCTN1, FIG4, FUS, GBA, GRN, HNRNPA1, HTT, LRRK2, MATR3, OPTN, PARK7, PFN1, PRPH, PSEN1, SETX, SIGMAR1, SNCA, SOD1, SPG11, SQSTM1, TARDBP, TBK1, TBP, TRPM7, TUBA4A, UBQLN2, UCHL1, VAPB, VCP, VPS35, a fragment thereof, a variant thereof, or a chimera thereof.

In an aspect, a disclosed encoded polypeptide can be histone deactylase or heterochromatin protein 1. In an aspect, a disclosed encoded polypeptide can comprise transcription repression activity. In an aspect, a disclosed DNMT3A can have the amino acid sequence set forth in SEQ ID NO:60 or the nucleotide sequence set forth in SEQ ID NO:61. In an aspect, a disclosed DNMT3A can have a sequence having at least 75%, at least 80%, at least 85%, at least 90%, or at least 95% identity to the sequence set forth in SEQ ID NO:60 or SEQ ID NO:61 or a fragment thereof.

In an aspect, at least one encoded polypeptide can comprise Kruppel-associated box (KRAB), the transcription repression domain (TRD) of Methyl-CpG Binding Protein 2 (MeCP2), or a fusion of KRAB-MeCP2 (KRAB-MeCP2). In an aspect, a disclosed TRD of MeCP2 can comprise the nucleotide sequence set forth in SEQ ID NO:57 or the amino acid sequence set forth in SEQ ID NO:56. In an aspect, a disclosed TRD of MeCP2 can have a sequence having at least 75%, at least 80%, at least 85%, at least 90%, or at least 95% identity to the sequence set forth in SEQ ID NO:57 or SEQ ID NO:56 or a fragment thereof.

In an aspect, a disclosed KRAB-MeCP2 can comprise the nucleotide sequence set forth in SEQ ID NO:58 or the amino acid sequence set forth in SEQ ID NO:59. In an aspect, a disclosed KRAB-MeCP2 can have a sequence having at least 75%, at least 80%, at least 85%, at least 90%, or at least 95% identity to the sequence set forth in SEQ ID NO:58 or SEQ ID NO:59 or a fragment thereof.

In an aspect, a disclosed Cas endonuclease can be fused to a disclosed polypeptide. In an aspect, a disclosed Cas endonuclease can be fused to a disclosed polypeptide having an enzymatic activity. In an aspect, a disclosed fusion protein can encode a disclosed Cas endonuclease and a disclosed polypeptide. In an aspect, a disclosed fusion protein can encode a disclosed Cas endonuclease and a disclosed polypeptide having any enzymatic activity. In an aspect, a disclosed encoded fused polypeptide can comprise transcription activation activity, transcription repression activity, transcription release factor activity, histone modification activity, nucleic acid association activity, methyltransferase activity, demethylase activity, acetyltransferase activity, deacetylase activity, or any combination thereof.

In an aspect, a disclosed fusion protein can comprise dCas9 and DNMT3A. In an aspect, a dCas9-DNMT3A fusion protein can have the amino acid sequence set forth SEQ ID NO:62. In an aspect, a dCas9-DNMT3A fusion protein can be encoded by the sequence set forth in SEQ ID NO:63. In an aspect, a disclosed dCas9-DNMT3A fusion protein can have a sequence having at least 75%, at least 80%, at least 85%, at least 90%, or at least 95% identity to the sequence set forth in SEQ ID NO:62 or SEQ ID NO:63 or a fragment thereof.

In an aspect, a disclosed Cas endonuclease can be dVRER and the polypeptide can be DNMT3A. In an aspect, a disclosed dVRER-DNMT3A fusion protein can have the amino acid sequence set forth in SEQ ID NO:64. In an aspect, a disclosed dVRER-DNMT3A fusion protein can be encoded by the sequence set forth in SEQ ID NO:65. In an aspect, a disclosed dVRER-DNMT3A fusion protein can have a sequence having at least 75%, at least 80%, at least 85%, at least 90%, or at least 95% identity to the sequence set forth in SEQ ID NO:64 or SEQ ID NO:65 or a fragment thereof.

In an aspect, a disclosed fusion protein can comprise dCas9 and Kruppel-associated box (KRAB), the transcription repression domain (TRD) of Methyl-CpG Binding Protein 2 (MeCP2), or a fusion of KRAB-MeCP2 (KRAB-MeCP2). In an aspect, a disclosed fusion protein can comprise dVRER and Kruppel-associated box (KRAB), the transcription repression domain (TRD) of Methyl-CpG Binding Protein 2 (MeCP2), or a fusion of KRAB-MeCP2 (KRAB-MeCP2).

In an aspect, a disclosed gRNA can comprise a targeting sequence for DNA binding (at, for example, a gene of interest such as an NDD-relevant gene) and a scaffold sequence for endonuclease binding (e.g., Cas9 binding).

In an aspect, a disclosed gRNA can target a promoter region, a methylated region, or any combination thereof in a disclosed gene of interest (such as, for example, an NDD-relevant gene).

In an aspect, a disclosed isolated nucleic acid molecule can further comprise a nucleic acid sequence encoding a shRNA targeting a histone deacetylase (HDAC). In an aspect, a disclosed isolated nucleic acid molecule can further comprise a nucleic acid sequence encoding one or more shRNAs targeting one or more HDACs. In an aspect, a disclosed isolated nucleic acid sequence can encode one or more shRNAs targeting one or more HDACs. Histone deacetylases (HDACs) are enzymes that catalyze the removal of acetyl functional groups from the lysine residues of both histone and nonhistone proteins. In humans, there are 18 HDAC enzymes that use either zinc-dependent or NAD⁺-dependent mechanisms to deacetylate acetyl lysine substrates. HDACs are known to those skilled in the art.

In an aspect, a disclosed HDAC can comprise a Class I Rpd3-like protein, a Class II Hda1-like proteins, a Class III Sir2-like protein, or a Class IV protein. In an aspect, a disclosed Class I Rpd3-like protein can comprise HDAC1, HDAC2, HDAC3, HDAC8, or any combination thereof. In an aspect, a disclosed Class II Hda1-like protein can comprise HDAC4, HDAC5, HDAC6, HDAC7, HDAC9, HDAC10, or any combination thereof. In an aspect, a disclosed Class III Sir2-like protein can comprise SIRT1, SIRT2, SIRT3, SIRT4, SIRT5, SIRT6, SIRT7, or any combination thereof. In an aspect, a disclosed Class IV protein can comprise HDAC11.

In an aspect, a disclosed HDAC can comprise HDAC1, HDAC2, HDAC3, HDAC4, HDAC6, or HDAC8, or any combination thereof. In an aspect, a disclosed HDAC can be HDAC8.

In an aspect, the open reading frame (ORF) of a disclosed HDAC can comprise the sequence set forth in any one of SEQ ID NO:07-SEQ ID NO:12. In an aspect, the open reading frame (ORF) of a disclosed HDAC1 can comprise the sequence set forth in SEQ ID NO:07. In an aspect, the open reading frame (ORF) of a disclosed HDAC2 can comprise the sequence set forth in SEQ ID NO:08. In an aspect, the open reading frame (ORF) of a disclosed HDAC3 can comprise the sequence set forth in SEQ ID NO:09. In an aspect, the open reading frame (ORF) of a disclosed HDAC4 can comprise the sequence set forth in SEQ ID NO:10. In an aspect, the open reading frame (ORF) of a disclosed HDAC6 can comprise the sequence set forth in SEQ ID NO:11. In an aspect, the open reading frame (ORF) of a disclosed HDAC8 can comprise the sequence set forth in SEQ ID NO:12. In an aspect, the open reading frame (ORF) of a disclosed HDAC can comprise a sequence having at least 50%, at least 55%, at least 60%, at least 65%, at least 70%, at least 75%, at least 80%, at least 85%, at least 90%, at least 95%, or more than 95% identity to the sequence set forth in any one of SEQ ID NO:07-SEQ ID NO:12.

In an aspect, a disclosed encoded HDAC can comprise the sequence set forth in any one of SEQ ID NO:13-SEQ ID NO:18. In an aspect, a disclosed encoded HDAC1 can comprise the sequence set forth in SEQ ID NO:13. In an aspect, a disclosed encoded HDAC2 can comprise the sequence set forth in SEQ ID NO:14. In an aspect, a disclosed encoded HDAC3 can comprise the sequence set forth in SEQ ID NO:15. In an aspect, a disclosed encoded HDAC4 can comprise the sequence set forth in SEQ ID NO:16. In an aspect, a disclosed encoded HDAC6 can comprise the sequence set forth in SEQ ID NO:17. In an aspect, a disclosed encoded HDAC8 can comprise the sequence set forth in SEQ ID NO:18.

In an aspect, a disclosed encoded HDAC can comprise a sequence having at least 50%, at least 55%, at least 60%, at least 65%, at least 70%, at least 75%, at least 80%, at least 85%, at least 90%, at least 95%, or more than 95% identity to the sequence set forth in any one of SEQ ID NO:13-SEQ ID NO:18.

In an aspect, a disclosed shRNA can target a disclosed Class I Rpd3-like protein. In an aspect, a disclosed shRNA can target a disclosed Class II Hda1-like protein. In an aspect, a disclosed shRNA can target a disclosed Class III Sir2-like protein. In an aspect, a disclosed shRNA can target a disclosed Class IV protein.

In an aspect, a disclosed shRNA can target HDAC1, HDAC2, HDAC3, HDAC4, HDA6, HDAC8, or any combination thereof. In an aspect, a disclosed shRNA targeting HDAC1 can comprise the sequence set forth in SEQ ID NO:19, or can comprise a sequence having at least 50%, at least 55%, at least 60%, at least 65%, at least 70%, at least 75%, at least 80%, at least 85%, at least 90%, at least 95%, or more than 95% identity to the sequence set forth in SEQ ID NO:19.

In an aspect, a disclosed shRNA targeting HDAC2 can comprise the sequence set forth in SEQ ID NO:20, or can comprise a sequence having at least 50%, at least 55%, at least 60%, at least 65%, at least 70%, at least 75%, at least 80%, at least 85%, at least 90%, at least 95%, or more than 95% identity to the sequence set forth in SEQ ID NO:20.

In an aspect, a disclosed shRNA targeting HDAC3 can comprise the sequence set forth in SEQ ID NO:21, or can comprise a sequence having at least 50%, at least 55%, at least 60%, at least 65%, at least 70%, at least 75%, at least 80%, at least 85%, at least 90%, at least 95%, or more than 95% identity to the sequence set forth in SEQ ID NO:21.

In an aspect, a disclosed shRNA targeting HDAC4 can comprise the sequence set forth in SEQ ID NO:22, or can comprise a sequence having at least 50%, at least 55%, at least 60%, at least 65%, at least 70%, at least 75%, at least 80%, at least 85%, at least 90%, at least 95%, or more than 95% identity to the sequence set forth in SEQ ID NO:22.

In an aspect, a disclosed shRNA targeting HDAC6 can comprise the sequence set forth in SEQ ID NO:23, or can comprise a sequence having at least 50%, at least 55%, at least 60%, at least 65%, at least 70%, at least 75%, at least 80%, at least 85%, at least 90%, at least 95%, or more than 95% identity to the sequence set forth in SEQ ID NO:23.

In an aspect, a disclosed shRNA targeting HDAC8 can comprise the sequence set forth in SEQ ID NO:24, or can comprise a sequence having at least 50%, at least 55%, at least 60%, at least 65%, at least 70%, at least 75%, at least 80%, at least 85%, at least 90%, at least 95%, or more than 95% identity to the sequence set forth in SEQ ID NO:24.

In an aspect, a disclosed nucleic acid sequence encoding a shRNA targeting a HDAC can be obtained from clone TRCN0000195467, clone TRCN0000004819, clone TRCN0000194993, clone TRCN0000004832, clone TRCN0000004839, or TRCN0000004851, or any combination thereof.

In an aspect, a disclosed isolated nucleic acid molecule can be packaged within a viral vector (e.g., adeno-associated virus, integrase-deficient or integrase competent, or a recombinant version thereof). In an aspect, a disclosed isolated nucleic acid molecule can increase the packaging capacity of a viral vector, can increase the titer of a viral vector, can increase the expression capacity of a viral vector, can decrease the immunogenicity and/or toxicity of a viral vector, can increase the ability of a viral vector to efficiently transduce one or more cells, or any combination thereof.

In an aspect, a disclosed isolated nucleic acid molecule can be expressed in a cell.

In an aspect, cells to be transduced by a generated optimized viral vector can comprise dividing cells and/or non-dividing cells. Cells be transduced by a generated optimized viral vector can be mammalian cells such as, for example, human, equine, bovine, porcine, canine, feline, or rodent cells. In an aspect, cells to be transduced by a generated optimized viral vector can be in a subject. In an aspect, a subject can be a human subject. In an aspect, cells to be transduced by a generated optimized viral vector can be cells in the central nervous system of a subject such as, for example, post-mitotic neurons in the brain.

In an aspect, a disclosed isolated nucleic acid molecule can be a part of all-in-one gene therapy delivery system for in vivo use in a subject (such as, for example, a human being with a genetic disease or disorder or a neurological disease or disorder).

### 2. Viral Vectors

Disclosed herein is a viral vector comprising a disclosed isolated nucleic acid. Disclosed herein is a viral vector comprising an isolated nucleic acid molecule comprising a nucleic acid sequence encoding (i) a Cas endonuclease, (ii) at least one guide RNA, and (iii) at least one transgene. Disclosed herein is a viral vector comprising an isolated nucleic acid molecule comprising a nucleic acid sequence encoding (i) a fusion product, and (ii) at least one guide RNA.

Disclosed herein is a viral vector comprising an isolated nucleic acid molecule comprising a nucleic acid sequence encoding (i) a Cas endonuclease, (ii) at least one guide RNA, (iii) at least one transgene, and (iv) shRNA targeting a histone deacetylase (HDAC).

Disclosed herein is a viral vector comprising an isolated nucleic acid molecule comprising a nucleic acid sequence encoding (i) a fusion product, (ii) at least one guide RNA, and (iii) and shRNA targeting a histone deacetylase (HDAC).

In an aspect of a disclosed viral vector, a disclosed Cas endonuclease can comprise Cas9, SpCas9, SaCas9, a variant Cas9, a dCas, or a dCas9. In an aspect, a disclosed encoded Cas9 can comprise the sequence set forth in SEQ ID NO:52 or SEQ ID NO:53. In an aspect, a disclosed encoded Cas9 can have a sequence having at least 75%, at least 80%, at least 85%, at least 90%, or at least 95% identity to the sequence set forth in SEQ ID NO:52 or SEQ ID NO:53 or a fragment thereof.

In an aspect, a disclosed variant Cas9 can comprise VQR, EQR, or VRER. In an aspect a disclosed VRER can comprise the sequence set forth in SEQ ID NO:55. In an aspect, a disclosed VRER can have a sequence having at least 75%, at least 80%, at least 85%, at least 90%, or at least 95% identity to the sequence set forth in SEQ ID NO:55 or a fragment thereof.

In an aspect, a disclosed dCas can comprise dVQR, dEQR, or dVRER. In an aspect, a disclosed encoded dCas can comprise the sequence set forth in SEQ ID NO:54. In an aspect, a disclosed encoded dCas9 can have a sequence having at least 75%, at least 80%, at least 85%, at least 90%, or at least 95% identity to the sequence set forth in SEQ ID NO:16 or a fragment thereof.

A SpCas9 (3′NGG-PAM sequence) can comprise SpCas9 VQR (3′NGAN or 3′NGNG), SpCas9 EQR (3′NGAG), or SpCas9 VRER (3′NGCG).

In an aspect, a disclosed viral vector can further comprise a nucleic acid sequence encoding one or more Sp1 binding sites, one or more NFκB binding sites, or any combination thereof.

In an aspect, a disclosed viral vector can further comprise a nucleic acid sequence encoding one or more regulatory elements. In an aspect, a disclosed regulatory element can comprise one or more promoters, one or more primer binding sites (PBS), one or more splice donor (SD) sites, one or more splice acceptor (SA) sites, one or more central polypurine tracts (cPPT), one or more polypurine tracts (PPT), one or more Rev Response elements (RRE), one or more Woodchuck Hepatitis Virus Posttranscriptional Regulatory Elements (WPRE), one or more retroviral vector packaging elements, or any combination thereof.

In an aspect, a disclosed retroviral vector packaging element can comprise a psi (y) signal.

In an aspect, a disclosed promoter can comprise a human Cytomegalovirus (hCMV) promoter, a core-elongation factor 1a promoter (EFS), a human U6 promoter are also included, or any combination thereof. In an aspect, a disclosed viral vector can further comprise a viral 3′ UTR and/or a viral 5′ UTR. In an aspect, a disclosed viral vector can comprise a deletion of the U3 region in the viral 3′ LTR.

In an aspect, a disclosed viral vector can further comprise a polylinker site. In an aspect, a disclosed polylinker site can comprise a pair of BsmBI sites and a unique BsrGI site. In an aspect, a disclosed pair of BsmBI sites can be used for cloning of a sgRNA. In an aspect, a disclosed BsrGI site can be used for verification of a cloned sgRNA.

In an aspect, a disclosed viral vector can comprise a sequence encoding a reporter gene.

In an aspect, a disclosed reporter gene can be used for validation. Reporter genes are known to the art. For example, in an aspect, a reporter gene can be a gene encoding a protein that is detectable or has an activity that produces a detectable product. A reporter gene can encode a visual marker or enzyme that produces a detectable signal, such as cat, lacZ, udA, xyIE, an alkaline phosphatase gene, an a-amylase gene, an a-galactosidase gene, a-glucuronidase gene, a p lactamase gene, a horseradish peroxidase gene, a luciferin/luciferase gene, an R-locus gene, a tyrosinase gene, or a gene encoding a fluorescent protein, including but not limited to a blue, cyan, green, red, paprika or yellow fluorescent protein, a photoconvertible, photoswitchable, or optical highlighter fluorescent protein, or any of variant thereof, including, without limitation, codon-optimized, rapidly folding, monomeric, increased stability, and enhanced fluorescence variants. In an aspect, a disclosed reporter can comprise GFP. In an aspect, a disclosed GFP can comprise the sequence set forth in SEQ ID NO:03. In an aspect, a disclosed encoded GFP can comprise the sequence set forth in SEQ ID NO:04.

In an aspect of a disclosed viral vector, a disclosed transgene can encode a polypeptide. In an aspect, a disclosed encoded polypeptide can comprise transcription activation activity, transcription repression activity, transcription release factor activity, histone modification activity, nucleic acid association activity, methyltransferase activity, demethylase activity, acetyltransferase activity, deacetylase activity, or any combination thereof. In an aspect, a disclosed encoded polypeptide can be histone deacetylase or heterochromatin protein 1. In an aspect, a disclosed encoded polypeptide can comprise transcription repression activity.

In an aspect of a disclosed viral vector, a disclosed transgene can comprise a neurodegenerative disease (NDD)-relevant gene. In an aspect, NDDs can comprise Parkinson's disease (PD), Dementia with Lewy Bodies (DLB), related synucleinopathies, Alzheimer's disease (AD), Amyotrophic lateral sclerosis (ALS), Huntington's disease (HD), or any combination thereof. In an aspect, an NDD-relevant gene can comprise APOE, APP, ATXN2, CHMP2B, DCTN1, FIG4, FUS, GBA, GRN, HNRNPA1, HTT, LRRK2, MATR3, OPTN, PARK7, PFN1, PRPH, PSEN1, SETX, SIGMAR1, SNCA, SOD1, SPG11, SQSTM1, TARDBP, TBK1, TBP, TRPM7, TUBA4A, UBQLN2, UCHL1, VAPB, VCP, VPS35, a fragment thereof, a variant thereof, or a chimera thereof.

In an aspect of a disclosed viral vector, a disclosed encoded polypeptide can be histone deacetylase or heterochromatin protein 1. In an aspect, a disclosed encoded polypeptide can comprise transcription repression activity. In an aspect, a disclosed DNMT3A can have the amino acid sequence set forth in SEQ ID NO:60 or the nucleotide sequence set forth in SEQ ID NO:61. In an aspect, a disclosed DNMT3A can have a sequence having at least 75%, at least 80%, at least 85%, at least 90%, or at least 95% identity to the sequence set forth in SEQ ID NO:60 or SEQ ID NO:61 or a fragment thereof.

In an aspect, at least one encoded polypeptide can comprise Kruppel-associated box (KRAB), the transcription repression domain (TRD) of Methyl-CpG Binding Protein 2 (MeCP2), or a fusion of KRAB-MeCP2 (KRAB-MeCP2). In an aspect, a disclosed TRD of MeCP2 can comprise the nucleotide sequence set forth in SEQ ID NO:57 or the amino acid sequence set forth in SEQ ID NO:56. In an aspect, a disclosed TRD of MeCP2 can have a sequence having at least 75%, at least 80%, at least 85%, at least 90%, or at least 95% identity to the sequence set forth in SEQ ID NO:57 or SEQ ID NO:56 or a fragment thereof. In an aspect, a disclosed KRAB-MeCP2 can comprise the nucleotide sequence set forth in SEQ ID NO:58 or the amino acid sequence set forth in SEQ ID NO:59. In an aspect, a disclosed KRAB-MeCP2 can have a sequence having at least 75%, at least 80%, at least 85%, at least 90%, or at least 95% identity to the sequence set forth in SEQ ID NO:58 or SEQ ID NO:59 or a fragment thereof.

In an aspect of a disclosed viral vector, a disclosed Cas endonuclease can be fused to a disclosed polypeptide. In an aspect, a disclosed Cas endonuclease can be fused to a disclosed polypeptide having an enzymatic activity. In an aspect of a disclosed viral vector, a disclosed fusion protein can encode a disclosed Cas endonuclease and a disclosed polypeptide. In an aspect, a disclosed fusion protein can encode a disclosed Cas endonuclease and a disclosed polypeptide having any enzymatic activity. In an aspect, a disclosed encoded fused polypeptide can comprise transcription activation activity, transcription repression activity, transcription release factor activity, histone modification activity, nucleic acid association activity, methyltransferase activity, demethylase activity, acetyltransferase activity, deacetylase activity, or any combination thereof.

In an aspect, a disclosed fusion protein can comprise dCas9 and DNMT3A. In an aspect, a dCas9-DNMT3A fusion protein can have the amino acid sequence set forth SEQ ID NO:62. In an aspect, a dCas9-DNMT3A fusion protein can be encoded by the sequence set forth in SEQ ID NO:63. In an aspect, a disclosed dCas9-DNMT3A fusion protein can have a sequence having at least 75%, at least 80%, at least 85%, at least 90%, or at least 95% identity to the sequence set forth in SEQ ID NO:62 or SEQ ID NO:63 or a fragment thereof.

In an aspect, a disclosed Cas endonuclease can be dVRER and the polypeptide can be DNMT3A. In an aspect, a disclosed dVRER-DNMT3A fusion protein can have the amino acid sequence set forth in SEQ ID NO:64. In an aspect, a disclosed dVRER-DNMT3A fusion protein can be encoded by the sequence set forth in SEQ ID NO:65. In an aspect, a disclosed dVRER-DNMT3A fusion protein can have a sequence having at least 75%, at least 80%, at least 85%, at least 90%, or at least 95% identity to the sequence set forth in SEQ ID NO:64 or SEQ ID NO:65 or a fragment thereof.

In an aspect, a disclosed fusion protein can comprise dCas9 and Kruppel-associated box (KRAB), the transcription repression domain (TRD) of Methyl-CpG Binding Protein 2 (MeCP2), or a fusion of KRAB-MeCP2 (KRAB-MeCP2). In an aspect, a disclosed fusion protein can comprise dVRER and Kruppel-associated box (KRAB), the transcription repression domain (TRD) of Methyl-CpG Binding Protein 2 (MeCP2), or a fusion of KRAB-MeCP2 (KRAB-MeCP2).

In an aspect a disclosed viral vector, a disclosed gRNA can comprise a targeting sequence for DNA binding (at, for example, a gene of interest such as an NDD-relevant gene) and a scaffold sequence for endonuclease binding (e.g., Cas9 binding).

In an aspect, a disclosed gRNA can target a promoter region, a methylated region, or any combination thereof in a disclosed gene of interest (such as, for example, an NDD-relevant gene).

In an aspect, a disclosed viral vector can further comprise a nucleic acid sequence encoding a shRNA targeting a histone deacetylase (HDAC). In an aspect, a disclosed viral vector can further comprise a nucleic acid sequence encoding one or more shRNAs targeting one or more HDACs. In an aspect, a disclosed viral vector can encode one or more shRNAs targeting one or more HDACs. Histone deacetylases (HDACs) are enzymes that catalyze the removal of acetyl functional groups from the lysine residues of both histone and nonhistone proteins. In humans, there are 18 HDAC enzymes that use either zinc-dependent or NAD⁺-dependent mechanisms to deacetylate acetyl lysine substrates. HDACs are known to those skilled in the art.

In an aspect, a disclosed HDAC can comprise a Class I Rpd3-like protein, a Class II Hda1-like proteins, a Class III Sir2-like protein, or a Class IV protein. In an aspect, a disclosed Class I Rpd3-like protein can comprise HDAC1, HDAC2, HDAC3, HDAC8, or any combination thereof. In an aspect, a disclosed Class II Hda1-like protein can comprise HDAC4, HDAC5, HDAC6, HDAC7, HDAC9, HDAC10, or any combination thereof. In an aspect, a disclosed Class III Sir2-like protein can comprise SIRT1, SIRT2, SIRT3, SIRT4, SIRT5, SIRT6, SIRT7, or any combination thereof. In an aspect, a disclosed Class IV protein can comprise HDAC11.

In an aspect, a disclosed HDAC can comprise HDAC1, HDAC2, HDAC3, HDAC4, HDAC6, or HDAC8, or any combination thereof. In an aspect, a disclosed HDAC can be HDAC8.

In an aspect, the open reading frame (ORF) of a disclosed HDAC can comprise the sequence set forth in any one of SEQ ID NO:07-SEQ ID NO:12. In an aspect, the open reading frame (ORF) of a disclosed HDAC1 can comprise the sequence set forth in SEQ ID NO:07. In an aspect, the open reading frame (ORF) of a disclosed HDAC2 can comprise the sequence set forth in SEQ ID NO:08. In an aspect, the open reading frame (ORF) of a disclosed HDAC3 can comprise the sequence set forth in SEQ ID NO:09. In an aspect, the open reading frame (ORF) of a disclosed HDAC4 can comprise the sequence set forth in SEQ ID NO:10. In an aspect, the open reading frame (ORF) of a disclosed HDAC6 can comprise the sequence set forth in SEQ ID NO:11. In an aspect, the open reading frame (ORF) of a disclosed HDAC8 can comprise the sequence set forth in SEQ ID NO:12. In an aspect, the open reading frame (ORF) of a disclosed HDAC can comprise a sequence having at least 50%, at least 55%, at least 60%, at least 65%, at least 70%, at least 75%, at least 80%, at least 85%, at least 90%, at least 95%, or more than 95% identity to the sequence set forth in any one of SEQ ID NO:07-SEQ ID NO:12.

In an aspect, a disclosed encoded HDAC can comprise the sequence set forth in any one of SEQ ID NO:13-SEQ ID NO:18. In an aspect, a disclosed encoded HDAC1 can comprise the sequence set forth in SEQ ID NO:13. In an aspect, a disclosed encoded HDAC2 can comprise the sequence set forth in SEQ ID NO:14. In an aspect, a disclosed encoded HDAC3 can comprise the sequence set forth in SEQ ID NO:15. In an aspect, a disclosed encoded HDAC4 can comprise the sequence set forth in SEQ ID NO:16. In an aspect, a disclosed encoded HDAC6 can comprise the sequence set forth in SEQ ID NO:17. In an aspect, a disclosed encoded HDAC8 can comprise the sequence set forth in SEQ ID NO:18.

In an aspect, a disclosed encoded HDAC can comprise a sequence having at least 50%, at least 55%, at least 60%, at least 65%, at least 70%, at least 75%, at least 80%, at least 85%, at least 90%, at least 95%, or more than 95% identity to the sequence set forth in any one of SEQ ID NO:13-SEQ ID NO:18.

In an aspect of a disclosed viral vector, a disclosed shRNA can target a disclosed Class I Rpd3-like protein. In an aspect, a disclosed shRNA can target a disclosed Class II Hda1-like protein. In an aspect, a disclosed shRNA can target a disclosed Class III Sir2-like protein. In an aspect, a disclosed shRNA can target a disclosed Class IV protein.

In an aspect of a disclosed viral vector, a disclosed shRNA can target HDAC1, HDAC2, HDAC3, HDAC4, HDA6, HDAC8, or any combination thereof.

In an aspect, a disclosed shRNA targeting HDAC1 can comprise the sequence set forth in SEQ ID NO:19, or can comprise a sequence having at least 50%, at least 55%, at least 60%, at least 65%, at least 70%, at least 75%, at least 80%, at least 85%, at least 90%, at least 95%, or more than 95% identity to the sequence set forth in SEQ ID NO:19. In an aspect, a disclosed shRNA targeting HDAC2 can comprise the sequence set forth in SEQ ID NO:20, or can comprise a sequence having at least 50%, at least 55%, at least 60%, at least 65%, at least 70%, at least 75%, at least 80%, at least 85%, at least 90%, at least 95%, or more than 95% identity to the sequence set forth in SEQ ID NO:20. In an aspect, a disclosed shRNA targeting HDAC3 can comprise the sequence set forth in SEQ ID NO:21, or can comprise a sequence having at least 50%, at least 55%, at least 60%, at least 65%, at least 70%, at least 75%, at least 80%, at least 85%, at least 90%, at least 95%, or more than 95% identity to the sequence set forth in SEQ ID NO:21. In an aspect, a disclosed shRNA targeting HDAC4 can comprise the sequence set forth in SEQ ID NO:22, or can comprise a sequence having at least 50%, at least 55%, at least 60%, at least 65%, at least 70%, at least 75%, at least 80%, at least 85%, at least 90%, at least 95%, or more than 95% identity to the sequence set forth in SEQ ID NO:22. In an aspect, a disclosed shRNA targeting HDAC6 can comprise the sequence set forth in SEQ ID NO:23, or can comprise a sequence having at least 50%, at least 55%, at least 60%, at least 65%, at least 70%, at least 75%, at least 80%, at least 85%, at least 90%, at least 95%, or more than 95% identity to the sequence set forth in SEQ ID NO:23. In an aspect, a disclosed shRNA targeting HDAC8 can comprise the sequence set forth in SEQ ID NO:24, or can comprise a sequence having at least 50%, at least 55%, at least 60%, at least 65%, at least 70%, at least 75%, at least 80%, at least 85%, at least 90%, at least 95%, or more than 95% identity to the sequence set forth in SEQ ID NO:24.

In an aspect of a disclosed viral vector, a disclosed nucleic acid sequence encoding a shRNA targeting a HDAC can be obtained from clone TRCN0000195467, clone TRCN0000004819, clone TRCN0000194993, clone TRCN0000004832, clone TRCN0000004839, or TRCN0000004851, or any combination thereof.

In an aspect, a disclosed viral vector can contact one or more cells. In an aspect, a disclosed viral vector can transduce a cell line.

In an aspect, a disclosed viral vector can comprise an adeno-associated virus (AAV), an integrase-deficient lentivirus (IDLV), or integrase competent lentivirus (ICLV). Lentiviruses include bovine immunodeficiency virus (BIV), caprine arthritis encephalitis virus (CAEV), equine infectious anemia virus (EIAV), feline immunodeficiency virus (FIV), puma lentivirus (PLV), simian immunodeficiency virus (SIV), visna/maedi virus (VMV), human immunodeficiency virus 1 (HIV-1), and human immunodeficiency virus 2 (HIV-2), or a recombinant thereof, or a mutation therefor, or a variant thereof, or a fragment thereof.

In an aspect, a disclosed viral vector can transduce a cell line to generate an HDAC−/− genotype. In an aspect, a disclosed HDAC−/− cell line can be used to increase the packaging capacity of a generated optimized viral vector, can increase the titer of a generated optimized viral vector, can increase the expression capacity of a generated optimized viral vector, can decrease the immunogenicity and/or toxicity of a generated optimized viral vector, can increase the ability of a generated optimized viral vector to efficiently transduce one or more cells, or any combination thereof.

Disclosed herein is a viral vector comprising the sequence set forth in any one of SEQ ID NO:26-SEQ ID NO:31. Disclosed herein is a viral vector comprising a sequence having at least 50%, at least 55%, at least 60%, at least 65%, at least 70%, at least 75%, at least 80%, at least 85%, at least 90%, at least 95%, or more than 95% identity to the sequence set forth in any one of SEQ ID NO:26-SEQ ID NO:31.

Disclosed herein is a viral vector comprising the sequence set forth in SEQ ID NO:26 and comprising a disclosed transgene (such as, for example, a disclosed NDD-relevant gene).

Disclosed herein is a viral vector comprising the sequence set forth in SEQ ID NO:27 and comprising a disclosed transgene (such as, for example, a disclosed NDD-relevant gene).

Disclosed herein is a viral vector comprising the sequence set forth in SEQ ID NO:28 and comprising a disclosed transgene (such as, for example, a disclosed NDD-relevant gene).

Disclosed herein is a viral vector comprising the sequence set forth in SEQ ID NO:29 and comprising a disclosed transgene (such as, for example, a disclosed NDD-relevant gene).

Disclosed herein is a viral vector comprising the sequence set forth in SEQ ID NO:30 and comprising a disclosed transgene (such as, for example, a disclosed NDD-relevant gene).

Disclosed herein is a viral vector comprising the sequence set forth in SEQ ID NO:31 and comprising a disclosed transgene (such as, for example, a disclosed NDD-relevant gene).

Disclosed herein is a viral vector comprising the sequence set forth in any one of SEQ ID NO:26-SEQ ID NO:31, and one or more of the following transgenes: APOE, APP, ATXN2, CHMP2B, DCTN1, FIG4, FUS, GBA, GRN, HNRNPA1, HTT, LRRK2, MATR3, OPTN, PARK7, PFN1, PRPH, PSEN1, SETX, SIGMAR1, SNCA, SOD1, SPG11, SQSTM1, TARDBP, TBK1, TBP, TRPM7, TUBA4A, UBQLN2, UCHL1, VAPB, VCP, VPS35, a fragment thereof, a variant thereof, or a chimera thereof.

Disclosed herein is a viral vector comprising a sequence having at least 50%, at least 55%, at least 60%, at least 65%, at least 70%, at least 75%, at least 80%, at least 85%, at least 90%, at least 95%, or more than 95% identity to the sequence set forth in any one of SEQ ID NO:26-SEQ ID NO:31, and one or more of the following transgenes: APOE, APP, ATXN2, CHMP2B, DCTN1, FIG4, FUS, GBA, GRN, HNRNPA1, HTT, LRRK2, MATR3, OPTN, PARK7, PFN1, PRPH, PSEN1, SETX, SIGMAR1, SNCA, SOD1, SPG11, SQSTM1, TARDBP, TBK1, TBP, TRPM7, TUBA4A, UBQLN2, UCHL1, VAPB, VCP, VPS35, a fragment thereof, a variant thereof, or a chimera thereof.

Disclosed herein is a viral vector comprising the sequence set forth in SEQ ID NO:01. Disclosed herein is a viral vector comprising a sequence having at least 50%, at least 55%, at least 60%, at least 65%, at least 70%, at least 75%, at least 80%, at least 85%, at least 90%, at least 95%, or more than 95% identity to the sequence set forth in SEQ ID NO:01. Disclosed herein is a viral vector comprising the sequence set forth in SEQ ID NO:01 and comprising a disclosed transgene (such as, for example, a disclosed NDD-relevant gene). Disclosed herein is a viral vector comprising the sequence set forth in SEQ ID NO:01, and one or more of the following transgenes: APOE, APP, ATXN2, CHMP2B, DCTN1, FIG4, FUS, GBA, GRN, HNRNPA1, HTT, LRRK2, MATR3, OPTN, PARK7, PFN1, PRPH, PSEN1, SETX, SIGMAR1, SNCA, SOD1, SPG11, SQSTM1, TARDBP, TBK1, TBP, TRPM7, TUBA4A, UBQLN2, UCHL1, VAPB, VCP, VPS35, a fragment thereof, a variant thereof, or a chimera thereof.

Disclosed herein is a viral vector comprising a sequence having at least 50%, at least 55%, at least 60%, at least 65%, at least 70%, at least 75%, at least 80%, at least 85%, at least 90%, at least 95%, or more than 95% identity to the sequence set forth in SEQ ID NO:01, and one or more of the following transgenes: APOE, APP, ATXN2, CHMP2B, DCTN1, FIG4, FUS, GBA, GRN, HNRNPA1, HTT, LRRK2, MATR3, OPTN, PARK7, PFN1, PRPH, PSEN1, SETX, SIGMAR1, SNCA, SOD1, SPG11, SQSTM1, TARDBP, TBK1, TBP, TRPM7, TUBA4A, UBQLN2, UCHL1, VAPB, VCP, VPS35, a fragment thereof, a variant thereof, or a chimera thereof.

In an aspect, cells to be transduced by a generated optimized viral vector can comprise dividing cells and/or non-dividing cells. Cells be transduced by a generated optimized viral vector can be mammalian cells such as, for example, human, equine, bovine, porcine, canine, feline, or rodent cells. In an aspect, cells to be transduced by a generated optimized viral vector can be in a subject. In an aspect, a subject can be a human subject. In an aspect, cells to be transduced by a generated optimized viral vector can be cells in the central nervous system of a subject such as, for example, post-mitotic neurons in the brain.

In an aspect, a disclosed viral vector can be a part of all-in-one gene therapy delivery system for in vivo use in a subject (such as, for example, a human being with a genetic disease or disorder or a neurological disease or disorder).

Disclosed herein are optimized viral vectors made by a disclosed method of developing HDAC depleted cells.

3. shRNAs Targeting HDAC

Disclosed herein is an isolated nucleic acid molecule comprising a nucleic acid sequence encoding a shRNA targeting a histone deacetylase (HDAC). Disclosed herein is an isolated nucleic acid molecule comprising a nucleic acid sequence encoding one or more shRNAs targeting a histone deacetylase (HDAC). Disclosed herein is an isolated nucleic acid molecule comprising a nucleic acid sequence encoding one or more shRNAs targeting one or more histone deacetylases (HDAC).

In an aspect, a disclosed shRNA can target the open reading frame (ORF) of a disclosed HDAC such as, for example, an HDAC comprising the sequence set forth in any one of SEQ ID NO:07-SEQ ID NO:12. In an aspect, the open reading frame (ORF) of a disclosed HDAC1 can comprise the sequence set forth in SEQ ID NO:07. In an aspect, the open reading frame (ORF) of a disclosed HDAC2 can comprise the sequence set forth in SEQ ID NO:08. In an aspect, the open reading frame (ORF) of a disclosed HDAC3 can comprise the sequence set forth in SEQ ID NO:09. In an aspect, the open reading frame (ORF) of a disclosed HDAC4 can comprise the sequence set forth in SEQ ID NO:10. In an aspect, the open reading frame (ORF) of a disclosed HDAC6 can comprise the sequence set forth in SEQ ID NO:11. In an aspect, the open reading frame (ORF) of a disclosed HDAC8 can comprise the sequence set forth in SEQ ID NO:12.

In an aspect, a disclosed shRNA can target the open reading frame (ORF) of a disclosed HDAC such as, for example, an HDAC comprising a sequence having at least 50%, at least 55%, at least 60%, at least 65%, at least 70%, at least 75%, at least 80%, at least 85%, at least 90%, at least 95%, or more than 95% identity to the sequence set forth in any one of SEQ ID NO:07-SEQ ID NO:12.

In an aspect, a disclosed shRNA can target an encoded HDAC comprising the sequence set forth in any one of SEQ ID NO:13-SEQ ID NO:18. In an aspect, a disclosed encoded HDAC1 can comprise the sequence set forth in SEQ ID NO:13. In an aspect, a disclosed encoded HDAC2 can comprise the sequence set forth in SEQ ID NO:14. In an aspect, a disclosed encoded HDAC3 can comprise the sequence set forth in SEQ ID NO:15. In an aspect, a disclosed encoded HDAC4 can comprise the sequence set forth in SEQ ID NO:16. In an aspect, a disclosed encoded HDAC6 can comprise the sequence set forth in SEQ ID NO:17. In an aspect, a disclosed encoded HDAC8 can comprise the sequence set forth in SEQ ID NO:18.

In an aspect, a disclosed shRNA can target an encoded HDAC comprising a sequence having at least 50%, at least 55%, at least 60%, at least 65%, at least 70%, at least 75%, at least 80%, at least 85%, at least 90%, at least 95%, or more than 95% identity to the sequence set forth in any one of SEQ ID NO:13-SEQ ID NO:18. Histone deacetylases (HDACs) are enzymes that catalyze the removal of acetyl functional groups from the lysine residues of both histone and nonhistone proteins. In humans, there are 18 HDAC enzymes that use either zinc- or NAD⁺-dependent mechanisms to deacetylate acetyl lysine substrates.

In an aspect, a disclosed shRNA can target a disclosed Class I Rpd3-like protein. In an aspect, a disclosed shRNA can target a disclosed Class II Hda1-like protein. In an aspect, a disclosed shRNA can target a disclosed Class III Sir2-like protein. In an aspect, a disclosed shRNA can target a disclosed Class IV protein. In an aspect, a disclosed targeted HDAC can comprise a Class I Rpd3-like protein, a Class II Hda1-like proteins, a Class III Sir2-like protein, or a Class IV protein. In an aspect, a disclosed targeted Class I Rpd3-like protein can comprise HDAC1, HDAC2, HDAC3, HDAC8, or any combination thereof. In an aspect, a disclosed targeted Class II Hda1-like protein can comprise HDAC4, HDAC5, HDAC6, HDAC7, HDAC9, HDAC10, or any combination thereof. In an aspect, a disclosed targeted Class III Sir2-like protein can comprise SIRT1, SIRT2, SIRT3, SIRT4, SIRT5, SIRT6, SIRT7, or any combination thereof.

In an aspect, a disclosed targeted Class IV protein can comprise HDAC11. In an aspect, a disclosed HDAC can comprise HDAC1, HDAC2, HDAC3, HDAC4, HDAC6, or HDAC8. In an aspect, a disclosed shRNA can target HDAC1, HDAC2, HDAC3, HDAC4, HDAC6, HDAC8, or any combination thereof.

In an aspect, a disclosed shRNA targeting HDAC1 can comprise the sequence set forth in SEQ ID NO:19, or can comprise a sequence having at least 50%, at least 55%, at least 60%, at least 65%, at least 70%, at least 75%, at least 80%, at least 85%, at least 90%, at least 95%, or more than 95% identity to the sequence set forth in SEQ ID NO:19.

In an aspect, a disclosed shRNA targeting HDAC2 can comprise the sequence set forth in SEQ ID NO:20, or can comprise a sequence having at least 50%, at least 55%, at least 60%, at least 65%, at least 70%, at least 75%, at least 80%, at least 85%, at least 90%, at least 95%, or more than 95% identity to the sequence set forth in SEQ ID NO:20.

In an aspect, a disclosed shRNA targeting HDAC3 can comprise the sequence set forth in SEQ ID NO:21, or can comprise a sequence having at least 50%, at least 55%, at least 60%, at least 65%, at least 70%, at least 75%, at least 80%, at least 85%, at least 90%, at least 95%, or more than 95% identity to the sequence set forth in SEQ ID NO:21.

In an aspect, a disclosed shRNA targeting HDAC4 can comprise the sequence set forth in SEQ ID NO:22, or can comprise a sequence having at least 50%, at least 55%, at least 60%, at least 65%, at least 70%, at least 75%, at least 80%, at least 85%, at least 90%, at least 95%, or more than 95% identity to the sequence set forth in SEQ ID NO:22.

In an aspect, a disclosed shRNA targeting HDAC6 can comprise the sequence set forth in SEQ ID NO:23, or can comprise a sequence having at least 50%, at least 55%, at least 60%, at least 65%, at least 70%, at least 75%, at least 80%, at least 85%, at least 90%, at least 95%, or more than 95% identity to the sequence set forth in SEQ ID NO:23.

In an aspect, a disclosed shRNA targeting HDAC8 can comprise the sequence set forth in SEQ ID NO:24, or can comprise a sequence having at least 50%, at least 55%, at least 60%, at least 65%, at least 70%, at least 75%, at least 80%, at least 85%, at least 90%, at least 95%, or more than 95% identity to the sequence set forth in SEQ ID NO:24.

In an aspect, a disclosed a nucleic acid sequence encoding a shRNA targeting a histone deacetylase (HDAC) can be obtained from clone TRCN0000195467, clone TRCN0000004819, clone TRCN0000194993, clone TRCN0000004832, clone TRCN0000004839, or TRCN0000004851.

In an aspect, a disclosed shRNA targeting a disclosed HDAC can be incorporated into a disclosed isolated nucleic acid molecule. In an aspect, a disclosed shRNA targeting a disclosed HDAC can be incorporated into a disclosed isolated nucleic acid molecule comprising a nucleic acid sequence encoding (i) a Cas endonuclease, (ii) at least one guide RNA, and (iii) at least one transgene. In an aspect, a disclosed shRNA targeting a disclosed HDAC can be incorporated into a disclosed isolated nucleic acid molecule comprising a nucleic acid sequence encoding (i) a fusion product, and (ii) at least one guide RNA.

In an aspect, a disclosed a nucleic acid sequence encoding a shRNA targeting a disclosed HDAC can be packaged within a viral vector (e.g., adeno-associated virus, integrase-deficient or integrase competent lentivirus, or a recombinant version thereof).

In an aspect, a disclosed a nucleic acid sequence encoding a shRNA targeting a disclosed HDAC can be expressed in a cell.

In an aspect, a disclosed shRNA can be used to produce a HDAC^{-/-} genotype. In an aspect, a disclosed shRNA targeting a disclosed HDAC1 can be used to produce a HDAC1^{-/-} genotype. In an aspect, a disclosed shRNA targeting a disclosed HDAC2 can be used to produce a HDAC2^{-/-} genotype. In an aspect, a disclosed shRNA targeting a disclosed HDAC3 can be used to produce a HDAC3^{-/-} genotype. In an aspect, a disclosed shRNA targeting a disclosed HDAC4 can be used to produce a HDAC4^{-/-} genotype. In an aspect, a disclosed shRNA targeting a disclosed HDAC6 can be used to produce a HDAC6^{-/-} genotype. In an aspect, a disclosed shRNA targeting a disclosed HDAC8 can be used to produce a HDAC8^{-/-} genotype.

In an aspect, a disclosed shRNA can be used to produce a HDAC^{-/-} genotype in one or more cells. In an aspect, a disclosed shRNA can be used to produce a HDAC^{-/-} genotype in a cell line. In an aspect, a disclosed shRNA can be used to produce a HDAC^{-/-} genotype in one or more cells, wherein the HDAC^{-/-} cell line can be used to generate an optimized viral vector. In an aspect, a disclosed HDAC^{-/-} cell line can increase the packaging capacity of a generated optimized viral vector, can increase the titer of a generated optimized viral vector, can increase the expression capacity of a generated optimized viral vector, can decrease the immunogenicity and/or toxicity of a generated optimized viral vector, can increase the ability of a generated optimized viral vector to efficiently transduce one or more cells, or any combination thereof.

In an aspect, cells to be transduced by a generated optimized viral vector can comprise dividing cells and/or non-dividing cells. Cells be transduced by a generated optimized viral vector can be mammalian cells such as, for example, human, equine, bovine, porcine, canine, feline, or rodent cells. In an aspect, cells to be transduced by a generated optimized viral vector can be in a subject. In an aspect, a subject can be a human subject. In an aspect, cells to be transduced by a generated optimized viral vector can be cells in the central nervous system of a subject such as, for example, post-mitotic neurons in the brain.

In an aspect, a disclosed shRNA targeting a HDAC can be a part of all-in-one gene therapy delivery system for in vivo use in a subject (such as, for example, a human being with a genetic disease or disorder or a neurological disease or disorder).

### 4. Virus Producer Cells

Disclosed herein is a virus producer cell line transduced with a disclosed viral vector. Disclosed herein is a virus producer cell line comprising a HDAC^{-/-} genotype. HDACs are known to those skilled in the art. Disclosed herein is a virus producer cell line comprising a HDAC^{-/-} genotype. Disclosed herein is a virus producer cell line comprising a HDAC2^{-/-} genotype. Disclosed herein is a virus producer cell line comprising a HDAC3^{-/-} genotype. Disclosed herein is a virus producer cell line comprising a HDAC4^{-/-} genotype. Disclosed herein is a virus producer cell line comprising a HDAC6^{-/-} genotype. Disclosed herein is a virus producer cell line comprising a HDAC8^{-/-} genotype.

In an aspect, a disclosed virus producer cell line can be a mammalian cell line such as human cell line, an equine cell line, a bovine cell line, a porcine cell line, a canine cell line, a feline cell line, or a rodent cells cell line (e.g., rat or mouse). In an aspect, a disclosed virus producer cell line can comprise HEK cells (e.g., HEK 293T cells).

In an aspect, the disclosed HDAC^{-/-} genotype can be HDAC1^{-/-}, HDAC2^{-/-}, HDAC3^{-/-}, HDAC4^{-/-}, HDAC6^{-/-}, HDAC8^{-/-}, or any combination thereof. In an aspect, the disclosed HDAC-cells can be HDAC1^{-/-} but still express HDAC2, HDAC3, HDAC4, HDAC6, HDAC8, or any combination thereof. In an aspect, the disclosed HDAC^{-/-} cells can be HDAC2^{-/-} but still express HDAC1, HDAC3, HDAC4, HDAC6, HDAC8, or any combination thereof. In an aspect, the disclosed HDAC^{-/-} cells can be HDAC3^{-/-} but still express HDAC1, HDAC2, HDAC4, HDAC6, HDAC8, or any combination thereof. In an aspect, the disclosed HDAC^{-/-} cells can be HDAC4^{-/-} but still express HDAC1, HDAC2, HDAC3, HDAC6, HDAC8, or any combination thereof. In an aspect, the disclosed HDAC^{-/-} cells can be HDAC6^{-/-} but still express HDAC1, HDAC2, HDAC3, HDAC4, HDAC8, or any combination thereof. In an aspect, the disclosed HDAC^{-/-} cells can be HDAC8^{-/-} but still express HDAC1, HDAC2, HDAC3, HDAC4, HDAC6, or any combination thereof.

In an aspect, the disclosed HDAC^{-/-} cells can comprise a complete decrease and/or depletion in expression and/or activity (e.g., 100% or about 100%). In an aspect, the disclosed HDAC^{-/-} cells can comprise an incomplete decrease and/or depletion in expression and/or activity. In an aspect, an incomplete decrease and/or depletion in expression and/or activity can comprise at least 50%, at least 55%, at least 60%, at least 65%, at least 70%, at least 75%, at least 80%, at least 85%, at least 90%, at least 95%, or more than 95% decrease and/or depletion when compared to cells having normal or wildtype HDAC expression and/or activity.

Disclosed herein is a virus producer cell line transduced with a viral vector comprising an isolated nucleic acid molecule comprising a nucleic acid sequence encoding (i) a Cas endonuclease, (ii) at least one guide RNA, and (iii) at least one transgene.

Disclosed herein is a virus producer cell line transduced with a viral vector comprising an isolated nucleic acid molecule comprising a nucleic acid sequence encoding (i) a fusion product, and (ii) at least one guide RNA.

Disclosed herein is a virus producer cell line transduced with a viral vector comprising an isolated nucleic acid molecule comprising a nucleic acid sequence encoding (i) a fusion product, (ii) at least one guide RNA, and (iii) and shRNA targeting a histone deacetylase (HDAC).

Disclosed herein is a virus producer cell line transduced with a viral vector comprising an isolated nucleic acid molecule comprising a nucleic acid sequence encoding (i) a Cas endonuclease, (ii) at least one guide RNA, (iii) at least one transgene, and (iv) shRNA targeting a histone deacetylase (HDAC).

In an aspect of a disclosed viral vector, a disclosed Cas endonuclease can comprise Cas9, SpCas9, SaCas9, a variant Cas9, a dCas, or a dCas9. In an aspect, a disclosed encoded Cas9 can comprise the sequence set forth in SEQ ID NO:52 or SEQ ID NO:53. In an aspect, a disclosed encoded Cas9 can have a sequence having at least 75%, at least 80%, at least 85%, at least 90%, or at least 95% identity to the sequence set forth in SEQ ID NO:52 or SEQ ID NO:53 or a fragment thereof.

In an aspect, a disclosed variant Cas9 can comprise VQR, EQR, or VRER. In an aspect a disclosed VRER can comprise the sequence set forth in SEQ ID NO:55. In an aspect, a disclosed VRER can have a sequence having at least 75%, at least 80%, at least 85%, at least 90%, or at least 95% identity to the sequence set forth in SEQ ID NO:55 or a fragment thereof. In an aspect, a disclosed dCas can comprise dVQR, dEQR, or dVRER. In an aspect, a disclosed encoded dCas can comprise the sequence set forth in SEQ ID NO:54. In an aspect, a disclosed dCas9 can have a sequence having at least 75%, at least 80%, at least 85%, at least 90%, or at least 95% identity to the sequence set forth in SEQ ID NO:54 or a fragment thereof.

A SpCas9 (3′NGG-PAM sequence) can comprise SpCas9 VQR (3′NGAN or 3′NGNG), SpCas9 EQR (3′NGAG), or SpCas9 VRER (3′NGCG).

In an aspect, a disclosed viral vector can further comprise a nucleic acid sequence encoding one or more Sp1 binding sites, one or more NFκB binding sites, or any combination thereof.

In an aspect, a disclosed viral vector can further comprise a nucleic acid sequence encoding one or more regulatory elements. In an aspect, a disclosed regulatory element can comprise one or more promoters, one or more primer binding sites (PBS), one or more splice donor (SD) sites, one or more splice acceptor (SA) sites, one or more central polypurine tracts (cPPT), one or more polypurine tracts (PPT), one or more Rev Response elements (RRE), one or more Woodchuck Hepatitis Virus Posttranscriptional Regulatory Elements (WPRE), one or more retroviral vector packaging elements, or any combination thereof.

In an aspect, a disclosed retroviral vector packaging element can comprise a psi (y) signal.

In an aspect, a disclosed promoter can comprise a human Cytomegalovirus (hCMV) promoter, a core-elongation factor 1a promoter (EFS), a human U6 promoter are also included, or any combination thereof. In an aspect, a disclosed viral vector can further comprise a viral 3′ UTR and/or a viral 5′ UTR. In an aspect, a disclosed viral vector can comprise a deletion of the U3 region in the viral 3′ LTR.

In an aspect, a disclosed viral vector can further comprise a polylinker site. In an aspect, a disclosed polylinker site can comprise a pair of BsmBI sites and a unique BsrGI site. In an aspect, a disclosed pair of BsmBI sites can be used for cloning of a sgRNA. In an aspect, a disclosed BsrGI site can be used for verification of a cloned sgRNA.

In an aspect, a disclosed viral vector can comprise a sequence encoding a reporter gene.

In an aspect, a disclosed reporter gene can be used for validation. Reporter genes are known to the art. For example, in an aspect, a reporter gene can be a gene encoding a protein that is detectable or has an activity that produces a detectable product. A reporter gene can encode a visual marker or enzyme that produces a detectable signal, such as cat, lacZ, udA, xyIE, an alkaline phosphatase gene, an a-amylase gene, an a-galactosidase gene, a-glucuronidase gene, a p lactamase gene, a horseradish peroxidase gene, a luciferin/luciferase gene, an R-locus gene, a tyrosinase gene, or a gene encoding a fluorescent protein, including but not limited to a blue, cyan, green, red, paprika or yellow fluorescent protein, a photoconvertible, photoswitchable, or optical highlighter fluorescent protein, or any of variant thereof, including, without limitation, codon-optimized, rapidly folding, monomeric, increased stability, and enhanced fluorescence variants. In an aspect, a disclosed reporter can comprise GFP. In an aspect, a disclosed GFP can comprise the sequence set forth in SEQ ID NO:03. In an aspect, a disclosed encoded GFP can comprise the sequence set forth in SEQ ID NO: 04.

In an aspect of a disclosed viral vector, a disclosed transgene can encode a polypeptide. In an aspect, a disclosed encoded polypeptide can comprise transcription activation activity, transcription repression activity, transcription release factor activity, histone modification activity, nucleic acid association activity, methyltransferase activity, demethylase activity, acetyltransferase activity, deacetylase activity, or any combination thereof. In an aspect, a disclosed encoded polypeptide can be histone deacetylase or heterochromatin protein 1. In an aspect, a disclosed encoded polypeptide can comprise transcription repression activity.

In an aspect of a disclosed viral vector, a disclosed transgene can comprise a neurodegenerative disease (NDD)-relevant gene. In an aspect, NDDs can comprise Parkinson's disease (PD), Dementia with Lewy Bodies (DLB), related synucleinopathies, Alzheimer's disease (AD), Amyotrophic lateral sclerosis (ALS), Huntington's disease (HD), or any combination thereof. In an aspect, an NDD-relevant gene can comprise APOE, APP, ATXN2, CHMP2B, DCTN1, FIG4, FUS, GBA, GRN, HNRNPA1, HTT, LRRK2, MATR3, OPTN, PARK7, PFN1, PRPH, PSEN1, SETX, SIGMAR1, SNCA, SOD1, SPG11, SQSTM1, TARDBP, TBK1, TBP, TRPM7, TUBA4A, UBQLN2, UCHL1, VAPB, VCP, VPS35, a fragment thereof, a variant thereof, or a chimera thereof.

In an aspect of a disclosed viral vector, a disclosed encoded polypeptide can be histone deacetylase or heterochromatin protein 1. In an aspect, a disclosed encoded polypeptide can comprise transcription repression activity. In an aspect, a disclosed DNMT3A can have the amino acid sequence set forth in SEQ ID NO:60 or the nucleotide sequence set forth in SEQ ID NO:61. In an aspect, a disclosed DNMT3A can have a sequence having at least 75%, at least 80%, at least 85%, at least 90%, or at least 95% identity to the sequence set forth in SEQ ID NO:60 or SEQ ID NO:61 or a fragment thereof.

In an aspect, at least one encoded polypeptide can comprise Kruppel-associated box (KRAB), the transcription repression domain (TRD) of Methyl-CpG Binding Protein 2 (MeCP2), or a fusion of KRAB-MeCP2 (KRAB-MeCP2). In an aspect, a disclosed TRD of MeCP2 can comprise the nucleotide sequence set forth in SEQ ID NO:57 or the amino acid sequence set forth in SEQ ID NO:56. In an aspect, a disclosed TRD of MeCP2 can have a sequence having at least 75%, at least 80%, at least 85%, at least 90%, or at least 95% identity to the sequence set forth in SEQ ID NO:57 or SEQ ID NO:56 or a fragment thereof. In an aspect, a disclosed KRAB-MeCP2 can comprise the nucleotide sequence set forth in SEQ ID NO:58 or the amino acid sequence set forth in SEQ ID NO:59. In an aspect, a disclosed KRAB-MeCP2 can have a sequence having at least 75%, at least 80%, at least 85%, at least 90%, or at least 95% identity to the sequence set forth in SEQ ID NO:58 or SEQ ID NO:59 or a fragment thereof.

In an aspect of a disclosed viral vector, a disclosed Cas endonuclease can be fused to a disclosed polypeptide. In an aspect, a disclosed Cas endonuclease can be fused to a disclosed polypeptide having an enzymatic activity. In an aspect of a disclosed viral vector, a disclosed fusion protein can encode a disclosed Cas endonuclease and a disclosed polypeptide. In an aspect, a disclosed fusion protein can encode a disclosed Cas endonuclease and a disclosed polypeptide having any enzymatic activity. In an aspect, a disclosed encoded fused polypeptide can comprise transcription activation activity, transcription repression activity, transcription release factor activity, histone modification activity, nucleic acid association activity, methyltransferase activity, demethylase activity, acetyltransferase activity, deacetylase activity, or any combination thereof.

In an aspect, a disclosed fusion protein can comprise dCas9 and DNMT3A. In an aspect, a dCas9-DNMT3A fusion protein can have the amino acid sequence set forth SEQ ID NO:62. In an aspect, a dCas9-DNMT3A fusion protein can be encoded by the sequence set forth in SEQ ID NO:63. In an aspect, a disclosed dCas9-DNMT3A fusion protein can have a sequence having at least 75%, at least 80%, at least 85%, at least 90%, or at least 95% identity to the sequence set forth in SEQ ID NO:62 or SEQ ID NO:63 or a fragment thereof.

In an aspect, a disclosed Cas endonuclease can be dVRER and the polypeptide can be DNMT3A. In an aspect, a disclosed dVRER-DNMT3A fusion protein can have the amino acid sequence set forth SEQ ID NO:64. In an aspect, a disclosed dVRER-DNMT3A fusion protein can be encoded by the sequence set forth in SEQ ID NO:65. In an aspect, a disclosed dVRER-DNMT3A fusion protein can have a sequence having at least 75%, at least 80%, at least 85%, at least 90%, or at least 95% identity to the sequence set forth in SEQ ID NO:64 or SEQ ID NO:65 or a fragment thereof.

In an aspect, a disclosed fusion protein can comprise dCas9 and Kruppel-associated box (KRAB), the transcription repression domain (TRD) of Methyl-CpG Binding Protein 2 (MeCP2), or a fusion of KRAB-MeCP2 (KRAB-MeCP2). In an aspect, a disclosed fusion protein can comprise dVRER and Kruppel-associated box (KRAB), the transcription repression domain (TRD) of Methyl-CpG Binding Protein 2 (MeCP2), or a fusion of KRAB-MeCP2 (KRAB-MeCP2).

In an aspect a disclosed viral vector, a disclosed gRNA can comprise a targeting sequence for DNA binding (at, for example, a gene of interest such as an NDD-relevant gene) and a scaffold sequence for endonuclease binding (e.g., Cas9 binding). In an aspect, a disclosed gRNA can target a promoter region, a methylated region, or any combination thereof in a disclosed gene of interest (such as, for example, an NDD-relevant gene).

In an aspect, a disclosed viral vector can further comprise a nucleic acid sequence encoding a shRNA targeting a histone deacetylase (HDAC). In an aspect, a disclosed viral vector can further comprise a nucleic acid sequence encoding one or more shRNAs targeting one or more HDACs. In an aspect, a disclosed viral vector can encode one or more shRNAs targeting one or more HDACs. Histone deacetylases (HDACs) are enzymes that catalyze the removal of acetyl functional groups from the lysine residues of both histone and nonhistone proteins. In humans, there are 18 HDAC enzymes that use either zinc-dependent or NAD⁺-dependent mechanisms to deacetylate acetyl lysine substrates. HDACs are known to those skilled in the art.

In an aspect, a disclosed HDAC can comprise a Class I Rpd3-like protein, a Class II Hda1-like proteins, a Class III Sir2-like protein, or a Class IV protein. In an aspect, a disclosed Class I Rpd3-like protein can comprise HDAC1, HDAC2, HDAC3, HDAC8, or any combination thereof. In an aspect, a disclosed Class II Hda1-like protein can comprise HDAC4, HDAC5, HDAC6, HDAC7, HDAC9, HDAC10, or any combination thereof. In an aspect, a disclosed Class III Sir2-like protein can comprise SIRT1, SIRT2, SIRT3, SIRT4, SIRT5, SIRT6, SIRT7, or any combination thereof. In an aspect, a disclosed Class IV protein can comprise HDAC11.

In an aspect, a disclosed HDAC can comprise HDAC1, HDAC2, HDAC3, HDAC4, HDAC6, or HDAC8, or any combination thereof. In an aspect, a disclosed HDAC can be HDAC8.

In an aspect, the open reading frame (ORF) of a disclosed HDAC can comprise the sequence set forth in any one of SEQ ID NO:07-SEQ ID NO:12. In an aspect, the open reading frame (ORF) of a disclosed HDAC1 can comprise the sequence set forth in SEQ ID NO:07. In an aspect, the open reading frame (ORF) of a disclosed HDAC2 can comprise the sequence set forth in SEQ ID NO:08. In an aspect, the open reading frame (ORF) of a disclosed HDAC3 can comprise the sequence set forth in SEQ ID NO:09. In an aspect, the open reading frame (ORF) of a disclosed HDAC4 can comprise the sequence set forth in SEQ ID NO:10. In an aspect, the open reading frame (ORF) of a disclosed HDAC6 can comprise the sequence set forth in SEQ ID NO:11. In an aspect, the open reading frame (ORF) of a disclosed HDAC8 can comprise the sequence set forth in SEQ ID NO:12. In an aspect, the open reading frame (ORF) of a disclosed HDAC can comprise a sequence having at least 50%, at least 55%, at least 60%, at least 65%, at least 70%, at least 75%, at least 80%, at least 85%, at least 90%, at least 95%, or more than 95% identity to the sequence set forth in any one of SEQ ID NO:07-SEQ ID NO:12.

In an aspect, a disclosed encoded HDAC can comprise the sequence set forth in any one of SEQ ID NO:13-SEQ ID NO:18. In an aspect, a disclosed encoded HDAC1 can comprise the sequence set forth in SEQ ID NO:13. In an aspect, a disclosed encoded HDAC2 can comprise the sequence set forth in SEQ ID NO:14. In an aspect, a disclosed encoded HDAC3 can comprise the sequence set forth in SEQ ID NO:15. In an aspect, a disclosed encoded HDAC4 can comprise the sequence set forth in SEQ ID NO:16. In an aspect, a disclosed encoded HDAC6 can comprise the sequence set forth in SEQ ID NO:17. In an aspect, a disclosed encoded HDAC8 can comprise the sequence set forth in SEQ ID NO:18.

In an aspect, a disclosed encoded HDAC can comprise a sequence having at least 50%, at least 55%, at least 60%, at least 65%, at least 70%, at least 75%, at least 80%, at least 85%, at least 90%, at least 95%, or more than 95% identity to the sequence set forth in any one of SEQ ID NO:13-SEQ ID NO:18.

In an aspect of a disclosed viral vector, a disclosed shRNA can target a disclosed Class I Rpd3-like protein. In an aspect, a disclosed shRNA can target a disclosed Class II Hda1-like protein. In an aspect, a disclosed shRNA can target a disclosed Class III Sir2-like protein. In an aspect, a disclosed shRNA can target a disclosed Class IV protein.

In an aspect of a disclosed viral vector, a disclosed shRNA can target HDAC1, HDAC2, HDAC3, HDAC4, HDA6, HDAC8, or any combination thereof.

In an aspect, a disclosed shRNA targeting HDAC1 can comprise the sequence set forth in SEQ ID NO:19, or can comprise a sequence having at least 50%, at least 55%, at least 60%, at least 65%, at least 70%, at least 75%, at least 80%, at least 85%, at least 90%, at least 95%, or more than 95% identity to the sequence set forth in SEQ ID NO:19. In an aspect, a disclosed shRNA targeting HDAC2 can comprise the sequence set forth in SEQ ID NO:20, or can comprise a sequence having at least 50%, at least 55%, at least 60%, at least 65%, at least 70%, at least 75%, at least 80%, at least 85%, at least 90%, at least 95%, or more than 95% identity to the sequence set forth in SEQ ID NO:20. In an aspect, a disclosed shRNA targeting HDAC3 can comprise the sequence set forth in SEQ ID NO:21, or can comprise a sequence having at least 50%, at least 55%, at least 60%, at least 65%, at least 70%, at least 75%, at least 80%, at least 85%, at least 90%, at least 95%, or more than 95% identity to the sequence set forth in SEQ ID NO:21. In an aspect, a disclosed shRNA targeting HDAC4 can comprise the sequence set forth in SEQ ID NO:22, or can comprise a sequence having at least 50%, at least 55%, at least 60%, at least 65%, at least 70%, at least 75%, at least 80%, at least 85%, at least 90%, at least 95%, or more than 95% identity to the sequence set forth in SEQ ID NO:22. In an aspect, a disclosed shRNA targeting HDAC6 can comprise the sequence set forth in SEQ ID NO:23, or can comprise a sequence having at least 50%, at least 55%, at least 60%, at least 65%, at least 70%, at least 75%, at least 80%, at least 85%, at least 90%, at least 95%, or more than 95% identity to the sequence set forth in SEQ ID NO:23. In an aspect, a disclosed shRNA targeting HDAC8 can comprise the sequence set forth in SEQ ID NO:24, or can comprise a sequence having at least 50%, at least 55%, at least 60%, at least 65%, at least 70%, at least 75%, at least 80%, at least 85%, at least 90%, at least 95%, or more than 95% identity to the sequence set forth in SEQ ID NO:24.

In an aspect of a disclosed viral vector, a disclosed nucleic acid sequence encoding a shRNA targeting a HDAC can be obtained from clone TRCN0000195467, clone TRCN0000004819, clone TRCN0000194993, clone TRCN0000004832, clone TRCN0000004839, or TRCN0000004851, or any combination thereof.

In an aspect, a disclosed viral vector can contact one or more cells. In an aspect, a disclosed viral vector can transduce a cell line to produce a disclosed virus producer cells or a disclosed virus producer cell line.

In an aspect, a disclosed viral vector can transduce a cell line to generate an HDAC^{-/-} genotype. In an aspect, a disclosed HDAC^{-/-} cell line can be used to increase the packaging capacity of a generated optimized viral vector, can increase the titer of a generated optimized viral vector, can increase the expression capacity of a generated optimized viral vector, can decrease the immunogenicity and/or toxicity of a generated optimized viral vector, can increase the ability of a generated optimized viral vector to efficiently transduce one or more cells, or any combination thereof.

In an aspect, a disclosed virus producer cell can be used to increase the packaging capacity of a generated optimized viral vector, can increase the titer of a generated optimized viral vector, can increase the expression capacity of a generated optimized viral vector, can decrease the immunogenicity and/or toxicity of a generated optimized viral vector, can increase the ability of a generated optimized viral vector to efficiently transduce one or more cells, or any combination thereof.

In an aspect, a disclosed virus producer cell line can generate an optimized viral vector.

In an aspect, a disclosed virus producer cell line can increase the packaging capacity of a generated optimized viral vector, can increase the titer of a generated optimized viral vector, can increase the expression capacity of a generated optimized viral vector, can decrease the immunogenicity and/or toxicity of a generated optimized viral vector, can increase the ability of a generated optimized viral vector to efficiently transduce one or more cells, or any combination thereof.

In an aspect, a disclosed generated optimized viral vector can have an increased titer, an increased packaging capacity, an increased expression capacity, a decreased immunogenicity and/or toxicity, an increased transduction efficiency, or any combination thereof when compared to a viral vector produced by a virus producer cell having normal or wildtype HDAC expression and/or activity.

In an aspect, a disclosed viral vector can comprise an adeno-associated virus (AAV), an integrase-deficient lentivirus (IDLV), or integrase competent lentivirus (ICLV). Lentiviruses include bovine immunodeficiency virus (BIV), caprine arthritis encephalitis virus (CAEV), equine infectious anemia virus (EIAV), feline immunodeficiency virus (FIV), puma lentivirus (PLV), simian immunodeficiency virus (SIV), visna/maedi virus (VMV), human immunodeficiency virus 1 (HIV-1), and human immunodeficiency virus 2 (HIV-2), or a recombinant thereof, or a mutation therefor, or a variant thereof, or a fragment thereof.

In an aspect, a disclosed generated optimized viral vector can comprise an adeno-associated virus (AAV), an integrase-deficient lentivirus (IDLV), or integrase competent lentivirus (ICLV). Lentiviruses include bovine immunodeficiency virus (BIV), caprine arthritis encephalitis virus (CAEV), equine infectious anemia virus (EIAV), feline immunodeficiency virus (FIV), puma lentivirus (PLV), simian immunodeficiency virus (SIV), visna/maedi virus (VMV), human immunodeficiency virus 1 (HIV-1), and human immunodeficiency virus 2 (HIV-2), or a recombinant thereof, or a mutation therefor, or a variant thereof, or a fragment thereof.

In an aspect, a disclosed virus producer cell can be generated using a disclosed isolated viral vector.

In an aspect, a disclosed virus producer cell can be used to generate a disclosed viral vector. In an aspect, a disclosed virus producer cell can be used to generate a viral vector having increased packaging capacity, increased titer, increased expression capacity, decreased immunogenicity and/or toxicity, and an increased ability to efficiently transduce cells.

In an aspect, cells to be transduced by a generated optimized viral vector can comprise dividing cells and/or non-dividing cells. Cells be transduced by a generated optimized viral vector can be mammalian cells such as, for example, human, equine, bovine, porcine, canine, feline, or rodent cells. In an aspect, cells to be transduced by a generated optimized viral vector can be in a subject. In an aspect, a subject can be a human subject. In an aspect, cells to be transduced by a generated optimized viral vector can be cells in the central nervous system of a subject such as, for example, post-mitotic neurons in the brain.

In an aspect, a disclosed virus producer cell can be a part of all-in-one gene therapy delivery system for in vivo use in a subject (such as, for example, a human being with a genetic disease or disorder or a neurological disease or disorder).

### 5. Kits

Disclosed herein is a kit comprising one or more disclosed compositions. Disclosed herein is a kit comprising a disclosed composition for a use in a disclosed method. Disclosed herein is a kit for use in a disclosed method of developing HDAC depleted cells. Disclosed herein is a kit for use in a disclosed method of increasing the packaging capacity of a viral vector. Disclosed herein is a kit for use in a disclosed method of increasing the titer of a viral vector. Disclosed herein is a kit for use in a disclosed method of increasing the expression capacity of a viral vector.

Disclosed herein is a kit for use in a disclosed method of decreasing the immunogenicity and/or toxicity of a viral vector. Disclosed herein is a kit for use in a disclosed method of increasing the ability of a viral vector to efficiently transduce one or more cells.

Disclosed herein is a kit comprising the reagents necessary to perform one or more of the disclosed methods. Disclosed herein is a kit comprising the disclosed compositions necessary to perform one or more of the disclosed methods. Disclosed herein is a kit comprising one or more of the disclosed isolated nucleic acid molecules, disclosed shRNAs targeting an HDAC, disclosed viral vectors, disclosed producer cells, or a combination thereof.

In an aspect, a disclosed kit can comprise cells or a cell line. In an aspect, disclosed cells or a disclosed cell line can comprise HEK cells (e.g., HEK 293 cells, HEK 293T, HEK 293FT, HEK 293S, HEK 293FT, HEK 293SG, HEK 293SGGD, HEK 293H, HEK 293E, HEK 293A, HEK 293MSR, etc.).

In an aspect, a disclosed kit can comprise at least two components constituting the kit. Together, the components constitute a functional unit for a given purpose (such as, for example, developing HDAC depleted cells). Individual member components can be physically packaged together or separately. For example, a kit comprising an instruction for using the kit may or may not physically include the instruction with other individual member components. Instead, the instruction can be supplied as a separate member component, either in a paper form or an electronic form which can be supplied on computer readable memory device or downloaded from an internet website, or as recorded presentation. In an aspect, a kit for use in a disclosed method can comprise one or more containers holding a disclosed isolated nucleic acid molecule, a disclosed shRNA targeting an HDAC, a disclosed viral vector, a disclosed producer cell, or a combination thereof, and a label or package insert with instructions for use. In an aspect, suitable containers include, for example, bottles, vials, syringes, blister pack, etc. The containers can be formed from a variety of materials such as glass or plastic. The container can hold, for example, a disclosed isolated nucleic acid molecule, a disclosed shRNA targeting an HDAC, a disclosed viral vector, a disclosed producer cell, or a combination thereof, and can have a sterile access port (for example the container can be an intravenous solution bag or a vial having a stopper pierceable by a hypodermic injection needle). The label or package insert can indicate that a disclosed isolated nucleic acid molecule, a disclosed shRNA targeting an HDAC, a disclosed viral vector, a disclosed producer cell, or a combination thereof, can be used for treating, preventing, inhibiting, and/or ameliorating a genetic disease or disorder or a neurological disease or disorder. In an aspect, a disclosed kit can comprise additional components necessary for administration such as, for example, other buffers, diluents, filters, needles, and syringes.

### C. Disclosed Methods

### 1. Methods of Developing HDAC Depleted Cells

Disclosed herein is a method of developing HDAC depleted cells, the method comprising contacting one or more cells with a disclosed viral vector; wherein following transduction of the one or more cells, the expression and/or activity of the targeted HDAC is decreased and/or depleted. Disclosed herein is a method of developing HDAC depleted cells, the method comprising contacting one or more cells with a viral vector comprising an isolated nucleic acid molecule comprising a nucleic acid sequence encoding (i) a Cas endonuclease, (ii) at least one guide RNA, and (iii) at least one transgene; wherein following transduction of the one or more cells, the expression and/or activity of the targeted HDAC is decreased and/or depleted. Disclosed herein is a method of developing HDAC depleted cells, the method comprising contacting one or more cells with a viral vector comprising an isolated nucleic acid molecule comprising a nucleic acid sequence encoding (i) a fusion product, and (ii) at least one guide RNA; wherein following transduction of the one or more cells, the expression and/or activity of the targeted HDAC is decreased and/or depleted. Disclosed viral vectors are discussed supra.

In an aspect, the disclosed transduced cells can comprise a mammalian cell line such as human cell line, an equine cell line, a bovine cell line, a porcine cell line, a canine cell line, a feline cell line, or a rodent cells cell line (e.g., rat or mouse). In an aspect, the disclosed transduced cell line can comprise HEK cells (e.g., HEK 293 cells, HEK 293T, HEK 293FT, HEK 293S, HEK 293FT, HEK 293SG, HEK 293SGGD, HEK 293H, HEK 293E, HEK 293A, HEK 293MSR, etc.).

In an aspect, the disclosed HDAC^{-/-} cells can increase the packaging capacity of a generated optimized viral vector. In an aspect, the disclosed HDAC^{-/-} cells can increase the titer of a generated optimized viral vector. In an aspect, the disclosed HDAC^{-/-} cells can increase the expression capacity of a generated optimized viral vector. In an aspect, the disclosed HDAC^{-/-} cells can decrease the immunogenicity and/or toxicity of a generated optimized viral vector. In an aspect, the disclosed HDAC^{-/-} cells can increase the ability of the generated optimized viral vector to efficiently transduce one or more cells.

In an aspect, the disclosed HDAC^{-/-} cells can increase the packaging capacity of a generated optimized viral vector, increase the titer of a generated optimized viral vector, increase the expression capacity of a generated optimized viral vector, decrease the immunogenicity and/or toxicity of a generated optimized viral vector, increase the ability of a generated optimized viral vector to efficiently transduce one or more cells, or any combination thereof when compared to cells having normal or wildtype HDAC expression and/or activity.

In an aspect, the disclosed HDAC^{-/-} cells can be HDAC1^{-/-}, HDAC2^{-/-}, HDAC3^{-/-}, HDAC4^{-/-}, HDAC6^{-/-}, HDAC8^{-/-}, or any combination thereof. In an aspect, the disclosed HDAC^{-/-} cells can be HDAC1^{-/-} but still express HDAC2, HDAC3, HDAC4, HDAC6, HDAC8, or any combination thereof. In an aspect, the disclosed HDAC^{-/-} cells can be HDAC2^{-/-} but still express HDAC1, HDAC3, HDAC4, HDAC6, HDAC8, or any combination thereof. In an aspect, the disclosed HDAC^{-/-} cells can be HDAC3^{-/-} but still express HDAC1, HDAC2, HDAC4, HDAC6, HDAC8, or any combination thereof. In an aspect, the disclosed HDAC^{-/-} cells can be HDAC4^{-/-} but still express HDAC1, HDAC2, HDAC3, HDAC6, HDAC8, or any combination thereof. In an aspect, the disclosed HDAC^{-/-} cells can be HDAC67—but still express HDAC1, HDAC2, HDAC3, HDAC4, HDAC8, or any combination thereof. In an aspect, the disclosed HDAC^{-/-} cells can be HDAC8^{-/-} but still express HDAC1, HDAC2, HDAC3, HDAC4, HDAC6, or any combination thereof.

In an aspect, the disclosed HDAC^{-/-} cells can comprise a complete decrease and/or depletion in expression and/or activity (e.g., 100% or about 100%). In an aspect, the disclosed HDAC^{-/-} cells can comprise an incomplete decrease and/or depletion in expression and/or activity. In an aspect, an incomplete decrease and/or depletion in expression and/or activity can comprise at least 50%, at least 55%, at least 60%, at least 65%, at least 70%, at least 75%, at least 80%, at least 85%, at least 90%, at least 95%, or more than 95% decrease and/or depletion when compared to cells having normal or wildtype HDAC expression and/or activity.

In an aspect, the disclosed HDAC^{-/-} cells can be used to generate a ICLV vector or an IDLV vector. In an aspect, the disclosed HDAC^{-/-} cells can be used to generate an AAV vector or rAAV vector. In an aspect, the disclosed HDAC depleted cells can generate a viral vector that is compatible with advanced manufacturing techniques.

In an aspect, the disclosed HDAC^{-/-} cells can generate a viral vector that can efficiently transduce dividing and/or non-dividing cells (e.g., such as, for example, postmitotic neurons of the brain).

In an aspect of a method of developing HDAC depleted cells, the method can further comprise administering a therapeutically effective amount of the generated optimized viral vector to a subject in need thereof. In an aspect, a subject in need thereof can comprise a subject having a genetic defect or disorder. For example, in an aspect, a subject can have a genetic defect or disorder that affects an NDD-related gene.

In an aspect, a disclosed method of developing HDAC depleted cells can be a part of all-in-one gene therapy delivery system for in vivo use in a subject (such as, for example, a human being with a genetic disease or disorder or a neurological disease or disorder).

In an aspect, a disclosed method can further comprise determining the HDAC genotype of one or more cells, the packaging capacity of a generated optimized viral vector, the titer of a generated optimized viral vector, the expression capacity of a generated optimized viral vector, the immunogenicity and/or toxicity of a generated optimized viral vector, the ability of generated optimized viral vector to efficiently transduce cells, or any combination thereof.

### 2. Methods of Increasing the Packaging Capacity of a Viral Vector

Disclosed herein is a method of increasing the packaging capacity of a viral vector, the method comprising contacting one or more cells with a disclosed viral vector; wherein following transduction of the one or more cells, the expression and/or activity of the targeted HDAC is decreased and/or depleted; and wherein the generated optimized viral vector has an increased packaging capacity.

Disclosed herein is a method of increasing the packaging capacity of a viral vector, the method comprising contacting one or more cells with a viral vector comprising an isolated nucleic acid molecule comprising a nucleic acid sequence encoding (i) a Cas endonuclease, (ii) at least one guide RNA, and (iii) at least one transgene; wherein following transduction of the one or more cells, the expression and/or activity of the targeted HDAC is decreased and/or depleted; and wherein the generated optimized viral vector has an increased packaging capacity. Viral vectors are disclosed supra.

Disclosed herein is a method of increasing the packaging capacity of a viral vector, the method comprising contacting one or more cells with a viral vector comprising an isolated nucleic acid molecule comprising a nucleic acid sequence encoding (i) a fusion product, and (ii) at least one guide RNA; wherein following transduction of the one or more cells, the expression and/or activity of the targeted HDAC is decreased and/or depleted; and wherein the generated optimized viral vector has an increased packaging capacity. Viral vectors are disclosed supra.

Disclosed herein is a method of increasing the packaging capacity of a viral vector, the method comprising contacting one or more cells with a viral vector comprising an isolated nucleic acid molecule comprising a nucleic acid sequence encoding (i) a Cas endonuclease, (ii) at least one guide RNA, (iii) at least one transgene, and (iv) shRNA targeting a histone deacetylase (HDAC); wherein following transduction of the one or more cells, the expression and/or activity of the targeted HDAC is decreased and/or depleted; and wherein the generated optimized viral vector has an increased packaging capacity. Viral vectors are disclosed supra.

Disclosed herein is a method of increasing the packaging capacity of a viral vector, the method comprising contacting one or more cells with a viral vector comprising an isolated nucleic acid molecule comprising a nucleic acid sequence encoding (i) a fusion product, (ii) at least one guide RNA, and (iii) and shRNA targeting a histone deacetylase (HDAC); wherein following transduction of the one or more cells, the expression and/or activity of the targeted HDAC is decreased and/or depleted; and wherein the generated optimized viral vector has an increased packaging capacity. Viral vectors are disclosed supra.

In an aspect, the disclosed transduced cells can comprise a mammalian cell line such as human cell line, an equine cell line, a bovine cell line, a porcine cell line, a canine cell line, a feline cell line, or a rodent cells cell line (e.g., rat or mouse). In an aspect, the disclosed transduced cell line can comprise HEK cells (e.g., HEK 293 cells, HEK 293T, HEK 293FT, HEK 293S, HEK 293FT, HEK 293SG, HEK 293SGGD, HEK 293H, HEK 293E, HEK 293A, HEK 293MSR, etc.).

In an aspect, the disclosed method of increasing the packaging capacity can further increase the titer of a generated optimized viral vector. In an aspect, the disclosed method of increasing the packaging capacity can further increase the expression capacity of a generated optimized viral vector. In an aspect, the disclosed method of increasing the packaging capacity can further decrease the immunogenicity and/or toxicity of a generated optimized viral vector. In an aspect, the disclosed method of increasing the packaging capacity can further increase the ability of the generated optimized viral vector to efficiently transduce one or more cells.

In an aspect, the disclosed method of increasing the packaging capacity can further increase the titer, increase the expression capacity, decrease the immunogenicity and/or toxicity, increase the ability of the generated optimized viral vector to efficiently transduce one or more cells, or any combination thereof when compared to cells having normal or wildtype HDAC expression and/or activity.

In an aspect of a disclosed method of increasing the packaging capacity, the disclosed HDAC^{-/-} cells can be HDAC1^{-/-}, HDAC2^{-/-}, HDAC3^{-/-}, HDAC4^{-/-}, HDAC6^{-/-}, HDAC8^{-/-}, or any combination thereof. In an aspect of a disclosed method of increasing the packaging capacity, the disclosed HDAC^{-/-} cells can be HDAC1^{-/-} but still express HDAC2, HDAC3, HDAC4, HDAC6, HDAC8, or any combination thereof. In an aspect of a disclosed method of increasing the packaging capacity, the disclosed HDAC^{-/-} cells can be HDAC2^{-/-} but still express HDAC1, HDAC3, HDAC4, HDAC6, HDAC8, or any combination thereof. In an aspect of a disclosed method of increasing the packaging capacity, the disclosed HDAC^{-/-} cells can be HDAC3^{-/-} but still express HDAC1, HDAC2, HDAC4, HDAC6, HDAC8, or any combination thereof. In an aspect of a disclosed method of increasing the packaging capacity, the disclosed HDAC^{-/-} cells can be HDAC4^{-/-} but still express HDAC1, HDAC2, HDAC3, HDAC6, HDAC8, or any combination thereof. In an aspect of a disclosed method of increasing the packaging capacity, the disclosed HDAC^{-/-} cells can be HDAC6^{-/-} but still express HDAC1, HDAC2, HDAC3, HDAC4, HDAC8, or any combination thereof. In an aspect of a disclosed method of increasing the packaging capacity, the disclosed HDAC^{-/-} cells can be HDAC8^{-/-} but still express HDAC1, HDAC2, HDAC3, HDAC4, HDAC6, or any combination thereof.

In an aspect of a disclosed method of increasing the packaging capacity, the disclosed HDAC^{-/-} cells can comprise a complete decrease and/or depletion in expression and/or activity (e.g., 100% or about 100%). In an aspect, an incomplete decrease and/or depletion in expression and/or activity can comprise at least 50%, at least 55%, at least 60%, at least 65%, at least 70%, at least 75%, at least 80%, at least 85%, at least 90%, at least 95%, or more than 95% decrease and/or depletion when compared to cells having normal or wildtype HDAC expression and/or activity.

In an aspect, the disclosed HDAC^{-/-} cells can be used to generate a ICLV vector or an IDLV vector. In an aspect, the disclosed HDAC^{-/-} cells can be used to generate an AAV vector or rAAV vector. In an aspect, the disclosed HDAC depleted cells can generate a viral vector that is compatible with advanced manufacturing techniques.

In an aspect of a disclosed method of increasing the packaging capacity, the disclosed HDAC^{-/-} cells can generate a viral vector that can efficiently transduce dividing and/or non-dividing cells (e.g., such as, for example, postmitotic neurons of the brain).

In an aspect of a disclosed method of increasing the packaging capacity, the method can further comprise administering a therapeutically effective amount of the generated optimized viral vector to a subject in need thereof. In an aspect, a subject in need thereof can comprise a subject having a genetic defect or disorder. For example, in an aspect, a subject can have a genetic defect or disorder that affects an NDD-related gene.

In an aspect, a disclosed method of increasing the packaging capacity can be a part of all-in-one gene therapy delivery system for in vivo use in a subject (such as, for example, a human being with a genetic disease or disorder or a neurological disease or disorder).

In an aspect, a disclosed method can further comprise determining the HDAC genotype of one or more cells, the packaging capacity of a generated optimized viral vector, the titer of a generated optimized viral vector, the expression capacity of a generated optimized viral vector, the immunogenicity and/or toxicity of a generated optimized viral vector, the ability of generated optimized viral vector to efficiently transduce cells, or any combination thereof.

### 3. Methods of Increasing the Titer of a Viral Vector

Disclosed herein is a method of increasing the titer of a viral vector, the method comprising contacting one or more cells with a disclosed viral vector; wherein following transduction of the one or more cells, the expression and/or activity of the targeted HDAC is decreased and/or depleted; and wherein the titer of the generated optimized viral vector is increased. Viral vectors are disclosed supra.

Disclosed herein is a method of increasing the titer of a viral vector, the method comprising contacting one or more cells with a viral vector comprising an isolated nucleic acid molecule comprising a nucleic acid sequence encoding (i) a Cas endonuclease, (ii) at least one guide RNA, and (iii) at least one transgene; wherein following transduction of the one or more cells, the expression and/or activity of the targeted HDAC is decreased and/or depleted; and wherein the titer of the generated optimized viral vector is increased. Viral vectors are disclosed supra.

Disclosed herein is a method of increasing the titer of a viral vector, the method comprising contacting one or more cells with a viral vector comprising an isolated nucleic acid molecule comprising a nucleic acid sequence encoding (i) a fusion product, and (ii) at least one guide RNA; wherein following transduction of the one or more cells, the expression and/or activity of the targeted HDAC is decreased and/or depleted; and wherein the titer of the generated optimized viral vector is increased. Viral vectors are disclosed supra.

Disclosed herein is a method of increasing the titer of a viral vector, the method comprising contacting one or more cells with a viral vector comprising an isolated nucleic acid molecule comprising a nucleic acid sequence encoding (i) a fusion product, (ii) at least one guide RNA, and (iii) and shRNA targeting a histone deacetylase (HDAC); wherein following transduction of the one or more cells, the expression and/or activity of the targeted HDAC is decreased and/or depleted; and wherein the titer of the generated optimized viral vector is increased. Viral vectors are disclosed supra.

Disclosed herein is a method of increasing the titer of a viral vector, the method comprising contacting one or more cells with a viral vector comprising an isolated nucleic acid molecule comprising a nucleic acid sequence encoding (i) a Cas endonuclease, (ii) at least one guide RNA, (iii) at least one transgene, and (iv) shRNA targeting a histone deacetylase (HDAC); wherein following transduction of the one or more cells, the expression and/or activity of the targeted HDAC is decreased and/or depleted; and wherein the titer of the generated optimized viral vector is increased. Viral vectors are disclosed supra.

In an aspect, the disclosed transduced cells can comprise a mammalian cell line such as human cell line, an equine cell line, a bovine cell line, a porcine cell line, a canine cell line, a feline cell line, or a rodent cells cell line (e.g., rat or mouse). In an aspect, the disclosed transduced cell line can comprise HEK cells (e.g., HEK 293 cells, HEK 293T, HEK 293FT, HEK 293S, HEK 293FT, HEK 293SG, HEK 293SGGD, HEK 293H, HEK 293E, HEK 293A, HEK 293MSR, etc.).

In an aspect, a disclosed method of increasing the titer of a viral vector can further increase the packaging capacity of a generated optimized viral vector. In an aspect, a disclosed method of increasing the titer of a viral vector can further increase the expression capacity of a generated optimized viral vector. In an aspect, a disclosed method of increasing the titer of a viral vector can further decrease the immunogenicity and/or toxicity of a generated optimized viral vector. In an aspect, a disclosed method of increasing the titer of a viral vector can further increase the ability of the generated optimized viral vector to efficiently transduce one or more cells.

In an aspect, a disclosed method of increasing the titer of a viral vector can further increase the packaging capacity, increase the expression capacity, decrease the immunogenicity and/or toxicity, increase the ability of the generated optimized viral vector to efficiently transduce one or more cells, or any combination thereof when compared to cells having normal or wildtype HDAC expression and/or activity.

In an aspect of a disclosed method of increasing the titer of a viral vector, the disclosed HDAC^{-/-} cells can be HDAC1^{-/-}, HDAC2^{-/-}, HDAC3^{-/-}, HDAC4^{-/-}, HDAC6^{-/-}, HDAC8^{-/-}, or any combination thereof. In an aspect of a disclosed method of increasing the titer of a viral vector, the disclosed HDAC^{-/-} cells can be HDAC1^{-/-} but still express HDAC2, HDAC3, HDAC4, HDAC6, HDAC8, or any combination thereof. In an aspect of a disclosed method of increasing the titer of a viral vector, the disclosed HDAC^{-/-} cells can be HDAC2/but still express HDAC1, HDAC3, HDAC4, HDAC6, HDAC8, or any combination thereof. In an aspect of a disclosed method of increasing the titer of a viral vector, the disclosed HDAC^{-/-} cells can be HDAC3^{-/-} but still express HDAC1, HDAC2, HDAC4, HDAC6, HDAC8, or any combination thereof. In an aspect of a disclosed method of increasing the titer of a viral vector, the disclosed HDAC^{-/-} cells can be HDAC4^{-/-} but still express HDAC1, HDAC2, HDAC3, HDAC6, HDAC8, or any combination thereof. In an aspect of a disclosed method of increasing the titer of a viral vector, the disclosed HDAC^{-/-} cells can be HDAC6^{-/-} but still express HDAC1, HDAC2, HDAC3, HDAC4, HDAC8, or any combination thereof. In an aspect of a disclosed method of increasing the titer of a viral vector, the disclosed HDAC^{-/-} cells can be HDAC8^{-/-} but still express HDAC1, HDAC2, HDAC3, HDAC4, HDAC6, or any combination thereof.

In an aspect of a disclosed method of increasing the titer of a viral vector, the disclosed HDAC^{-/-} cells can comprise a complete decrease and/or depletion in expression and/or activity (e.g., 100% or about 100%). In an aspect, an incomplete decrease and/or depletion in expression and/or activity can comprise at least 50%, at least 55%, at least 60%, at least 65%, at least 70%, at least 75%, at least 80%, at least 85%, at least 90%, at least 95%, or more than 95% decrease and/or depletion when compared to cells having normal or wildtype HDAC expression and/or activity.

In an aspect, the disclosed HDAC^{-/-} cells can be used to generate a ICLV vector or an IDLV vector. In an aspect, the disclosed HDAC^{-/-} cells can be used to generate an AAV vector or rAAV vector. In an aspect, the disclosed HDAC depleted cells can generate a viral vector that is compatible with advanced manufacturing techniques.

In an aspect of a disclosed method of increasing the titer of a viral vector, the disclosed HDAC^{-/-} cells can generate a viral vector that can efficiently transduce dividing and/or non-dividing cells (e.g., such as, for example, postmitotic neurons of the brain).

In an aspect of a disclosed method of increasing the titer of a viral vector, the method can further comprise administering a therapeutically effective amount of the generated optimized viral vector to a subject in need thereof. In an aspect, a subject in need thereof can comprise a subject having a genetic defect or disorder. For example, in an aspect, a subject can have a genetic defect or disorder that affects an NDD-related gene.

In an aspect, a disclosed method of increasing the titer of a viral vector can be a part of all-in-one gene therapy delivery system for in vivo use in a subject (such as, for example, a human being with a genetic disease or disorder or a neurological disease or disorder).

In an aspect, a disclosed method can further comprise determining the HDAC genotype of one or more cells, the packaging capacity of a generated optimized viral vector, the titer of a generated optimized viral vector, the expression capacity of a generated optimized viral vector, the immunogenicity and/or toxicity of a generated optimized viral vector, the ability of generated optimized viral vector to efficiently transduce cells, or any combination thereof.

### 4. Methods of Increasing the Expression Capacity of a Viral Vector

Disclosed herein is a method of increasing the expression capacity of a viral vector, the method comprising contacting one or more cells with a disclosed viral vector; wherein following transduction of the one or more cells, the expression and/or activity of the targeted HDAC is decreased and/or depleted; and wherein the generated optimized viral vector has an increased expression capacity is increased. Viral vectors are disclosed supra.

Disclosed herein is a method of increasing the expression capacity of a viral vector, the method comprising contacting one or more cells with a viral vector comprising an isolated nucleic acid molecule comprising a nucleic acid sequence encoding (i) a Cas endonuclease, (ii) at least one guide RNA, and (iii) at least one transgene; wherein following transduction of the one or more cells, the expression and/or activity of the targeted HDAC is decreased and/or depleted; and wherein the generated optimized viral vector has an increased expression capacity is increased. Viral vectors are disclosed supra.

Disclosed herein is a method of increasing the expression capacity of a viral vector, the method comprising contacting one or more cells with a viral vector comprising an isolated nucleic acid molecule comprising a nucleic acid sequence encoding (i) a fusion product, and (ii) at least one guide RNA; wherein following transduction of the one or more cells, the expression and/or activity of the targeted HDAC is decreased and/or depleted; and wherein the generated optimized viral vector has an increased expression capacity is increased. Viral vectors are disclosed supra.

Disclosed herein is a method of increasing the expression capacity of a viral vector, the method comprising contacting one or more cells with a viral vector comprising an isolated nucleic acid molecule comprising a nucleic acid sequence encoding (i) a Cas endonuclease, (ii) at least one guide RNA, (iii) at least one transgene, and (iv) shRNA targeting a histone deacetylase (HDAC); wherein following transduction of the one or more cells, the expression and/or activity of the targeted HDAC is decreased and/or depleted; and wherein the generated optimized viral vector has an increased expression capacity is increased. Viral vectors are disclosed supra.

Disclosed herein is a method of increasing the expression capacity of a viral vector, the method comprising contacting one or more cells with a viral vector comprising an isolated nucleic acid molecule comprising a nucleic acid sequence encoding (i) a fusion product, (ii) at least one guide RNA, and (iii) and shRNA targeting a histone deacetylase (HDAC); wherein following transduction of the one or more cells, the expression and/or activity of the targeted HDAC is decreased and/or depleted; and wherein the generated optimized viral vector has an increased expression capacity is increased. Viral vectors are disclosed supra.

In an aspect, the disclosed transduced cells can comprise a mammalian cell line such as human cell line, an equine cell line, a bovine cell line, a porcine cell line, a canine cell line, a feline cell line, or a rodent cells cell line (e.g., rat or mouse). In an aspect, the disclosed transduced cell line can comprise HEK cells (e.g., HEK 293 cells, HEK 293T, HEK 293FT, HEK 293S, HEK 293FT, HEK 293SG, HEK 293SGGD, HEK 293H, HEK 293E, HEK 293A, HEK 293MSR, etc.).

In an aspect, a disclosed method of increasing the expression capacity of a viral vector can further increase the packaging capacity of a generated optimized viral vector. In an aspect, a disclosed method of increasing the expression capacity of a viral vector can further increase the titer of a generated optimized viral vector. In an aspect, a disclosed method of increasing the expression capacity of a viral vector can further decrease the immunogenicity and/or toxicity of a generated optimized viral vector. In an aspect, a disclosed method of increasing the expression capacity of a viral vector can further increase the ability of the generated optimized viral vector to efficiently transduce one or more cells.

In an aspect, a disclosed method of increasing the expression capacity of a viral vector can further increase the packaging capacity, increase the titer decrease the immunogenicity and/or toxicity, increase the ability of the generated optimized viral vector to efficiently transduce one or more cells, or any combination thereof when compared to cells having normal or wildtype HDAC expression and/or activity.

In an aspect of a disclosed method of increasing the expression capacity of a viral vector, the disclosed HDAC^{-/-} cells can be HDAC1^{-/-}, HDAC2^{-/-}, HDAC3^{-/-}, HDAC4^{-/-}, HDAC6^{-/-}, HDAC8^{-/-}, or any combination thereof. In an aspect of a disclosed method of increasing the expression capacity of a viral vector, the disclosed HDAC^{-/-} cells can be HDAC1^{-/-} but still express HDAC2, HDAC3, HDAC4, HDAC6, HDAC8, or any combination thereof. In an aspect of a disclosed method of increasing the expression capacity of a viral vector, the disclosed HDAC^{-/-} cells can be HDAC2^{-/-} but still express HDAC1, HDAC3, HDAC4, HDAC6, HDAC8, or any combination thereof. In an aspect of a disclosed method of increasing the expression capacity of a viral vector, the disclosed HDAC^{-/-} cells can be HDAC3^{-/-} but still express HDAC1, HDAC2, HDAC4, HDAC6, HDAC8, or any combination thereof. In an aspect of a disclosed method of increasing the expression capacity of a viral vector, the disclosed HDAC^{-/-} cells can be HDAC4^{-/-} but still express HDAC1, HDAC2, HDAC3, HDAC6, HDAC8, or any combination thereof. In an aspect of a disclosed method of increasing the expression capacity of a viral vector, the disclosed HDAC^{-/-} cells can be HDAC6^{-/-} but still express HDAC1, HDAC2, HDAC3, HDAC4, HDAC8, or any combination thereof. In an aspect of a disclosed method of increasing the expression capacity of a viral vector, the disclosed HDAC^{-/-} cells can be HDAC8^{-/-} but still express HDAC1, HDAC2, HDAC3, HDAC4, HDAC6, or any combination thereof.

In an aspect of a disclosed method of increasing the expression capacity of a viral vector, the disclosed HDAC^{-/-} cells can comprise a complete decrease and/or depletion in expression and/or activity (e.g., 100% or about 100%). In an aspect, an incomplete decrease and/or depletion in expression and/or activity can comprise at least 50%, at least 55%, at least 60%, at least 65%, at least 70%, at least 75%, at least 80%, at least 85%, at least 90%, at least 95%, or more than 95% decrease and/or depletion when compared to cells having normal or wildtype HDAC expression and/or activity.

In an aspect, the disclosed HDAC^{-/-} cells can be used to generate a ICLV vector or an IDLV vector. In an aspect, the disclosed HDAC^{-/-} cells can be used to generate an AAV vector or rAAV vector. In an aspect, the disclosed HDAC depleted cells can generate a viral vector that is compatible with advanced manufacturing techniques.

In an aspect of a disclosed method of increasing the expression capacity of a viral vector, the disclosed HDAC^{-/-} cells can generate a viral vector that can efficiently transduce dividing and/or non-dividing cells (e.g., such as, for example, postmitotic neurons of the brain).

In an aspect of a disclosed method of increasing the expression capacity of a viral vector, the method can further comprise administering a therapeutically effective amount of the generated optimized viral vector to a subject in need thereof. In an aspect, a subject in need thereof can comprise a subject having a genetic defect or disorder. For example, in an aspect, a subject can have a genetic defect or disorder that affects an NDD-related gene.

In an aspect, a disclosed method of increasing the expression capacity of a viral vector can be a part of all-in-one gene therapy delivery system for in vivo use in a subject (such as, for example, a human being with a genetic disease or disorder or a neurological disease or disorder).

In an aspect, a disclosed method can further comprise determining the HDAC genotype of one or more cells, the packaging capacity of a generated optimized viral vector, the titer of a generated optimized viral vector, the expression capacity of a generated optimized viral vector, the immunogenicity and/or toxicity of a generated optimized viral vector, the ability of generated optimized viral vector to efficiently transduce cells, or any combination thereof.

5. Methods of Decreasing the Immunogenicity and/or Toxicity of a Viral Vector

Disclosed herein is a method of decreasing the immunogenicity and/or toxicity of a viral vector, the method comprising contacting one or more cells with a disclosed viral vector; wherein following transduction of the one or more cells, the expression and/or activity of the targeted HDAC is decreased and/or depleted; and wherein the generated optimized viral vectors have decreased immunogenicity and/or toxicity. Viral vectors are disclosed supra.

Disclosed herein is a method of decreasing the immunogenicity and/or toxicity of a viral vector, the method comprising contacting one or more cells with a viral vector comprising an isolated nucleic acid molecule comprising a nucleic acid sequence encoding (i) a Cas endonuclease, (ii) at least one guide RNA, and (iii) at least one transgene; wherein following transduction of the one or more cells, the expression and/or activity of the targeted HDAC is decreased and/or depleted; and wherein the generated optimized viral vectors have decreased immunogenicity and/or toxicity. Viral vectors are disclosed supra.

Disclosed herein is a method of decreasing the immunogenicity and/or toxicity of a viral vector, the method comprising contacting one or more cells with a viral vector comprising an isolated nucleic acid molecule comprising a nucleic acid sequence encoding (i) a fusion product, and (ii) at least one guide RNA; wherein following transduction of the one or more cells, the expression and/or activity of the targeted HDAC is decreased and/or depleted; and wherein the generated optimized viral vectors have decreased immunogenicity and/or toxicity. Viral vectors are disclosed supra.

Disclosed herein is a method of decreasing the immunogenicity and/or toxicity of a viral vector, the method comprising contacting one or more cells with a viral vector comprising an isolated nucleic acid molecule comprising a nucleic acid sequence encoding (i) a Cas endonuclease, (ii) at least one guide RNA, (iii) at least one transgene, and (iv) shRNA targeting a histone deacetylase (HDAC); wherein following transduction of the one or more cells, the expression and/or activity of the targeted HDAC is decreased and/or depleted; and wherein the generated optimized viral vectors have decreased immunogenicity and/or toxicity. Viral vectors are disclosed supra.

Disclosed herein is a method of decreasing the immunogenicity and/or toxicity of a viral vector, the method comprising contacting one or more cells with a viral vector comprising an isolated nucleic acid molecule comprising a nucleic acid sequence encoding (i) a fusion product, (ii) at least one guide RNA, and (iii) and shRNA targeting a histone deacetylase (HDAC); wherein following transduction of the one or more cells, the expression and/or activity of the targeted HDAC is decreased and/or depleted; and wherein the generated optimized viral vectors have decreased immunogenicity and/or toxicity. Viral vectors are disclosed supra.

In an aspect, the disclosed transduced cells can comprise a mammalian cell line such as human cell line, an equine cell line, a bovine cell line, a porcine cell line, a canine cell line, a feline cell line, or a rodent cells cell line (e.g., rat or mouse). In an aspect, the disclosed transduced cell line can comprise HEK cells (e.g., HEK 293 cells, HEK 293T, HEK 293FT, HEK 293S, HEK 293FT, HEK 293SG, HEK 293SGGD, HEK 293H, HEK 293E, HEK 293A, HEK 293MSR, etc.).

In an aspect, a disclosed method of decreasing the immunogenicity and/or toxicity of a viral vector can further increase the packaging capacity of a generated optimized viral vector. In an aspect, a disclosed method of decreasing the immunogenicity and/or toxicity of a viral vector can further increase the titer of a generated optimized viral vector. In an aspect, a disclosed method of decreasing the immunogenicity and/or toxicity of a viral vector can further increase the expression capacity of a generated optimized viral vector. In an aspect, a disclosed method of decreasing the immunogenicity and/or toxicity can further increase the ability of the generated optimized viral vector to efficiently transduce one or more cells.

In an aspect, a disclosed method of decreasing the immunogenicity and/or toxicity of a viral vector can further increase the packaging capacity of a generated optimized viral vector, increase the titer of a generated optimized viral vector, increase the expression capacity of a generated optimized viral vector, increase the ability of a generated optimized viral vector to efficiently transduce one or more cells, or any combination thereof when compared to cells having normal or wildtype HDAC expression and/or activity.

In an aspect of a disclosed method of decreasing the immunogenicity and/or toxicity of a viral vector, the disclosed HDAC^{-/-} cells can be HDAC1^{-/-}, HDAC2^{-/-}, HDAC3^{-/-}, HDAC4^{-/-}, HDAC6^{-/-}, HDAC8^{-/-}, or any combination thereof. In an aspect of a disclosed method of decreasing the immunogenicity and/or toxicity of a viral vector, the disclosed HDAC^{-/-} cells can be HDAC1^{-/-} but still express HDAC2, HDAC3, HDAC4, HDAC6, HDAC8, or any combination thereof. In an aspect of a disclosed method of decreasing the immunogenicity and/or toxicity of a viral vector, the disclosed HDAC^{-/-} cells can be HDAC2^{-/-} but still express HDAC1, HDAC3, HDAC4, HDAC6, HDAC8, or any combination thereof. In an aspect of a disclosed method of decreasing the immunogenicity and/or toxicity of a viral vector, the disclosed HDAC^{-/-} cells can be HDAC3^{-/-} but still express HDAC1, HDAC2, HDAC4, HDAC6, HDAC8, or any combination thereof. In an aspect of a disclosed method of decreasing the immunogenicity and/or toxicity of a viral vector, the disclosed HDAC^{-/-} cells can be HDAC4^{-/-} but still express HDAC1, HDAC2, HDAC3, HDAC6, HDAC8, or any combination thereof. In an aspect of a disclosed method of decreasing the immunogenicity and/or toxicity of a viral vector, the disclosed HDAC^{-/-} cells can be HDAC6^{-/-} but still express HDAC1, HDAC2, HDAC3, HDAC4, HDAC8, or any combination thereof. In an aspect of a disclosed method of decreasing the immunogenicity and/or toxicity of a viral vector, the disclosed HDAC^{-/-} cells can be HDAC8^{-/-} but still express HDAC1, HDAC2, HDAC3, HDAC4, HDAC6, or any combination thereof.

In an aspect of a disclosed method of decreasing the immunogenicity and/or toxicity of a viral vector, the disclosed HDAC^{-/-} cells can comprise a complete decrease and/or depletion in expression and/or activity (e.g., 100% or about 100%). In an aspect, an incomplete decrease and/or depletion in expression and/or activity can comprise at least 50%, at least 55%, at least 60%, at least 65%, at least 70%, at least 75%, at least 80%, at least 85%, at least 90%, at least 95%, or more than 95% decrease and/or depletion when compared to cells having normal or wildtype HDAC expression and/or activity.

In an aspect, the disclosed HDAC^{-/-} cells can be used to generate a ICLV vector or an IDLV vector. In an aspect, the disclosed HDAC^{-/-} cells can be used to generate an AAV vector or rAAV vector. In an aspect, the disclosed HDAC depleted cells can generate a viral vector that is compatible with advanced manufacturing techniques.

In an aspect of a disclosed method of decreasing the immunogenicity and/or toxicity of a viral vector, the disclosed HDAC^{-/-} cells can generate a viral vector that can efficiently transduce dividing and/or non-dividing cells (e.g., such as, for example, postmitotic neurons of the brain).

In an aspect of a disclosed method of decreasing the immunogenicity and/or toxicity of a viral vector, the method can further comprise administering a therapeutically effective amount of the generated optimized viral vector to a subject in need thereof. In an aspect, a subject in need thereof can comprise a subject having a genetic defect or disorder. For example, in an aspect, a subject can have a genetic defect or disorder that affects an NDD-related gene.

In an aspect, a disclosed method of decreasing the immunogenicity and/or toxicity of a viral vector can be a part of all-in-one gene therapy delivery system for in vivo use in a subject (such as, for example, a human being with a genetic disease or disorder or a neurological disease or disorder).

In an aspect, a disclosed method can further comprise determining the HDAC genotype of one or more cells, the packaging capacity of a generated optimized viral vector, the titer of a generated optimized viral vector, the expression capacity of a generated optimized viral vector, the immunogenicity and/or toxicity of a generated optimized viral vector, the ability of generated optimized viral vector to efficiently transduce cells, or any combination thereof.

### 6. Methods of Increasing the Ability of a Viral Vector to Efficiently Transduce Cells

Disclosed herein is a method of increasing the ability of a viral vector to efficiently transduce one or more cells, the method comprising contacting one or more cells with a disclosed viral vector; wherein following transduction of the one or more cells, the expression and/or activity of the targeted HDAC is decreased and/or depleted; and wherein the generated optimized viral vectors have an increased ability to efficiently transduce cells. Viral vectors are disclosed supra.

Disclosed herein is a method of increasing the ability of a viral vector to efficiently transduce one or more cells, the method comprising contacting one or more cells with a viral vector comprising an isolated nucleic acid molecule comprising a nucleic acid sequence encoding (i) a Cas endonuclease, (ii) at least one guide RNA, and (iii) at least one transgene; wherein following transduction of the one or more cells, the expression and/or activity of the targeted HDAC is decreased and/or depleted; and wherein the generated optimized viral vectors have an increased ability to efficiently transduce cells. Viral vectors are disclosed supra.

Disclosed herein is a method of increasing the ability of a viral vector to efficiently transduce one or more cells, the method comprising contacting one or more cells with a viral vector comprising an isolated nucleic acid molecule comprising a nucleic acid sequence encoding (i) a fusion product, and (ii) at least one guide RNA; wherein following transduction of the one or more cells, the expression and/or activity of the targeted HDAC is decreased and/or depleted; and wherein the generated optimized viral vectors have an increased ability to efficiently transduce cells. Viral vectors are disclosed supra.

Disclosed herein is a method of increasing the ability of a viral vector to efficiently transduce one or more cells, the method comprising contacting one or more cells with a viral vector comprising an isolated nucleic acid molecule comprising a nucleic acid sequence encoding (i) a Cas endonuclease, (ii) at least one guide RNA, (iii) at least one transgene, and (iv) shRNA targeting a histone deacetylase (HDAC); wherein following transduction of the one or more cells, the expression and/or activity of the targeted HDAC is decreased and/or depleted; and wherein the generated optimized viral vectors have an increased ability to efficiently transduce cells. Viral vectors are disclosed supra.

Disclosed herein is a method of increasing the ability of a viral vector to efficiently transduce one or more cells, the method comprising contacting one or more cells with a viral vector comprising an isolated nucleic acid molecule comprising a nucleic acid sequence encoding (i) a fusion product, (ii) at least one guide RNA, and (iii) and shRNA targeting a histone deacetylase (HDAC); wherein following transduction of the one or more cells, the expression and/or activity of the targeted HDAC is decreased and/or depleted; and wherein the generated optimized viral vectors have an increased ability to efficiently transduce cells. Viral vectors are disclosed supra.

In an aspect, the disclosed transduced cells can comprise a mammalian cell line such as human cell line, an equine cell line, a bovine cell line, a porcine cell line, a canine cell line, a feline cell line, or a rodent cells cell line (e.g., rat or mouse). In an aspect, the disclosed transduced cell line can comprise HEK cells (e.g., HEK 293 cells, HEK 293T, HEK 293FT, HEK 293S, HEK 293FT, HEK 293SG, HEK 293SGGD, HEK 293H, HEK 293E, HEK 293A, HEK 293MSR, etc.).

In an aspect, a disclosed method of increasing the ability of a viral vector to efficiently transduce one or more cells can further increase the packaging capacity of a generated optimized viral vector. In an aspect, a disclosed method of increasing the ability of a viral vector to efficiently transduce one or more cells can further increase the titer of a generated optimized viral vector. In an aspect, a disclosed method of increasing the ability of a viral vector to efficiently transduce one or more cells can further increase the expression capacity of a generated optimized viral vector. In an aspect, a disclosed method of increasing the ability of a viral vector to efficiently transduce one or more cells can further decrease the immunogenicity and/or toxicity of a generated optimized viral vector.

In an aspect, a disclosed method of increasing the ability of a viral vector to efficiently transduce one or more cells can further increase the packaging capacity of a generated optimized viral vector, increase the titer of a generated optimized viral vector, increase the expression capacity of a generated optimized viral vector, decrease the immunogenicity and/or toxicity of a generated optimized viral vector, or any combination thereof when compared to cells having normal or wildtype HDAC expression and/or activity.

In an aspect of a disclosed method of increasing the ability of a viral vector to efficiently transduce one or more cells, the disclosed HDAC^{-/-} cells can be HDAC1^{-/-}, HDAC2^{-/-}, HDAC3^{-/-}, HDAC4^{-/-}, HDAC6^{-/-}, HDAC8^{-/-}, or any combination thereof. In an aspect of a disclosed method of increasing the ability of a viral vector to efficiently transduce one or more cells, the disclosed HDAC^{-/-} cells can be HDAC1^{-/-} but still express HDAC2, HDAC3, HDAC4, HDAC6, HDAC8, or any combination thereof. In an aspect of a disclosed method of increasing the ability of a viral vector to efficiently transduce one or more cells, the disclosed HDAC^{-/-} cells can be HDAC2^{-/-} but still express HDAC1, HDAC3, HDAC4, HDAC6, HDAC8, or any combination thereof. In an aspect of a disclosed method of increasing the ability of a viral vector to efficiently transduce one or more cells, the disclosed HDAC^{-/-} cells can be HDAC3^{-/-} but still express HDAC1, HDAC2, HDAC4, HDAC6, HDAC8, or any combination thereof. In an aspect of a disclosed method of increasing the ability of a viral vector to efficiently transduce one or more cells, the disclosed HDAC^{-/-} cells can be HDAC4^{-/-} but still express HDAC1, HDAC2, HDAC3, HDAC6, HDAC8, or any combination thereof. In an aspect of a disclosed method of increasing the ability of a viral vector to efficiently transduce one or more cells, the disclosed HDAC^{-/-} cells can be HDAC6^{-/-} but still express HDAC1, HDAC2, HDAC3, HDAC4, HDAC8, or any combination thereof. In an aspect of a disclosed method of increasing the ability of a viral vector to efficiently transduce one or more cells, the disclosed HDAC^{-/-} cells can be HDAC8^{-/-} but still express HDAC1, HDAC2, HDAC3, HDAC4, HDAC6, or any combination thereof.

In an aspect of a disclosed method of increasing the ability of a viral vector to efficiently transduce one or more cells, the disclosed HDAC^{-/-} cells can comprise a complete decrease and/or depletion in expression and/or activity (e.g., 100% or about 100%). In an aspect, an incomplete decrease and/or depletion in expression and/or activity can comprise at least 50%, at least 55%, at least 60%, at least 65%, at least 70%, at least 75%, at least 80%, at least 85%, at least 90%, at least 95%, or more than 95% decrease and/or depletion when compared to cells having normal or wildtype HDAC expression and/or activity.

In an aspect, the disclosed HDAC^{-/-} cells can be used to generate a ICLV vector or an IDLV vector. In an aspect, the disclosed HDAC^{-/-} cells can be used to generate an AAV vector or rAAV vector. In an aspect, the disclosed HDAC depleted cells can generate a viral vector that is compatible with advanced manufacturing techniques. In an aspect of a disclosed method of increasing the ability of a viral vector to efficiently transduce one or more cells, the disclosed HDAC^{-/-} cells can generate a viral vector that can efficiently transduce dividing and/or non-dividing cells (e.g., such as, for example, postmitotic neurons of the brain).

In an aspect of a disclosed method of increasing the ability of a viral vector to efficiently transduce one or more cells, the method can further comprise administering a therapeutically effective amount of the generated optimized viral vector to a subject in need thereof. In an aspect, a subject in need thereof can comprise a subject having a genetic defect or disorder. For example, in an aspect, a subject can have a genetic defect or disorder that affects an NDD-related gene.

In an aspect, a disclosed method increasing the ability of a viral vector to efficiently transduce one or more cells can be a part of all-in-one gene therapy delivery system for in vivo use in a subject (such as, for example, a human being with a genetic disease or disorder or a neurological disease or disorder).

### D. Miscellaneous

Disclosed herein is a composition comprising Sp1 and NF-kB contained vectors and provided in SEQ ID NO:01. Disclosed herein is a composition comprising Sp1 and NF-kB contained vectors harboring gRNA to GFP and provided in SEQ ID NO:02. Disclosed herein is a composition comprising gRNA to GFP and provided in SEQ ID NO:05 or SEQ ID NO:06.

Disclosed herein is a composition comprising HDAC2 ORF and provided in SEQ ID NO:08. Disclosed herein is a composition comprising HDAC3 ORF and provided in SEQ ID NO:09. Disclosed herein is a composition comprising HDAC4 ORF and provided SEQ ID NO:10. Disclosed herein is a composition comprising HDAC6 ORF and provided in SEQ ID NO:11. Disclosed herein is a composition comprising HDAC8 ORF and provided in SEQ ID NO:12.

Disclosed herein is a composition comprising HDAC1 sequence that has been targeted with shRNA clone TRCN0000195467 and provided in SEQ ID NO:19. Disclosed herein is a composition comprising an HDAC2 sequence that has been targeted with shRNA clone TRCN0000004819 and provided in SEQ ID NO:20. Disclosed herein is a composition comprising an HDAC3 sequence that has been targeted with shRNA clone TRCN0000194993 and provided in SEQ ID NO:21. Disclosed herein is a composition comprising an HDAC4 sequence that has been targeted with shRNA clone TRCN0000004832 and provided in SEQ ID NO:22. Disclosed herein is a composition comprising an HDAC6 sequence that has been targeted with shRNA clone TRCN0000004839 and provided in SEQ ID NO:23. Disclosed herein is a composition comprising an HDAC8 sequence that has been targeted with shRNA clone TRCN0000004839 and provided in SEQ ID NO:24.

Disclosed herein is a composition comprising pLK0.1 viral vector backbone carrying scramble shRNA that targets no mammalian genes and provided in SEQ ID NO:25. Disclosed herein is a composition comprising a pLK0.1 viral vector backbone carrying HDAC1 shRNA and provided in SEQ ID NO:26. Disclosed herein is a composition comprising a pLK0.1 viral vector backbone carrying HDAC2 shRNA and provided in SEQ ID NO:27. Disclosed herein is a composition comprising a pLK0.1 viral vector backbone carrying HDAC3 shRNA and provided in SEQ ID NO:28. Disclosed herein is a composition comprising a pLK0.1 viral vector backbone carrying HDAC4 shRNA and provided in SEQ ID NO:29. Disclosed herein is a composition comprising a pLK0.1 viral vector backbone carrying HDAC6 shRNA and provided in SEQ ID NO:30. Disclosed herein is a composition comprising a pLK0.1 viral vector backbone carrying HDAC8 shRNA and provided in SEQ ID NO:31.

| SEQ ID NO: | Description |
|---|---|
| SEQ ID NO: 01 | LV Vector comprising Sp1 and NF-kB transcription factor |
| | recognition sites |
| SEQ ID NO: 02 | LV Vector comprising Sp1 and NF-kB transcription factor |
| | recognition sites and shRNA targeting GFP |
| SEQ ID NO: 03 | Green Fluorescent Protein |
| SEQ ID NO: 04 | Green Fluorescent Protein |
| SEQ ID NO: 05 | gRNA targeted to GFP |
| SEQ ID NO: 06 | gRNA targeted to GFP |
| SEQ ID NO: 07 | ORF of Histone Deacetylase 1 (HDAC1) |
| SEQ ID NO: 08 | ORF of Histone Deacetylase 2 (HDAC2) |
| SEQ ID NO: 09 | ORF of Histone Deacetylase 3 (HDAC3) |
| SEQ ID NO: 10 | ORF of Histone Deacetylase 4 (HDAC4) |
| SEQ ID NO: 11 | ORF for Histone Deacetylase 6 (HDAC6) |
| SEQ ID NO: 12 | ORF for Histone Deacetylase 8 (HDAC8) |
| SEQ ID NO: 13 | Histone Deacetylase 1 (HDAC1) |
| SEQ ID NO: 14 | Histone Deacetylase 2 (HDAC2) |
| SEQ ID NO: 15 | Histone Deacetylase 3 (HDAC3) |
| SEQ ID NO: 16 | Histone Deacetylase 4 (HDAC4) |
| SEQ ID NO: 17 | Histone Deacetylase 6 (HDAC6) |
| SEQ ID NO: 18 | Histone Deacetylase 8 (HDAC8) |
| SEQ ID NO: 19 | shRNA targeting HDAC1 |
| SEQ ID NO: 20 | shRNA targeting HDAC2 |
| SEQ ID NO: 21 | shRNA targeting HDAC3 |
| SEQ ID NO: 22 | shRNA targeting HDAC4 |
| SEQ ID NO: 23 | shRNA targeting HDAC6 |
| SEQ ID NO: 24 | shRNA targeting HDAC8 |
| SEQ ID NO: 25 | SHC002 - TRC1.5 pLK0.1-puro Non-Mammalian shRNA Control |
| | Plasmid DNA carrying scramble shRNA that targets no mammalian |
| | genes |
| SEQ ID NO: 26 | pLK0.1 vector backbone carrying shRNA targeting HDAC1 |
| SEQ ID NO: 27 | pLK0.1 vector backbone carrying shRNA targeting HDAC2 |
| SEQ ID NO: 28 | pLK0.1 vector backbone carrying shRNA targeting HDAC3 |
| SEQ ID NO: 29 | pLK0.1 vector backbone carrying shRNA targeting HDAC4 |
| SEQ ID NO: 30 | pLK0.1 vector backbone carrying shRNA targeting HDAC6 |
| SEQ ID NO: 31 | pLK0.1 vector backbone carrying shRNA targeting HDAC8 |
| SEQ ID NO: 32 | Sequence targeted by sgRNA-GFP in cells transduced with ICLV |
| SEQ ID NO: 33 | Deletion identified by sgRNA-GFP in cells transduced with ICLV |
| SEQ ID NO: 34 | Deletion identified by sgRNA-GFP in cells transduced with ICLV |
| SEQ ID NO: 35 | Insertion identified by sgRNA-GFP in cells transduced with ICLV |
| SEQ ID NO: 36 | Deletion identified by sgRNA-GFP in cells transduced with ICLV |
| SEQ ID NO: 37 | Insertion identified by sgRNA-GFP in cells transduced with ICLV |
| SEQ ID NO: 38 | Deletion identified by sgRNA-GFP in cells transduced with ICLV |
| SEQ ID NO: 39 | Deletion identified by sgRNA-GFP in cells transduced with ICLV |
| SEQ ID NO: 40 | Deletion identified by sgRNA-GFP in cells transduced with ICLV |
| SEQ ID NO: 41 | Substitution identified by sgRNA-GFP in cells transduced with ICLV |
| SEQ ID NO: 42 | Sequence Targeted by sgRNA-GFP in cells transduced with IDLV |
| SEQ ID NO: 43 | Insertion identified by sgRNA-GFP in cells transduced with IDLV |
| SEQ ID NO: 44 | Insertion identified by sgRNA-GFP in cells transduced with IDLV |
| SEQ ID NO: 45 | Deletion identified by sgRNA-GFP in cells transduced with IDLV |
| SEQ ID NO: 46 | Insertion identified by sgRNA-GFP in cells transduced with IDLV |
| SEQ ID NO: 47 | Deletion identified by sgRNA-GFP in cells transduced with IDLV |
| SEQ ID NO: 48 | Deletion identified by sgRNA-GFP in cells transduced with IDLV |
| SEQ ID NO: 49 | Deletion identified by sgRNA-GFP in cells transduced with IDLV |
| SEQ ID NO: 50 | Deletion identified by sgRNA-GFP in cells transduced with IDLV |
| SEQ ID NO: 51 | Deletion identified by sgRNA-GFP in cells transduced with IDLV |
| SEQ ID NO: 52 | Cas9 |
| SEQ ID NO: 53 | Cas9 |
| SEQ ID NO: 54 | Dcas9 |
| SEQ ID NO: 55 | Vrer |
| SEQ ID NO: 56 | MeCP2 Transcription Repression Domain (TRD) |
| SEQ ID NO: 57 | MeCP Transcription Repression Domain (TRD) |
| SEQ ID NO: 58 | Krab-MeCP2 Repressor |
| SEQ ID NO: 59 | Krab-MeCP2 Repressor (ORF) |
| SEQ ID NO: 60 | DNMT3a |
| SEQ ID NO: 61 | DNMT3a |
| SEQ ID NO: 62 | dCas9-DNM 3a Fusion Protein |
| SEQ ID NO: 63 | dCas9-DNMT3a Fusion Protein |
| SEQ ID NO: 64 | VRER-DNMT3a Fusion Protein |
| SEQ ID NO: 65 | VRER-DNMT3a Fusion Protein |

### VII. EXAMPLES

The CRISPR/Cas9 systems have revolutionized the field of genome editing by providing unprecedented control over gene sequences and gene expression in many species, including humans. Lentiviral vectors (LVs) are one of the primary delivery platforms for the CRISPR/Cas9 system due to their ability to accommodate large DNA payloads and sustain robust expression in a wide range of dividing and non-dividing cells. Since the first publication demonstrating the efficient transduction of lentiviral vectors into post-mitotic neurons in vivo (Naldini L, et al. (1996) Science. 272(5259):263-267), thousands of studies have probed the use of HIV-based vectors for gene delivery into the CNS and other tissues and organs. The vector has been successfully used in many clinical trials including Parkinson's disease, Alzheimer's disease, Wiskott-Aldrich syndrome, X-linked adrenoleukodystrophy, Metachromatic leukodystrophy and others (reviewed in Kantor B, et al. (2014) Adv Genet, 87:71-124). Lentivirus was used to target epigenetically the SNCA gene (Kantor B, et al. (2018) Mol Ther. 26(11):2638-2649).

Notwithstanding the success, a broad use of integrase-competent vectors (ICLVs) can be hampered by a relatively high risk of insertional mutagenesis (Ortinski P I, et al. (2017) Mol Ther Methods Clin Dev. 5:153-164). Furthermore, long-term expression of LV-delivering Cas9/gRNA may lead to undesirable off-target effects characterized by non-specific RNA-DNA interactions and off-target DNA perturbations (Ortinski P I, et al. (2017) Mol Ther Methods Clin Dev. 5:153-164). The obvious choice to avert these shortcomings would be the use of adeno-associated vector (AAV). In fact, AAV vectors hat have become a widely adopted platform for delivery of CRISPR/Cas9 components in recent years. However, the large size of the CRISPR/Cas9 transgenes imposes a significant burden on the packaging capacity of AAVs (reviewed in Rittiner J E, et al. (2020) Front Mol Neurosci. 13:148). In contrast with AAV vectors, integrase-deficient lentiviral vectors (IDLVs) own large packaging capacity (up-to 11 kbs), as such are more suitable for CRISPR/Cas9 delivery. In addition, IDLVs demonstrate a broad range of cellular and tissue tropism and shown minimal cytotoxicity and immunogenicity. Importantly, they retain very weak integration capability and as such, are safer than their integrase-competent counterparts.

However, high vector copy numbers of AAV can cause severe toxicity in non-human primates, which underscores the urgent need for improving the manufacturing technology of viral vectors. In fact, serious safety concerns directly related to the manufacturing practice recently set back the IGNITE study, which is a phase I/II clinical trial targeted Duchenne muscular dystrophy (DMD). The sponsoring company (which led this clinical trial) announced that two patients injected with SGT-001, a novel AAV-based system experienced a serious adverse event. In a different clinical trial using AAV in children with X-linked myotubular myopathy (XMTM), the sponsoring company reported the deaths of three boys received high-dose of the experimental vectors. Further still, the sponsoring company suspended clinical trials evaluating Lenti-Globin (a gene therapy for sickle cell disease) after a patient who was treated more than five years ago developed acute myeloid leukemia (AML). The sponsoring company recently announced the termination of development of BIIB089, an investigational SMA1 gene therapy, citing similar toxicity concerns, which could be related to the viral expression cassette that was utilized. In all those studies, serious concern has been expressed over the safety, efficiency, and scalability of the viral vector manufacturing process.

Viral-mediated gene transfer represents a method-of-choice for the delivery of therapeutic cargo to the nervous system. Crucially, it usually requires only a single injection, whether that be a gene replacement strategy for an inherited disorder or the delivery of a genome- or epigenome-modifying construct for treatment of CNS diseases and disorders. It is thus understandable that considerable effort has been put towards the development of improved vector systems for gene transfer into different tissues and organs including the CNS. Different viral vectors are of course tailored to their specific applications, but they generally should share several key properties.

The specific Examples provided below demonstrate that the ideal viral vector incorporates a high-packaging capacity (which is especially valuable for the delivery of oversized CRISPR/Cas tools), efficient gene transfer paired with robust and sustained expression, improved safety including lack of oncogenicity, toxicity and pathogenicity, and scalable manufacturing for clinical applications.

### Example 1

### Construction of Lentiviral Vector Cassette Plasmids

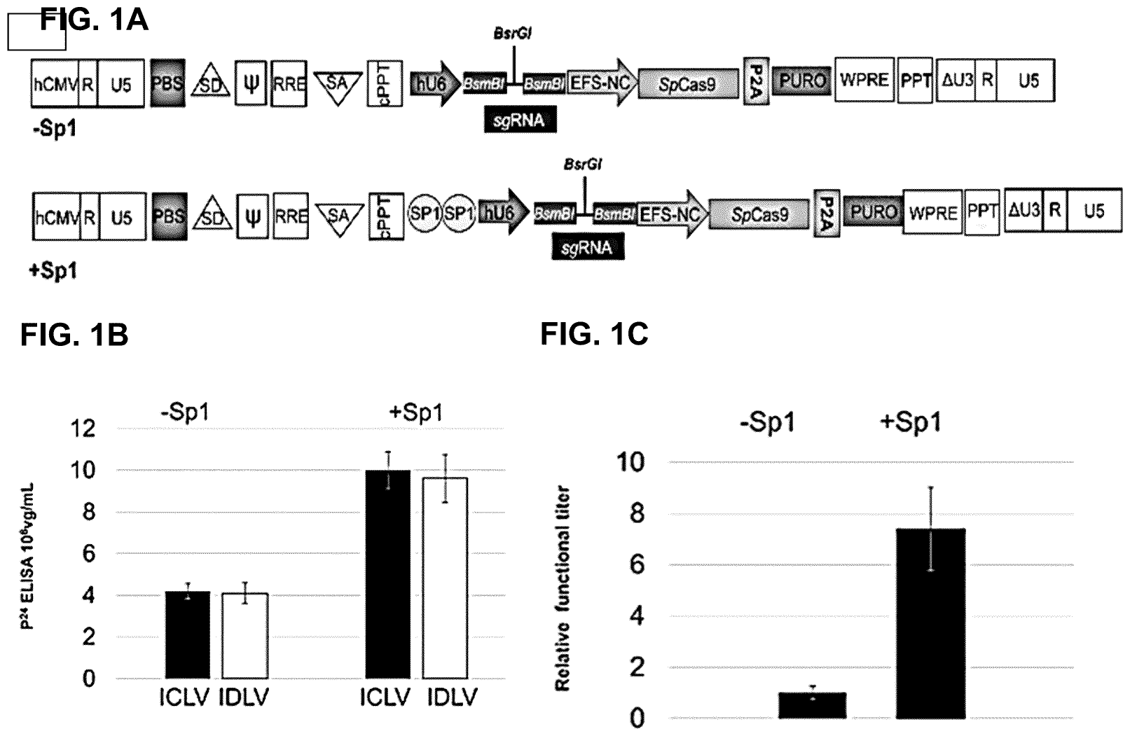

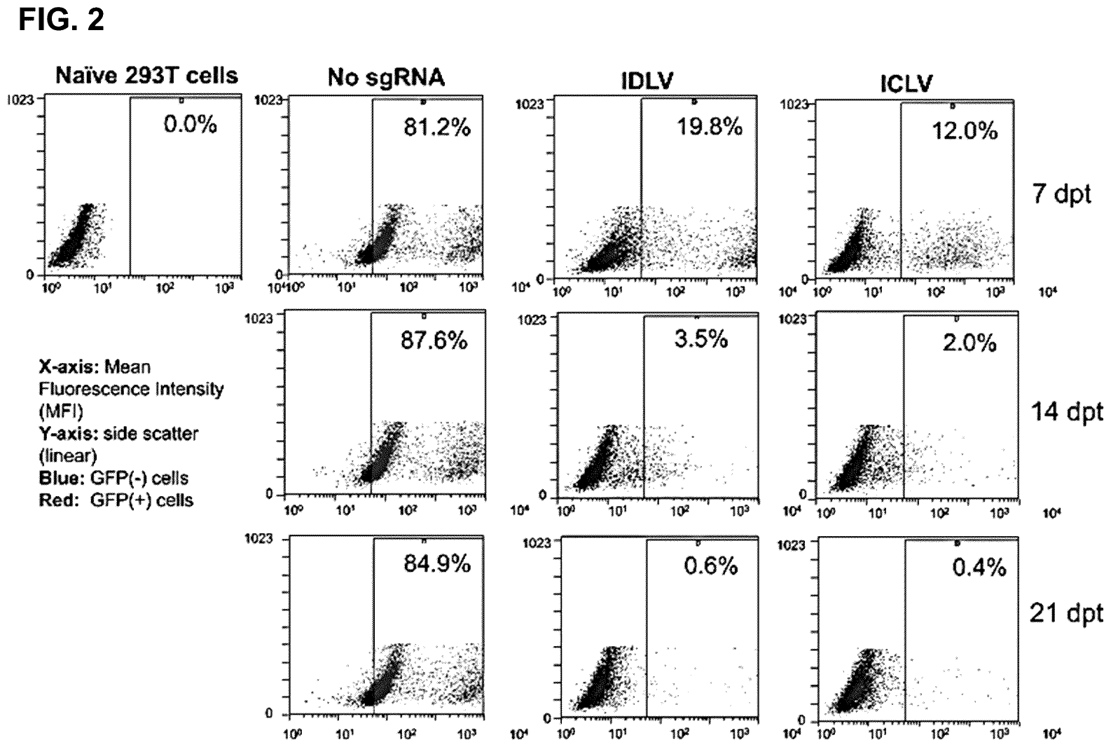

As shown in FIG. 1A, two lentiviral vector cassette plasmids were constructed. The version on the top lacks an Sp1 binding site while the version on the bottom contains two Sp1 binding sites. Other regulatory elements include primer binding site (PBS), splice donor (SD) and splice acceptor (SA), central polypurine tract (cPPT) and polypurine tract (PPT), Rev Response element (RRE), Woodchuck Hepatitis Virus Posttranscriptional Regulatory Element (WPRE), and the retroviral vector packaging element (the psi (W) signal). A human Cytomegalovirus (hCMV) promoter, a core-elongation factor 1a promoter (EFS), and a human U6 promoter are also included. The self-inactivated vector (SIN) cassette plasmid carries a deletion (−18 bps to −418 bps) in the U3 region of 3′-LTR (DU3). A polylinker site contains a pair of BsmBI sites and a unique BsrGI site used for cloning of sgRNA and for its verification, respectively.

To this end, FIG. 1B presents production titers of integrating (ICLV) and non-integrating (IDLV) viral particles+/−Sp1 sites. The results were recorded in copy number per milliliter, equating 1 ng of p24gag to 1×10⁴ particles. FIG. 1C shows the overall ICLV production titer determined by counting puromycin-resistant colonies. The results are shown as the ratio between number of colonies obtained from the vector with and without Sp1. The bar graph data represents mean±SD from triplicate experiments.

### Example 2

### Efficiency of CRISPR/Cas9 Mediated Knockout

The level of eGFP depletion was evaluated for sgRNAs delivered by ICLV and IDLV by fluorescence-activated cell sorting assay on day 7, day 14, and day 21 pt. Naïve BEK 293T-eGFP cells (not transduced with GFP virus) and non-sgRNA-expressed cells (no-sgRNA) served as controls. A percentage of GFP-positive cells remaining after transduction was recorded. The y axis represents side scatter while the x axis represents MFI. Example 3

### Example 3

### Evaluation of On-Target DNA Cleavage

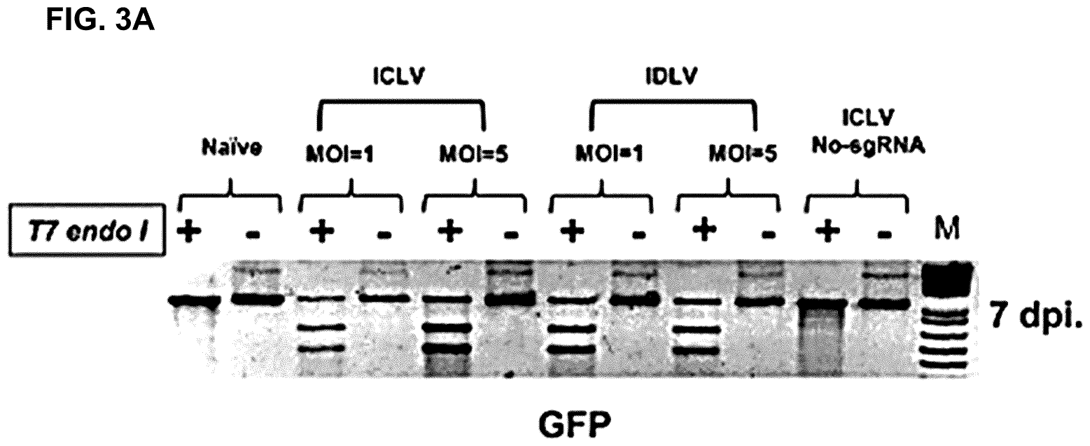

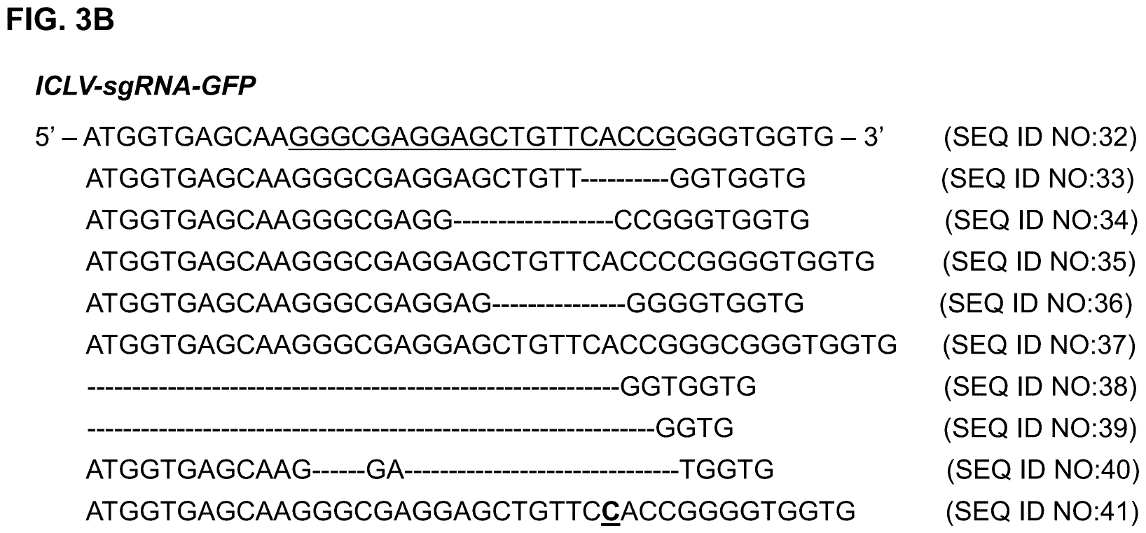

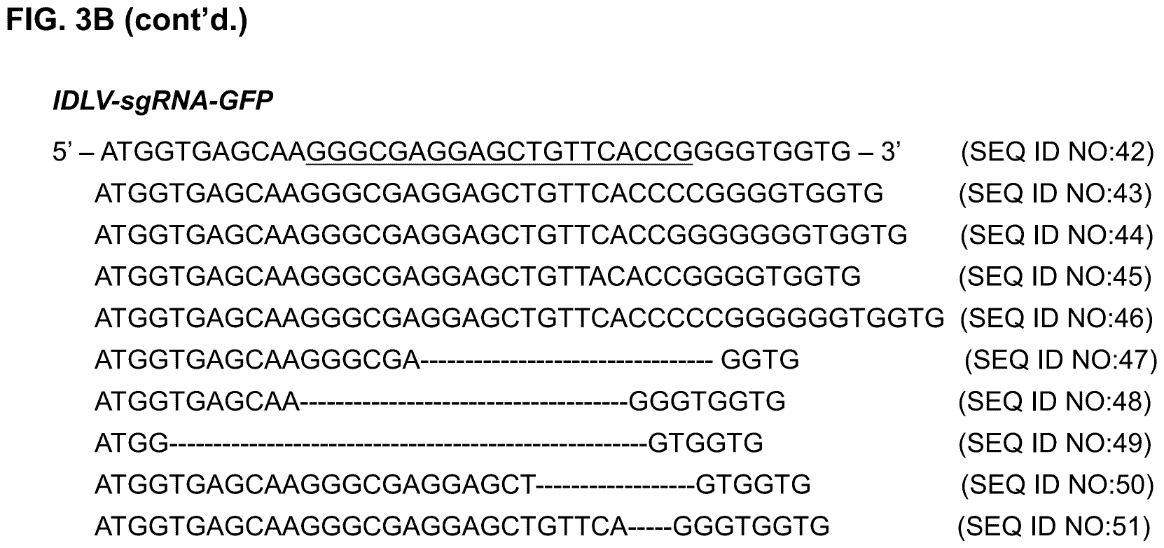

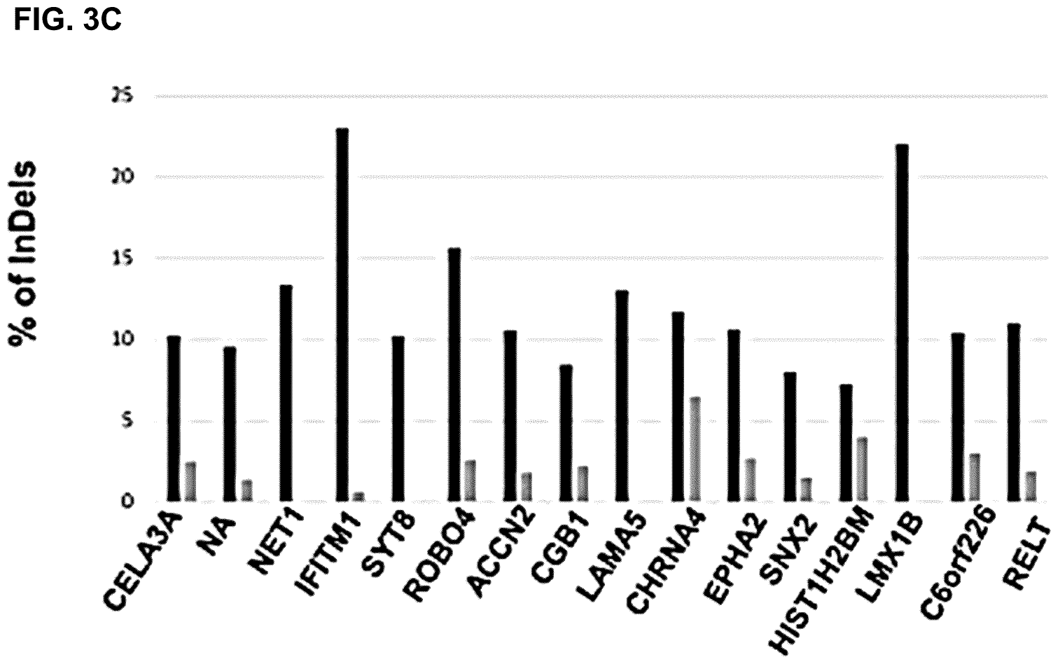

FIG. 3A shows the on-target evaluation of DNA cleavages of CRISPR/Cas9 delivered by IDLV and ICLV. The evaluation was done at 7 days pt using GFP-positive cells transduced with IDLV-sgRNA1/Cas9 and ICLV-sgRNA1/Cas9 at varying MOIs. FIG. 3B shows the use of the Sanger method to assess on target InDels of the samples transduced with either ICLV-vectors or IDLV-vectors. The rate of on-target mutations was determined at day 7. FIG. 3C illustrates the formation of InDels induced by ICLV-CRISPR/Cas9 (dark bars; see SEQ ID NO:32-SEQ ID NO: 41) and ICLV-CRISPR/Cas9 (light bars; see SEQ ID NO:42-SEQ ID NO:51). The InDels were calculated as the ratio (in percentages) of reads with mutated sequences and total reads.

### Example 4

### Evaluation of In Vivo Depletion of Target Gene

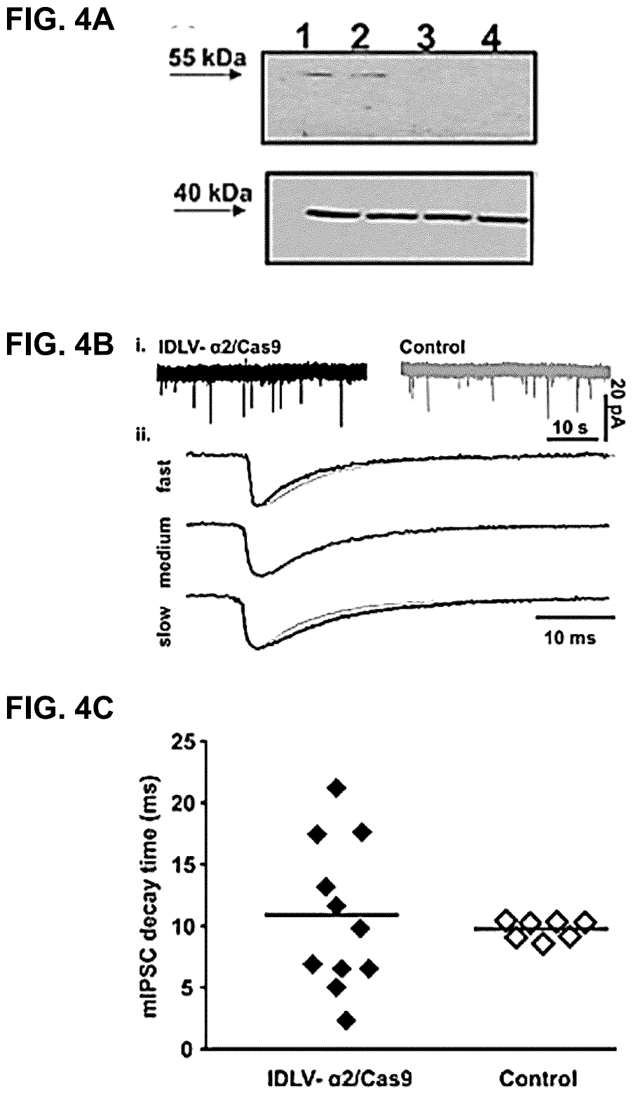

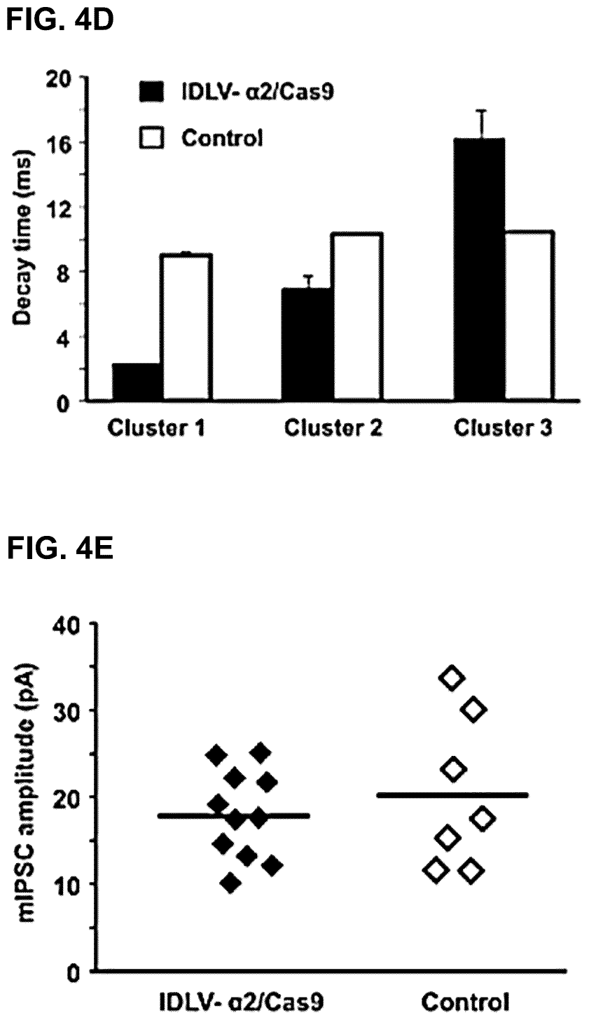

FIG. 4A show the use of western blot analysis to evaluate the efficacy of the IDLV-CRIPSR/Cas9 system to deplete the GABAA receptor α2 subunit in vivo (rats). Two control (lanes 1 and 2) and two IDLV-α2/Cas9 injected (lanes 3 and 4) animals (rats) were used. Tubulin (DM1A) antibody (˜40 kDa) was used as a loading control. FIG. 4B illustrates use of the mIPSCs method in NAc slices from control and IDLV-α2/Cas9-injected animals. Notice the similarity in event frequency at the top part of the figure (i). mIPSC averages from three different neurons (black traces) illustrate variability of mIPSC duration in slices from IDLV-α2/Cas9 animals (rats) in the bottom part of the figure (ii). For ease of comparison, each trace was overlaid onto a mIPSCs average from a single control neuron (gray trace). FIG. 4C illustrates distribution of mIPSC decay times from all recorded neurons. The horizontal black bars are centered at the mean values for each group. FIG. 4D illustrates K-means cluster analyses identify center values for slow, medium, and fast mIPSC groups of MSNs (n=1, 5, and 5 IDLV-α2/Cas9 cells in clusters 1, 2, and 3, respectively, while n=3, 2, and 2 control cells in clusters 1, 2, and 3, respectively). Finally, FIG. 4E shows the variability of mIPSC amplitudes was similar between cells in IDLV-α2/Cas9 and control groups. The horizontal black bars are centered at the mean values for each group.

### Example 5

### Validation of HDAC Knockdown

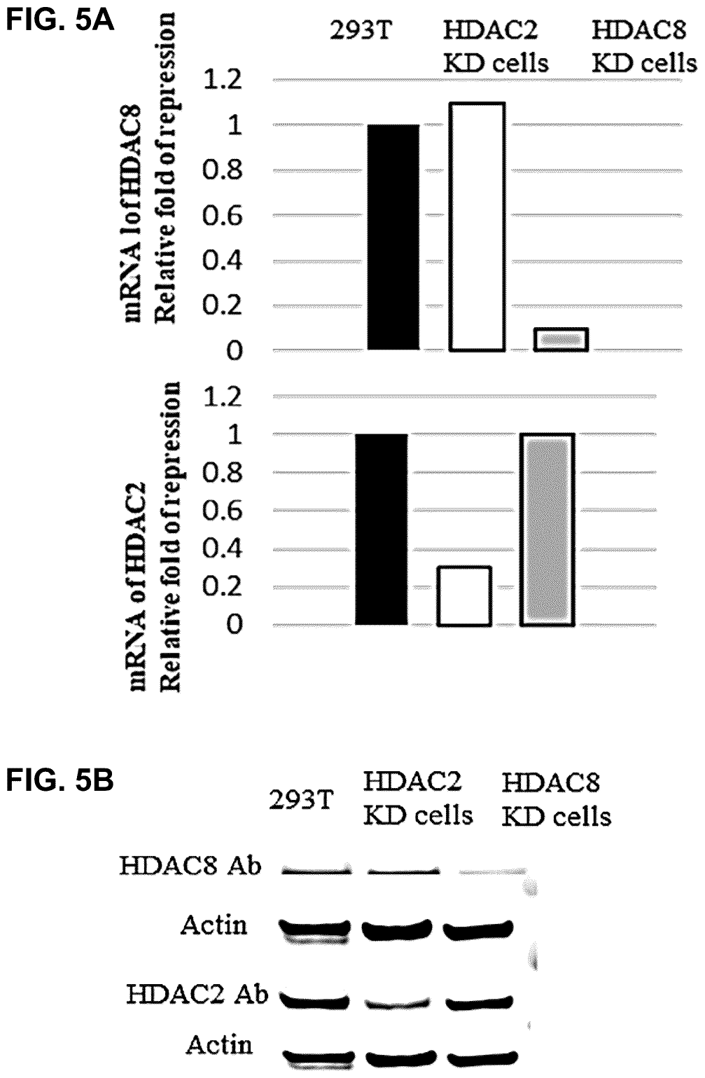

FIG. 5A-FIG. 5B show the validation of histone deacetylases (HDACs) knockdown in HEK 293T cells. HEK 293T cells were transduced with LVs harboring shRNA to the HDACs. Here the representative image highlights the knockdown achieved using shRNA-to-HDAC2 and shRNA-to-HDAC8. In FIG. 5A, the upper graph shows the mRNA levels of HDAC8 measured in naïve HEK 293T cells (black bar), in those cells transduced with HDAC2-shRNA (white bar), and in those cells transduced with HDAC8-shRNA (grey bar) using qRT analysis. In FIG. 5A, the lower panel shows the mRNA levels of HDAC2 measured in naïve HEK 293T cells (black bar), in those cells transduced with HDAC2-shRNA (white bar), and in those cells transduced with HDAC8-shRNA (grey bar) using qRT analysis. FIG. 5B shows the protein levels of HDAC8 and HDAC2 evaluated by WB performed on the cell lysates prepared from HDAC2-transduced cells and HDAC8-transduced cells. Human actin was used as a loading control. As shown, specific and robust knockdown of HDAC8 was achieved in HDAC8-targeted cells, while the levels of HDAC2 protein were efficiently reduced in HDAC2 KD cell line.

### Example 6

### Evaluation of Vectors Generated in HDAC Depleted Cells

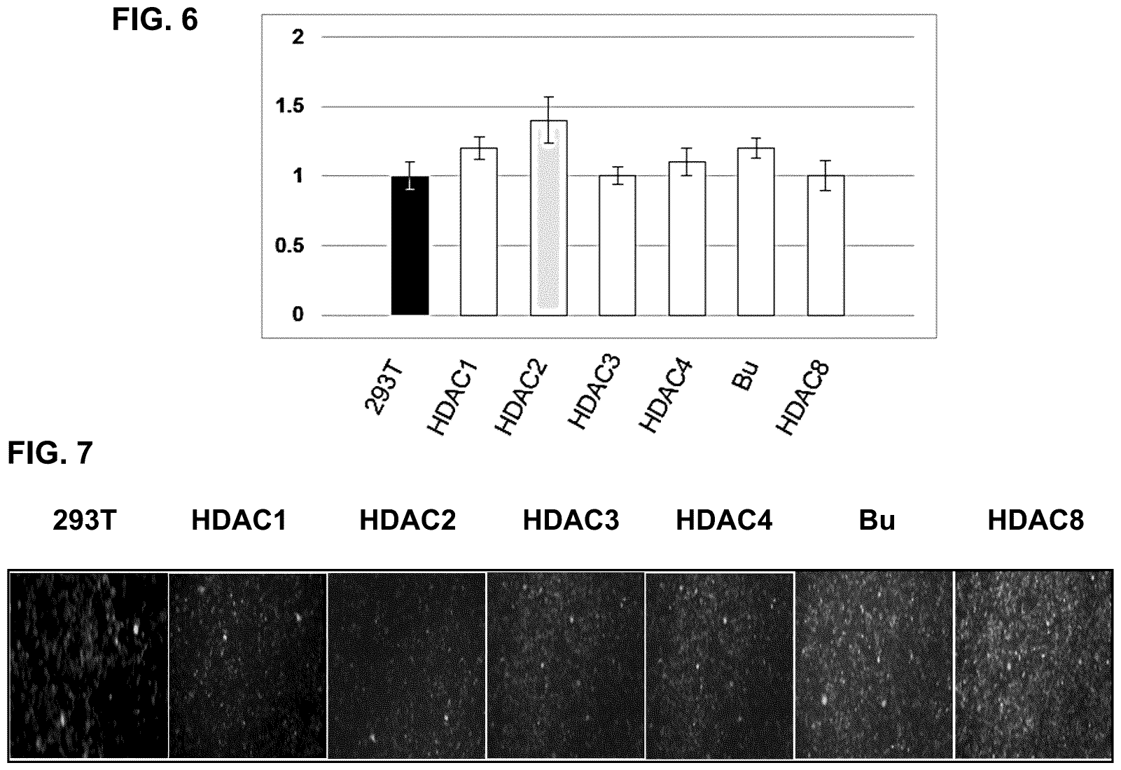

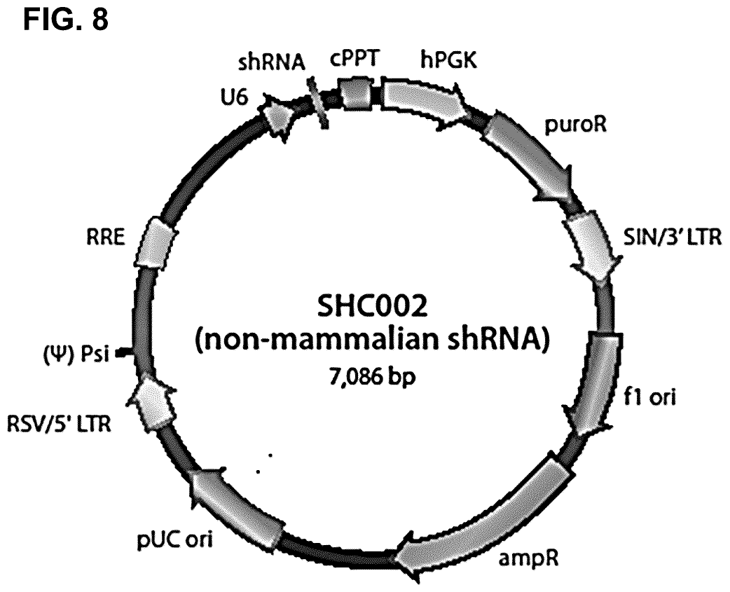

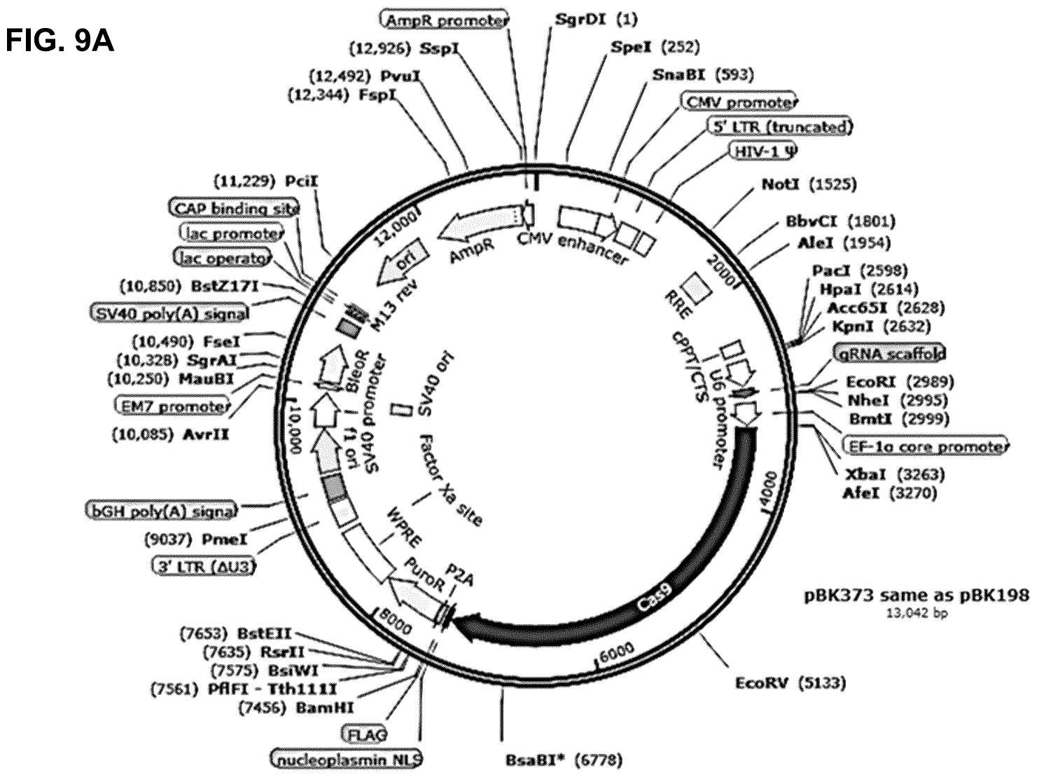

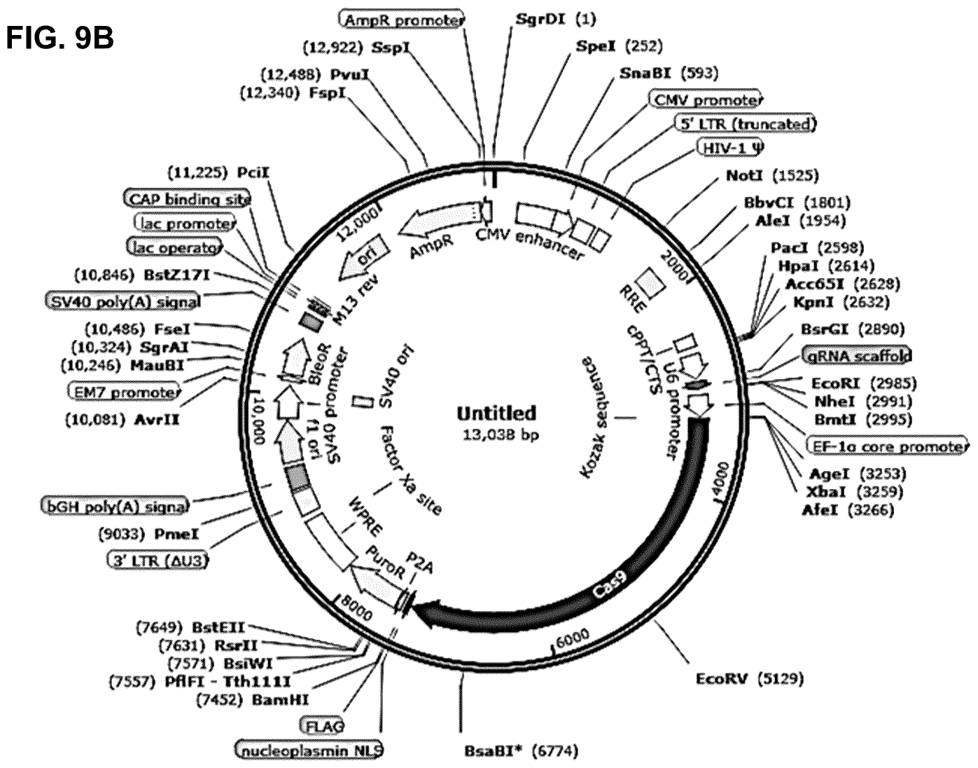

FIG. 6 shows the efficient production of IDLV vectors generated in the naïve cells, cells carrying shRNA to HDAC1, HDAC2, HDAC3, HDAC4, and HDAC8, and cells treated with sodium butyrate (Bu) at 2.5 mM. The plot reports the quantity measurement of the production using p24gag ELISA as described (Tagliafierro L, et al. (2019) J Vis Exp. (145):10.3791/59241). The further normalization is made to apply the equal viral load for transduction into HEK 293T cells.

### Example 7

### Evaluation of Transgene Expression in Vectors Generated in HDAC Depleted Cells

FIG. 7 reports the visualization of the GFP expression in the cells transduced with shRNA viruses harbored hair-pin to each of HDAC1, HDAC2, HDAC3, HDAC4, and HDAC8 as well as the cells treated with sodium butyrate (Bu) at 2.5 mM. To normalize the viral load, equal concentration of p24 content were used. The GFP expression was assessed using fluorescent microscopy method as described (Tagliafierro L, et al. (2019) J Vis Exp. (145):10.3791/59241).

### SUMMARY OF EXAMPLES

Sodium butyrate (SB) increased expression and production of the episomal genomes of both AAV and LV vectors (Kantor B, et al. (2009) Proc Natl Acad Sci USA. 106(44):18786-18791; Monahan P E, et al. (2010) Mol Ther. 18(11):1907-1916). But, SB cannot be used to enhance vector production and expression due to its broad off-target effects, including cell cycle arrest, changes in cellular differentiation and apoptotic signaling.

To overcome this technical hurdle, the data presented herein show that the production and the expression of IDLV and other non-integrating vectors like AAVs was substantially enhanced by interfering with histone deacetylase (HDAC) activity in the producer cell line. In fact, the vectors generated from HDAC-deleted cells were at substantially higher production titer and had better expression characteristics (when compared with those of the naïve or wild-type HEK 293T cells). Specifically, the Examples provided herein describe a loss-of-function screening method to inactivate HDACs (thereby removing the repressive chromatin organization that hinders and/or prevents packaging and expression capacities of episomal viruses.

Using this method, HDAC8 was identified as a key factor for negative regulation of IDLV expression and IDLV production. These data demonstrate that the inactivation of HDAC8 via specific knockdown resulted in very substantial up-regulation of IDLV genomes. These results paved the way to create the producer cell line based on HEK 293T cells deleted with HDAC8, which can be expanded to generate AAVs, LVs (IDLV and ICLV), and other viruses and vectors for efficient production of a broad-range of basic-research and therapeutic tools including CRISPR/Cas.

Moreover, the development of the novel producer cells described herein circumvents several major challenges related to gene therapy. Specifically, the disclosed optimized viral vectors (i) have high efficiency for delivery of oversized CRISPR/Cas9 components (including, for example, into non-dividing cells like the postmitotic neurons), (ii) demonstrate suitability for a broad range of cellular tropisms, (iii) show low cytotoxicity and immunogenicity, and (iv) show compatibility with advanced manufacturing techniques that are relevant to both AAV and LV platforms. The compositions and methods disclosed herein provided a gene delivery system that combines the advantages of both AAV and LV vectors while minimizing the known limitations of these vectors.

1. A viral vector, comprising: a nucleic acid sequence encoding (i) a Cas endonuclease, (ii) at least one guide RNA, and (iii) a shRNA targeting a histone deacetylase (HDAC).

2. The viral vector of claim 1, further comprising at least one transgene.

3. (canceled)

4. (canceled)

5. (canceled)

6. The viral vector of claim 1, wherein the nucleic acid sequence further encodes one or more Sp1 binding sites, one or more NFκB binding sites, or any combination thereof.

7. (canceled)

8. The viral vector of claim 7, wherein the nucleic acid sequence further comprises one or more regulatory elements, and wherein the one or more regulatory elements comprise one or more promoters, one or more primer binding sites (PBS), one or more splice donor (SD) sites, one or more splice acceptor (SA) sites, one or more central polypurine tracts (cPPT), one or more polypurine tracts (PPT), one or more Rev Response elements (RRE), one or more Woodchuck Hepatitis Virus Posttranscriptional Regulatory Elements (WPRE), one or more retroviral vector packaging elements, or any combination thereof.

9. (canceled)

10. The viral vector of claim 8, wherein the one or more promoters comprise one or more of a human Cytomegalovirus (hCMV) promoter, a core-elongation factor 1a promoter (EFS), a human U6 promoter are also included, or any combination thereof.

11. The viral vector of claim 1, further comprising a viral 3′ UTR comprising a deletion of the U3 region and/or a viral 5′ UTR.

12. (canceled)

13. The viral vector of claim 1, wherein the nucleic acid sequence further comprises a polylinker site, wherein the polylinker site comprises a pair of BsmBI sites and a unique BsrGI site.

14. (canceled)

15. (canceled)

16. The viral vector of claim 2, wherein the transgene encodes a polypeptide having transcription activation activity, transcription repression activity, transcription release factor activity, histone modification activity, nucleic acid association activity, methyltransferase activity, demethylase activity, acetyltransferase activity, deacetylase activity, or any combination thereof.

17. (canceled)

18. The viral vector of claim 2, wherein the transgene encodes a neurodegenerative disease (NDD)-relevant gene, wherein the NDD-relevant gene comprises APOE, APP, ATXN2, CHMP2B, DCTN1, FIG4, FUS, GBA, GRN, HNRNPA1, HTT, LRRK2, MATR3, OPTN, PARK7, PFN1, PRPH, PSEN1, SETX, SIGMAR1, SNCA, SOD1, SPG11, SQSTM1, TARDBP, TBK1, TBP, TRPM7, TUBA4A, UBQLN2, UCHL1, VAPB, VCP, VPS35, a fragment thereof, a variant thereof, or a chimera thereof.

19. (canceled)

20. (canceled)

21. The viral vector of claim 1, wherein the HDAC comprises HDAC1, HDAC2, HDAC3, HDAC4, HDAC6, or HDAC8, or any combination thereof.

22. (canceled)

23. The viral vector of claim 21, wherein the shRNA comprises the sequence set forth in any one of SEQ ID NO:19-SEQ ID NO:24.

24. The viral vector of claim 1, wherein the viral vector transduces a cell line to generate an HDAC^{-/-} genotype, wherein, when compared to viral vectors generated in cells having normal or wildtype HDAC expression and/or activity, the HDAC^{-/-} genotype (i) increases the packaging capacity of a generated viral vector, (ii) increases the titer of a generated viral vector, (iii) increases the expression capacity of a generated viral vector, (iv) decreases the immunogenicity and/or toxicity of a generated viral vector, (v) increases the ability of a generated viral vector to efficiently transduce, or (vi) any combination thereof.

25. The viral vector of claim 1, wherein the viral vector comprises an adeno-associated virus (AAV), an integrase-deficient lentivirus (IDLV), or integrase competent lentivirus (ICLV).

26. (canceled)

27. A method of developing HDAC depleted cells, the method comprising:
contacting one or more cells with the viral vector of claim 1;
wherein following transduction of the one or more cells, the expression and/or activity of the targeted HDAC is decreased and/or depleted.

28. (canceled)

29. The method of claim 27, wherein the HDAC depleted cells comprise a HDAC1^{-/-} genotype, a HDAC2^{-/-} genotype, a HDAC3^{-/-} genotype, a HDAC4^{-/-} genotype, a HDAC6^{-/-} genotype, a HDAC8^{-/-} genotype, or any combination thereof.

30. The method of claim 29, wherein the HDAC depleted cells comprise a HDAC8^{-/-} genotype and express HDAC1, HDAC2, HDAC3, HDAC4, HDAC6, or any combination thereof.

31. (canceled)

32. (canceled)

33. The method of claim 27, further comprising using the HDAC depleted cells to generate an optimized viral vector, wherein, when compared to a viral vector generated in cells having normal or wildtype HDAC expression and/or activity, the optimized viral vector demonstrates (i) an increase in packaging capacity, (ii) an increase in titer, (iii) a decrease in immunogenicity and/or toxicity, (iv) an increase in the ability to efficiently transduce cells, or (v) any combination thereof.

34. (canceled)

35. (canceled)

36. (canceled)

37. (canceled)

38. (canceled)

39. The method of claim 33, wherein the optimized viral vector comprises an ICLV vector, an IDLV vector, or an AAV vector.

40. (canceled)

41. A method of treating a subject, the method comprising:
administering a therapeutically effective amount of the optimized viral vector of claim 34 to a subject in need thereof.

42. (canceled)

43. The method of claim 41, wherein the subject in need thereof has a genetic defect or disorder that affects a neurodegenerative disease (NDD)-related gene, wherein the NDD-relevant gene comprises APOE, APP, ATXN2, CHMP2B, DCTN1, FIG4, FUS, GBA, GRN, HNRNPA1, HTT, LRRK2, MATR3, OPTN, PARK7, PFN1, PRPH, PSEN1, SETX, SIGMAR1, SNCA, SOD1, SPG11, SQSTM1, TARDBP, TBK1, TBP, TRPM7, TUBA4A, UBQLN2, UCHL1, VAPB, VCP, VPS35, a fragment thereof, a variant thereof, or a chimera thereof.

44. (canceled)


SEQUENCE LISTING (65 sequences)

SEQ ID NO: 1 - DNA, 13038 bp, synthetic construct
  source 1..13038: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgggcgggacgttaacggggcggaacggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagacgtgtacacgtctctgttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagcgctgccaccatggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaagattacaaagacgatgacgataagggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 2 - DNA, 14488 bp, synthetic construct
  source 1..14488: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgggcgggacgttaacggggcggaacggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggggcgaggagctgttcaccggttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagcgctgccaccatggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaagattacaaagacgatgacgataagggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacatggcgcagacgcagggcacccggaggaaagtctgttactactacgacggggatgttggaaattactattatggacaaggccacccaatgaagcctcaccgaatccgcatgactcataatttgctgctcaactatggtctctaccgaaaaatggaaatctatcgccctcacaaagccaatgctgaggagatgaccaagtaccacagcgatgactacattaaattcttgcgctccatccgtccagataacatgtcggagtacagcaagcagatgcagagattcaacgttggtgaggactgtccagtattcgatggcctgtttgagttctgtcagttgtctactggtggttctgtggcaagtgctgtgaaacttaataagcagcagacggacatcgctgtgaattgggctgggggcctgcaccatgcaaagaagtccgaggcatctggcttctgttacgtcaatgatatcgtcttggccatcctggaactgctaaagtatcaccagagggtgctgtacattgacattgatattcaccatggtgacggcgtggaagaggccttctacaccacggaccgggtcatgactgtgtcctttcataagtatggagagtacttcccaggaactggggacctacgggatatcggggctggcaaaggcaagtattatgctgttaactacccgctccgagacgggattgatgacgagtcctatgaggccattttcaagccggtcatgtccaaagtaatggagatgttccagcctagtgcggtggtcttacagtgtggctcagactccctatctggggatcggttaggttgcttcaatctaactatcaaaggacacgccaagtgtgtggaatttgtcaagagctttaacctgcctatgctgatgctgggaggcggtggttacaccattcgtaacgttgcccggtgctggacatatgagacagctgtggccctggatacggagatccctaatgagcttccatacaatgactactttgaatactttggaccagatttcaagctccacatcagtccttccaatatgactaaccagaacacgaatgagtacctggagaagatcaaacagcgactgtttgagaaccttagaatgctgccgcacgcacctggggtccaaatgcaggcgattcctgaggacgccatccctgaggagagtggcgatgaggacgaagacgaccctgacaagcgcatctcgatctgctcctctgacaaacgaattgcctgtgaggaagagttctccgattctgaagaggagggagaggggggccgcaagaactcttccaacttcaaaaaagccaagagagtcaaaacagaggatgaaaaagagaaagacccagaggagaagaaagaagtcaccgaagaggagaaaaccaaggaggagaagccagaagccaaaggggtcaaggaggaggtcaagttggcctga

SEQ ID NO: 3 - DNA, 717 bp, synthetic construct
  source 1..717: mol_type = other DNA
atgagcaagggcgaggaactgttcactggcgtggtcccaattctcgtggaactggatggcgatgtgaatgggcacaaattttctgtcagcggagagggtgaaggtgatgccacatacggaaagctcaccctgaaattcatctgcaccactggaaagctccctgtgccatggccaacactggtcactaccttctcttatggcgtgcagtgcttttccagatacccagaccatatgaagcagcatgactttttcaagagcgccatgcccgagggctatgtgcaggagagaaccatctttttcaaagatgacgggaactacaagacccgcgctgaagtcaagttcgaaggtgacaccctggtgaatagaatcgagctgaagggcattgactttaaggaggatggaaacattctcggccacaagctggaatacaactataactcccacaatgtgtacatcatggccgacaagcaaaagaatggcatcaaggtcaacttcaagatcagacacaacattgaggatggatccgtgcagctggccgaccattatcaacagaacactccaatcggcgacggccctgtgctcctcccagacaaccattacctgtccacccagtctgccctgtctaaagatcccaacgaaaagagagaccacatggtcctgctggagtttgtgaccgctgctgggatcacacatggcatggacgagctgtacaagtga

SEQ ID NO: 4 - protein, 238 aa, synthetic construct
MSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 5 - DNA, 25 bp, synthetic construct
  source 1..25: mol_type = other DNA
caccggggcgaggagctgttcaccg

SEQ ID NO: 6 - DNA, 25 bp, synthetic construct
  source 1..25: mol_type = other DNA
aaaccggtgaacagctcctcgcccc

SEQ ID NO: 7 - DNA, 1446 bp, Homo sapiens
  source 1..1446: mol_type = other DNA
atggcgcagacgcagggcacccggaggaaagtctgttactactacgacggggatgttggaaattactattatggacaaggccacccaatgaagcctcaccgaatccgcatgactcataatttgctgctcaactatggtctctaccgaaaaatggaaatctatcgccctcacaaagccaatgctgaggagatgaccaagtaccacagcgatgactacattaaattcttgcgctccatccgtccagataacatgtcggagtacagcaagcagatgcagagattcaacgttggtgaggactgtccagtattcgatggcctgtttgagttctgtcagttgtctactggtggttctgtggcaagtgctgtgaaacttaataagcagcagacggacatcgctgtgaattgggctgggggcctgcaccatgcaaagaagtccgaggcatctggcttctgttacgtcaatgatatcgtcttggccatcctggaactgctaaagtatcaccagagggtgctgtacattgacattgatattcaccatggtgacggcgtggaagaggccttctacaccacggaccgggtcatgactgtgtcctttcataagtatggagagtacttcccaggaactggggacctacgggatatcggggctggcaaaggcaagtattatgctgttaactacccgctccgagacgggattgatgacgagtcctatgaggccattttcaagccggtcatgtccaaagtaatggagatgttccagcctagtgcggtggtcttacagtgtggctcagactccctatctggggatcggttaggttgcttcaatctaactatcaaaggacacgccaagtgtgtggaatttgtcaagagctttaacctgcctatgctgatgctgggaggcggtggttacaccattcgtaacgttgcccggtgctggacatatgagacagctgtggccctggatacggagatccctaatgagcttccatacaatgactactttgaatactttggaccagatttcaagctccacatcagtccttccaatatgactaaccagaacacgaatgagtacctggagaagatcaaacagcgactgtttgagaaccttagaatgctgccgcacgcacctggggtccaaatgcaggcgattcctgaggacgccatccctgaggagagtggcgatgaggacgaagacgaccctgacaagcgcatctcgatctgctcctctgacaaacgaattgcctgtgaggaagagttctccgattctgaagaggagggagaggggggccgcaagaactcttccaacttcaaaaaagccaagagagtcaaaacagaggatgaaaaagagaaagacccagaggagaagaaagaagtcaccgaagaggagaaaaccaaggaggagaagccagaagccaaaggggtcaaggaggaggtcaagttggcc

SEQ ID NO: 8 - DNA, 1467 bp, Homo sapiens
  source 1..1467: mol_type = other DNA
atggcgtacagtcaaggaggcggcaaaaaaaaagtctgctactactacgacggtgatattggaaattattattatggacagggtcatcccatgaagcctcatagaatccgcatgacccataacttgctgttaaattatggcttatacagaaaaatggaaatatataggccccataaagccactgccgaagaaatgacaaaatatcacagtgatgagtatatcaaatttctacggtcaataagaccagataacatgtctgagtatagtaagcagatgcagagatttaatgttggagaagattgtccagtgtttgatggactctttgagttttgtcagctctcaactggcggttcagttgctggagctgtgaagttaaaccgacaacagactgatatggctgttaattgggctggaggattacatcatgctaagaaatcagaagcatcaggattctgttacgttaatgatattgtgcttgccatccttgaattactaaagtatcatcagagagtcttatatattgatatagatattcatcatggtgatggtgttgaagaagctttttatacaacagatcgtgtaatgacggtatcattccataaatatggggaatactttcctggcacaggagacttgagggatattggtgctggaaaaggcaaatactatgctgtcaattttccaatgagagatggtatagatgatgagtcatatgggcagatatttaagcctattatctcaaaggtgatggagatgtatcaacctagtgctgtggtattacagtgtggtgcagactcattatctggtgatagactgggttgtttcaatctaacagtcaaaggtcatgctaaatgtgtagaagttgtaaaaacttttaacttaccattactgatgcttggaggaggtggctacacaatccgtaatgttgctcgatgttggacatatgagactgcagttgcccttgattgtgagattcccaatgagttgccatataatgattactttgagtattttggaccagacttcaaactgcatattagtccttcaaacatgacaaaccagaacactccagaatatatggaaaagataaaacagcgtttgtttgaaaatttgcgcatgttacctcatgcacctggtgtccagatgcaagctattccagaagatgctgttcatgaagacagtggagatgaagatggagaagatccagacaagagaatttctattcgagcatcagacaagcggatagcttgtgatgaagaattctcagattctgaggatgaaggagaaggaggtcgaagaaatgtggctgatcataagaaaggagcaaagaaagctagaattgaagaagataagaaagaaacagaggacaaaaaaacagacgttaaggaagaagataaatccaaggacaacagtggtgaaaaaacagataccaaaggaaccaaatcagaacagctcagcaacccctga

SEQ ID NO: 9 - DNA, 1287 bp, Homo sapiens
  source 1..1287: mol_type = other DNA
atggccaagaccgtggcctatttctacgaccccgacgtgggcaacttccactacggagctggacaccctatgaagccccatcgcctggcattgacccatagcctggtcctgcattacggtctctataagaagatgatcgtcttcaagccataccaggcctcccaacatgacatgtgccgcttccactccgaggactacattgacttcctgcagagagtcagccccaccaatatgcaaggcttcaccaagagtcttaatgccttcaacgtaggcgatgactgcccagtgtttcccgggctctttgagttctgctcgcgttacacaggcgcatctctgcaaggagcaacccagctgaacaacaagatctgtgatattgccattaactgggctggtggtctgcaccatgccaagaagtttgaggcctctggcttctgctatgtcaacgacattgtgattggcatcctggagctgctcaagtaccaccctcgggtgctctacattgacattgacatccaccatggtgacggggttcaagaagctttctacctcactgaccgggtcatgacggtgtccttccacaaatacggaaattacttcttccctggcacaggtgacatgtatgaagtcggggcagagagtggccgctactactgtctgaacgtgcccctgcgggatggcattgatgaccagagttacaagcaccttttccagccggttatcaaccaggtagtggacttctaccaacccacgtgcattgtgctccagtgtggagctgactctctgggctgtgatcgattgggctgctttaacctcagcatccgagggcatggggaatgcgttgaatatgtcaagagcttcaatatccctctactcgtgctgggtggtggtggttatactgtccgaaatgttgcccgctgctggacatatgagacatcgctgctggtagaagaggccattagtgaggagcttccctatagtgaatacttcgagtactttgccccagacttcacacttcatccagatgtcagcacccgcatcgagaatcagaactcacgccagtatctggaccagatcctccagacaatctttgaaaacctgaagatgctgaaccatgcacctagtgtccagattcatgacgtgcctgcagacctcctgacctatgacaggactgatgaggctgatgcagaggagaggggtcctgaggagaactatagcaggccagaggcacccaatgagttctatgatggagaccatgacaatgacaaggaaagcgatgtggagatttaa

SEQ ID NO: 10 - DNA, 3270 bp, Homo sapiens
  source 1..3270: mol_type = other DNA
atgagctcccaaagccatccagatggactttctggccgagaccagccagtggagctgctgaatcctgcccgcgtgaaccacatgcccagcacggtggatgtggccacggcgctgcctctgcaagtggccccctcggcagtgcccatggacctgcgcctggaccaccagttctcactgcctgtggcagagccggccctgcgggagcagcagctgcagcaggagctcctggcgctcaagcagaagcagcagatccagaggcagatcctcatcgctgagttccagaggcagcacgagcagctctcccggcagcacgaggcgcagctccacgagcacatcaagcaacaacaggagatgctggccatgaagcaccagcaggagctgctggaacaccagcggaagctggagaggcaccgccaggagcaggagctggagaagcagcaccgggagcagaagctgcagcagctcaagaacaaggagaagggcaaagagagtgccgtggccagcacagaagtgaagatgaagttacaagaatttgtcctcaataaaaagaaggcgctggcccaccggaatctgaaccactgcatttccagcgaccctcgctactggtacgggaaaacgcagcacagttcccttgaccagagttctccaccccagagcggagtgtcgacctcctataaccacccggtcctgggaatgtacgacgccaaagatgacttccctcttaggaaaacagcttctgaaccgaatctgaaattacggtccaggctaaagcagaaagtggccgaaagacggagcagccccctgttacgcaggaaagacgggccagtggtcactgctctaaaaaagcgtccgttggatgtcacagactccgcgtgcagcagcgccccaggctccggacccagctcacccaacaacagctccgggagcgtcagcgcggagaacggtatcgcgcccgccgtccccagcatcccggcggagacgagtttggcgcacagacttgtggcacgagaaggctcggccgctccacttcccctctacacatcgccatccttgcccaacatcacgctgggcctgcctgccaccggcccctctgcgggcacggcgggccagcaggacgccgagagactcacccttcccgccctccagcagaggctctcccttttccccggcacccacctcactccctacctgagcacctcgcccttggagcgggacggaggggcagcgcacagccctcttctgcagcacatggtcttactggagcagccgccggcacaagcacccctcgtcacagactggtatctttcaggcctgggagcactgcccctccacgcacagtccttggttggtgcagaccgggtgtccccctccatccacaagctgcggcagcaccgcccactggggcggacccagtcggccccgctgccccagaacgcccaggctctgcagcacctggtcatccagcagcagcatcagcagtttctggagaaacacaagcagcagttccagcagcagcaactgcagatgaacaagatcatccccaagccaagcgagccagcccggcagccggagagccacccggaggagacggaggaggagctccgtgagcaccaggctctgctggacgagccctacctggaccggctgccggggcagaaggaggcgcacgcacaggccggcgtgcaggtgaagcaggagcccattgagagcgatgaggaagaggcagagcccccacgggaggtggagccgggccagcgccagcccagtgagcaggagctgctcttcagacagcaagccctcctgctggagcagcagcggatccaccagctgaggaactaccaggcgtccatggaggccgccggcatccccgtgtccttcggcggccacaggcctctgtcccgggcgcagtcctcacccgcgtctgccaccttccccgtgtctgtgcaggagccccccaccaagccgaggttcacgacaggcctcgtgtatgacacgctgatgctgaagcaccagtgcacctgcgggagtagcagcagccaccccgagcacgccgggaggatccagagcatctggtcccgcctgcaggagacgggcctccggggcaaatgcgagtgcatccgcggacgcaaggccaccctggaggagctacagacggtgcactcggaagcccacaccctcctgtatggcacgaaccccctcaaccggcagaaactggacagtaagaaacttctaggctcgctcgcctccgtgttcgtccggctcccttgcggtggtgttggggtggacagtgacaccatatggaacgaggtgcactcggcgggggcagcccgcctggctgtgggctgcgtggtagagctggtcttcaaggtggccacaggggagctgaagaatggctttgctgtggtccgcccccctggacaccatgcggaggagagcacgcccatgggcttttgctacttcaactccgtggccgtggcagccaagcttctgcagcagaggttgagcgtgagcaagatcctcatcgtggactgggacgtgcaccatggaaacgggacccagcaggctttctacagcgaccccagcgtcctgtacatgtccctccaccgctacgacgatgggaacttcttcccaggcagcggggctcctgatgaggtgggcacagggcccggcgtgggtttcaacgtcaacatggctttcaccggcggcctggacccccccatgggagacgctgagtacttggcggccttcagaacggtggtcatgccgatcgccagcgagtttgccccggatgtggtgctggtgtcatcaggcttcgatgccgtggagggccaccccacccctcttgggggctacaacctctccgccagatgcttcgggtacctgacgaagcagctgatgggcctggctggcggccggattgtcctggccctcgagggaggccacgacctgaccgccatttgcgacgcctcggaagcatgtgtttctgccttgctgggaaacgagcttgatcctctcccagaaaaggttttacagcaaagacccaatgcaaacgctgtccgttccatggagaaagtcatggagatccacagcaagtactggcgctgcctgcagcgcacaacctccacagcggggcgttctctgatcgaggctcagacttgcgagaacgaagaagccgagacggtcaccgccatggcctcgctgtccgtgggcgtgaagcccgccgaaaagagaccagatgaggagcccatggaagaggagccgcccctgtag

SEQ ID NO: 11 - DNA, 3690 bp, Homo sapiens
  source 1..3690: mol_type = other DNA
atgagtggagcgaaccgcggcaggggccaagcctcctcaactatgacctcaaccggccaggattccaccacaaccaggcagcgaagaagtaggcagaacccccagtcgccccctcaggactccagtgtcacttcgaagcgaaatattaaaaagggagccgttccccgctctatccccaatctagcggaggtaaagaagaaaggcaaaatgaagaagctcggccaagcaatggaagaagacctaatcgtgggactgcaagggatggatctgaaccttgaggctgaagcactggctggcactggcttggtgttggatgagcagttaaatgaattccattgcctctgggatgacagcttcccggaaggccctgagcggctccatgccatcaaggagcaactgatccaggagggcctcctagatcgctgcgtgtcctttcaggcccggtttgctgaaaaggaagagctgatgttggttcacagcctagaatatattgatctgatggaaacaacccagtacatgaatgagggagaactccgtgtcctagcagacacctacgactcagtttatctgcatccgaactcatactcctgtgcctgcctggcctcaggctctgtcctcaggctggtggatgcggtcctgggggctgagatccggaatggcatggccatcattaggcctcctggacatcacgcccagcacagtcttatggatggctattgcatgttcaaccacgtggctgtggcagcccgctatgctcaacagaaacaccgcatccggagggtccttatcgtagattgggatgtgcaccacggtcaaggaacacagttcaccttcgaccaggaccccagtgtcctctatttctccatccaccgctacgagcagggtaggttctggccccacctgaaggcctctaactggtccaccacaggtttcggccaaggccaaggatataccatcaatgtgccttggaaccaggtggggatgcgggatgctgactacattgctgctttcctgcacgtcctgctgccagtcgccctcgagttccagcctcagctggtcctggtggctgctggatttgatgccctgcaaggggaccccaagggtgagatggccgccactccggcagggttcgcccagctaacccacctgctcatgggtctggcaggaggcaagctgatcctgtctctggagggtggctacaacctccgcgccctggctgaaggcgtcagtgcttcgctccacacccttctgggagacccttgccccatgctggagtcacctggtgccccctgccggagtgcccaggcttcagtttcctgtgctctggaagcccttgagcccttctgggaggttcttgtgagatcaactgagaccgtggagagggacaacatggaggaggacaatgtagaggagagcgaggaggaaggaccctgggagccccctgtgctcccaatcctgacatggccagtgctacagtctcgcacagggctggtctatgaccaaaatatgatgaatcactgcaacttgtgggacagccaccaccctgaggtaccccagcgcatcttgcggatcatgtgccgtctggaggagctgggccttgccgggcgctgcctcaccctgacaccgcgccctgccacagaggctgagctgctcacctgtcacagtgctgagtacgtgggtcatctccgggccacagagaaaatgaaaacccgggagctgcaccgtgagagttccaactttgactccatctatatctgccccagtaccttcgcctgtgcacagcttgccactggcgctgcctgccgcctggtggaggctgtgctctcaggagaggttctgaatggtgctgctgtggtgcgtcccccaggacaccacgcagagcaggatgcagcttgcggtttttgctttttcaactctgtggctgtggctgctcgccatgcccagactatcagtgggcatgccctacggatcctgattgtggattgggatgtccaccacggtaatggaactcagcacatgtttgaggatgaccccagtgtgctatatgtgtccctgcaccgctatgatcatggcaccttcttccccatgggggatgagggtgccagcagccagatcggccgggctgcgggcacaggcttcaccgtcaacgtggcatggaacgggccccgcatgggtgatgctgactacctagctgcctggcatcgcctggtgcttcccattgcctacgagtttaacccagaactggtgctggtctcagctggctttgatgctgcacggggggatccgctggggggctgccaggtgtcacctgagggttatgcccacctcacccacctgctgatgggccttgccagtggccgcattatccttatcctagagggtggctataacctgacatccatctcagagtccatggctgcctgcactcgctccctccttggagacccaccacccctgctgaccctgccacggcccccactatcaggggccctggcctcaatcactgagaccatccaagtccatcgcagatactggcgcagcttacgggtcatgaaggtagaagacagagaaggaccctccagttctaagttggtcaccaagaaggcaccccaaccagccaaacctaggttagctgagcggatgaccacacgagaaaagaaggttctggaagcaggcatggggaaagtcacctcggcatcatttggggaagagtccactccaggccagactaactcagagacagctgtggtggccctcactcaggaccagccctcagaggcagccacagggggagccactctggcccagaccatttctgaggcagccattgggggagccatgctgggccagaccacctcagaggaggctgtcgggggagccactccggaccagaccacctcagaggagactgtgggaggagccattctggaccagaccacctcagaggatgctgttgggggagccacgctgggccagactacctcagaggaggctgtaggaggagctacactggcccagaccacctcggaggcagccatggagggagccacactggaccagactacgtcagaggaggctccagggggcaccgagctgatccaaactcctctagcctcgagcacagaccaccagacccccccaacctcacctgtgcagggaactacaccccagatatctcccagtacactgattgggagtctcaggaccttggagctaggcagcgaatctcagggggcctcagaatctcaggccccaggagaggagaacctactaggagaggcagctggaggtcaggacatggctgattcgatgctgatgcagggatctaggggcctcactgatcaggccatattttatgctgtgacaccactgccctggtgtccccatttggtggcagtatgccccatacctgcagcaggcctagacgtgacccaaccttgtggggactgtggaacaatccaagagaattgggtgtgtctctcttgctatcaggtctactgtggtcgttacatcaatggccacatgctccaacaccatggaaattctggacacccgctggtcctcagctacatcgacctgtcagcctggtgttactactgtcaggcctatgtccaccaccaggctctcctagatgtgaagaacatcgcccaccagaacaagtttggggaggatatgccccacccacactaa

SEQ ID NO: 12 - DNA, 1134 bp, Homo sapiens
  source 1..1134: mol_type = other DNA
atggaggagccggaggaaccggcggacagtgggcagtcgctggtcccggtttatatctatagtcccgagtatgtcagtatgtgtgactccctggccaagatccccaaacgggccagtatggtgcattctttgattgaagcatatgcactgcataagcagatgaggatagttaagcctaaagtggcctccatggaggagatggccaccttccacactgatgcttatctgcagcatctccagaaggtcagccaagagggcgatgatgatcatccggactccatagaatatgggctaggttatgactgcccagccactgaagggatatttgactatgcagcagctataggaggggctacgatcacagctgcccaatgcctgattgacggaatgtgcaaagtagcaattaactggtctggagggtggcatcatgcaaagaaagatgaagcatctggtttttgttatctcaatgatgctgtcctgggaatattacgattgcgacggaaatttgagcgtattctctacgtggatttggatctgcaccatggagatggtgtagaagacgcattcagtttcacctccaaagtcatgaccgtgtccctgcacaaattctccccaggatttttcccaggaacaggtgacgtgtctgatgttggcctagggaagggacggtactacagtgtaaatgtgcccattcaggatggcatacaagatgaaaaatattaccagatctgtgaaagtgtactaaaggaagtataccaagcctttaatcccaaagcagtggtcttacagctgggagctgacacaatagctggggatcccatgtgctcctttaacatgactccagtgggaattggcaagtgtcttaagtacatccttcaatggcagttggcaacactcattttgggaggaggaggctataaccttgccaacacggctcgatgctggacatacttgaccggggtcatcctagggaaaacactatcctctgagatcccagatcatgagtttttcacagcatatggtcctgattatgtgctggaaatcacgccaagctgccggccagaccgcaatgagccccaccgaatccaacaaatcctcaactacatcaaagggaatctgaagcatgtggtctag

SEQ ID NO: 13 - protein, 482 aa, Homo sapiens
MAQTQGTRRKVCYYYDGDVGNYYYGQGHPMKPHRIRMTHNLLLNYGLYRKMEIYRPHKANAEEMTKYHSDDYIKFLRSIRPDNMSEYSKQMQRFNVGEDCPVFDGLFEFCQLSTGGSVASAVKLNKQQTDIAVNWAGGLHHAKKSEASGFCYVNDIVLAILELLKYHQRVLYIDIDIHHGDGVEEAFYTTDRVMTVSFHKYGEYFPGTGDLRDIGAGKGKYYAVNYPLRDGIDDESYEAIFKPVMSKVMEMFQPSAVVLQCGSDSLSGDRLGCFNLTIKGHAKCVEFVKSFNLPMLMLGGGGYTIRNVARCWTYETAVALDTEIPNELPYNDYFEYFGPDFKLHISPSNMTNQNTNEYLEKIKQRLFENLRMLPHAPGVQMQAIPEDAIPEESGDEDEDDPDKRISICSSDKRIACEEEFSDSEEEGEGGRKNSSNFKKAKRVKTEDEKEKDPEEKKEVTEEEKTKEEKPEAKGVKEEVKLA

SEQ ID NO: 14 - protein, 488 aa, Homo sapiens
MAYSQGGGKKKVCYYYDGDIGNYYYGQGHPMKPHRIRMTHNLLLNYGLYRKMEIYRPHKATAEEMTKYHSDEYIKFLRSIRPDNMSEYSKQMQRFNVGEDCPVFDGLFEFCQLSTGGSVAGAVKLNRQQTDMAVNWAGGLHHAKKSEASGFCYVNDIVLAILELLKYHQRVLYIDIDIHHGDGVEEAFYTTDRVMTVSFHKYGEYFPGTGDLRDIGAGKGKYYAVNFPMRDGIDDESYGQIFKPIISKVMEMYQPSAVVLQCGADSLSGDRLGCFNLTVKGHAKCVEVVKTFNLPLLMLGGGGYTIRNVARCWTYETAVALDCEIPNELPYNDYFEYFGPDFKLHISPSNMTNQNTPEYMEKIKQRLFENLRMLPHAPGVQMQAIPEDAVHEDSGDEDGEDPDKRISIRASDKRIACDEEFSDSEDEGEGGRRNVADHKKGAKKARIEEDKKETEDKKTDVKEEDKSKDNSGEKTDTKGTKSEQLSNP

SEQ ID NO: 15 - protein, 428 aa, Homo sapiens [sequence omitted]

SEQ ID NO: 16 - protein, 1089 aa, Homo sapiens
MSSQSHPDGLSGRDQPVELLNPARVNHMPSTVDVATALPLQVAPSAVPMDLRLDHQFSLPVAEPALREQQLQQELLALKQKQQIQRQILIAEFQRQHEQLSRQHEAQLHEHIKQQQEMLAMKHQQELLEHQRKLERHRQEQELEKQHREQKLQQLKNKEKGKESAVASTEVKMKLQEFVLNKKKALAHRNLNHCISSDPRYWYGKTQHSSLDQSSPPQSGVSTSYNHPVLGMYDAKDDFPLRKTASEPNLKLRSRLKQKVAERRSSPLLRRKDGPVVTALKKRPLDVTDSACSSAPGSGPSSPNNSSGSVSAENGIAPAVPSIPAETSLAHRLVAREGSAAPLPLYTSPSLPNITLGLPATGPSAGTAGQQDAERLTLPALQQRLSLFPGTHLTPYLSTSPLERDGGAAHSPLLQHMVLLEQPPAQAPLVTDWYLSGLGALPLHAQSLVGADRVSPSIHKLRQHRPLGRTQSAPLPQNAQALQHLVIQQQHQQFLEKHKQQFQQQQLQMNKIIPKPSEPARQPESHPEETEEELREHQALLDEPYLDRLPGQKEAHAQAGVQVKQEPIESDEEEAEPPREVEPGQRQPSEQELLFRQQALLLEQQRIHQLRNYQASMEAAGIPVSFGGHRPLSRAQSSPASATFPVSVQEPPTKPRFTTGLVYDTLMLKHQCTCGSSSSHPEHAGRIQSIWSRLQETGLRGKCECIRGRKATLEELQTVHSEAHTLLYGTNPLNRQKLDSKKLLGSLASVFVRLPCGGVGVDSDTIWNEVHSAGAARLAVGCVVELVFKVATGELKNGFAVVRPPGHHAEESTPMGFCYFNSVAVAAKLLQQRLSVSKILIVDWDVHHGNGTQQAFYSDPSVLYMSLHRYDDGNFFPGSGAPDEVGTGPGVGFNVNMAFTGGLDPPMGDAEYLAAFRTVVMPIASEFAPDVVLVSSGFDAVEGHPTPLGGYNLSARCFGYLTKQLMGLAGGRIVLALEGGHDLTAICDASEACVSALLGNELDPLPEKVLQQRPNANAVRSMEKVMEIHSKYWRCLQRTTSTAGRSLIEAQTCENEEAETVTAMASLSVGVKPAEKRPDEEPMEEEPPL

SEQ ID NO: 17 - protein, 1260 aa, Homo sapiens
MEKKSLSARVPTGRQKLGGASQGRSLRKGLRPMSGANRGRGQASSTMTSTGQDSTTTRQRRSRQNPQSPPQDSSVTSKRNIKKGAVPRSIPNLAEVKKKGKMKKLGQAMEEDLIVGLQGMDLNLEAEALAGTGLVLDEQLNEFHCLWDDSFPEGPERLHAIKEQLIQEGLLDRCVSFQARFAEKEELMLVHSLEYIDLMETTQYMNEGELRVLADTYDSVYLHPNSYSCACLASGSVLRLVDAVLGAEIRNGMAIIRPPGHHAQHSLMDGYCMFNHVAVAARYAQQKHRIRRVLIVDWDVHHGQGTQFTFDQDPSVLYFSIHRYEQGRFWPHLKASNWSTTGFGQGQGYTINVPWNQVGMRDADYIAAFLHVLLPVALEFQPQLVLVAAGFDALQGDPKGEMAATPAGFAQLTHLLMGLAGGKLILSLEGGYNLRALAEGVSASLHTLLGDPCPMLESPGAPCRSAQASVSCALEALEPFWEVLVRSTETVERDNMEEDNVEESEEEGPWEPPVLPILTWPVLQSRTGLVYDQNMMNHCNLWDSHHPEVPQRILRIMCRLEELGLAGRCLTLTPRPATEAELLTCHSAEYVGHLRATEKMKTRELHRESSNFDSIYICPSTFACAQLATGAACRLVEAVLSGEVLNGAAVVRPPGHHAEQDAACGFCFFNSVAVAARHAQTISGHALRILIVDWDVHHGNGTQHMFEDDPSVLYVSLHRYDHGTFFPMGDEGASSQIGRAAGTGFTVNVAWNGPRMGDADYLAAWHRLVLPIAYEFNPELVLVSAGFDAARGDPLGGCQVSPEGYAHLTHLLMGLASGRIILILEGGYNLTSISESMAACTRSLLGDPPPLLTLPRPPLSGALASITETIQVHRRYWRSLRVMKVEDREGPSSSKLVTKKAPQPAKPRLAERMTTREKKVLEAGMGKVTSASFGEESTPGQTNSETAVVALTQDQPSEAATGGATLAQTISEAAIGGAMLGQTTSEEAVGGATPDQTTSEETVGGAILDQTTSEDAVGGATLGQTTSEEAVGGATLAQTTSEAAMEGATLDQTTSEEAPGGTELIQTPLASSTDHQTPPTSPVQGTTPQISPSTLIGSLRTLELGSESQGASESQAPGEENLLGEAAGGQDMADSMLMQGSRGLTDQAIFYAVTPLPWCPHLVAVCPIPAAGLDVTQPCGDCGTIQENWVCLSCYQVYCGRYINGHMLQHHGNSGHPLVLSYIDLSAWCYYCQAYVHHQALLDVKNIAHQNKFGEDMPHP

SEQ ID NO: 18 - protein, 377 aa, Homo sapiens
MEEPEEPADSGQSLVPVYIYSPEYVSMCDSLAKIPKRASMVHSLIEAYALHKQMRIVKPKVASMEEMATFHTDAYLQHLQKVSQEGDDDHPDSIEYGLGYDCPATEGIFDYAAAIGGATITAAQCLIDGMCKVAINWSGGWHHAKKDEASGFCYLNDAVLGILRLRRKFERILYVDLDLHHGDGVEDAFSFTSKVMTVSLHKFSPGFFPGTGDVSDVGLGKGRYYSVNVPIQDGIQDEKYYQICESVLKEVYQAFNPKAVVLQLGADTIAGDPMCSFNMTPVGIGKCLKYILQWQLATLILGGGGYNLANTARCWTYLTGVILGKTLSSEIPDHEFFTAYGPDYVLEITPSCRPDRNEPHRIQQILNYIKGNLKHVV

SEQ ID NO: 19 - DNA, 21 bp, synthetic construct
  source 1..21: mol_type = other DNA
cggttaggttgcttcaatcta

SEQ ID NO: 20 - DNA, 21 bp, synthetic construct
  source 1..21: mol_type = other DNA
cagtctcaccaatttcagaaa

SEQ ID NO: 21 - DNA, 21 bp, synthetic construct
  source 1..21: mol_type = other DNA
caagagtcttaatgccttcaa

SEQ ID NO: 22 - DNA, 21 bp, synthetic construct
  source 1..21: mol_type = other DNA
gccaaagatgacttccctctt

SEQ ID NO: 23 - DNA, 21 bp, synthetic construct
  source 1..21: mol_type = other DNA
catcccatcctgaatatcctt

SEQ ID NO: 24 - DNA, 21 bp, synthetic construct
  source 1..21: mol_type = other DNA
gcgtattctctacgtggattt

SEQ ID NO: 25 - DNA, 7086 bp, synthetic construct
  source 1..7086: mol_type = other DNA
ttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccaccggagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcacgaaggctgtagacaaatactgggacagctacaaccatcccttcagacaggatcagaagaacttacatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatcacgagactagcctcgagcggccgcccccttcaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggcaacaagatgaagagcaccaactcgagttggtgctcttcatcttgttgtttttgaattctcgacctcgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggccgcggctcgaggggg

SEQ ID NO: 26 - DNA, 7518 bp, synthetic construct
  source 1..7518: mol_type = other DNA
cccgggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccaccggagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgccgtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtacgtatagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccagagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaggtaccggcggttaggttgcttcaatctactcgagtagattgaagcaacctaaccgtttttgaattctagatcttgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggcgccggctcgaggggg

SEQ ID NO: 27 - DNA, 7518 bp, synthetic construct
  source 1..7518: mol_type = other DNA
cccgggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccaccggagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgccgtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtacgtatagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccagagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaggtaccggcagtctcaccaatttcagaaactcgagtttctgaaattggtgagactgtttttgaattctagatcttgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggcgccggctcgaggggg

SEQ ID NO: 28 - DNA, 7518 bp, synthetic construct
  source 1..7518: mol_type = other DNA
cccgggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccaccggagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgccgtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtacgtatagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccagagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaggtaccggcaagagtcttaatgccttcaactcgagttgaaggcattaagactcttgtttttgaattctagatcttgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggcgccggctcgaggggg

SEQ ID NO: 29 - DNA, 7516 bp, synthetic construct
  source 1..7516: mol_type = other DNA
cccgggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccaccggagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgccgtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtacgtatagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccagagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaggtaccgggccaaagatgacttccctcttctcgagaagagggaagtcatctttggctttttgaattctagatcttgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggcgccggctcgaggg

SEQ ID NO: 30 - DNA, 7518 bp, synthetic construct
  source 1..7518: mol_type = other DNA
cccgggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccaccggagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgccgtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtacgtatagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccagagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaggtaccggcatcccatcctgaatatccttctcgagaaggatattcaggatgggatgtttttgaattctagatcttgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggcgccggctcgaggggg

SEQ ID NO: 31 - DNA, 7518 bp, synthetic construct
  source 1..7518: mol_type = other DNA
cccgggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccaccggagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgccgtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtacgtatagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccagagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaggtaccgggcgtattctctacgtggatttctcgagaaatccacgtagagaatacgctttttgaattctagatcttgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggcgccggctcgaggggg

SEQ ID NO: 32 - DNA, 39 bp, synthetic construct
  source 1..39: mol_type = other DNA
atggtgagcaagggcgaggagctgttcaccggggtggtg

SEQ ID NO: 33 - DNA, 33 bp, synthetic construct
  source 1..33: mol_type = other DNA
atggtgagcaagggcgaggagctgttggtggtg

SEQ ID NO: 34 - DNA, 29 bp, synthetic construct
  source 1..29: mol_type = other DNA
atggtgagcaagggcgaggccgggtggtg

SEQ ID NO: 35 - DNA, 41 bp, synthetic construct
  source 1..41: mol_type = other DNA
atggtgagcaagggcgaggagctgttcaccccggggtggtg

SEQ ID NO: 36 - DNA, 30 bp, synthetic construct
  source 1..30: mol_type = other DNA
atggtgagcaagggcgaggagggggtggtg

SEQ ID NO: 37 - DNA, 42 bp, synthetic construct
  source 1..42: mol_type = other DNA
atggtgagcaagggcgaggagctgttcaccgggcgggtggtg

SEQ ID NO: 38-39 - skipped (2 entries)

SEQ ID NO: 40 - DNA, 19 bp, synthetic construct
  source 1..19: mol_type = other DNA
atggtgagcaaggatggtg

SEQ ID NO: 41 - DNA, 40 bp, synthetic construct
  source 1..40: mol_type = other DNA
atggtgagcaagggcgaggagctgttccaccggggtggtg

SEQ ID NO: 42 - DNA, 39 bp, synthetic construct; identical to SEQ ID NO: 32
  source 1..39: mol_type = other DNA

SEQ ID NO: 43 - DNA, 41 bp, synthetic construct; identical to SEQ ID NO: 35
  source 1..41: mol_type = other DNA

SEQ ID NO: 44 - DNA, 42 bp, synthetic construct
  source 1..42: mol_type = other DNA
atggtgagcaagggcgaggagctgttcaccgggggggtggtg

SEQ ID NO: 45 - DNA, 40 bp, synthetic construct
  source 1..40: mol_type = other DNA
atggtgagcaagggcgaggagctgttacaccggggtggtg

SEQ ID NO: 46 - DNA, 44 bp, synthetic construct
  source 1..44: mol_type = other DNA
atggtgagcaagggcgaggagctgttcacccccggggggtggtg

SEQ ID NO: 47 - DNA, 21 bp, synthetic construct
  source 1..21: mol_type = other DNA
atggtgagcaagggcgaggtg

SEQ ID NO: 48 - DNA, 19 bp, synthetic construct
  source 1..19: mol_type = other DNA
atggtgagcaagggtggtg

SEQ ID NO: 49 - DNA, 10 bp, synthetic construct
  source 1..10: mol_type = other DNA
atgggtggtg

SEQ ID NO: 50 - DNA, 29 bp, synthetic construct
  source 1..29: mol_type = other DNA
atggtgagcaagggcgaggagctgtggtg

SEQ ID NO: 51 - DNA, 36 bp, synthetic construct
  source 1..36: mol_type = other DNA
atggtgagcaagggcgaggagctgttcagggtggtg

SEQ ID NO: 52 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 53 - protein, 1368 aa, Streptococcus pyogenes
MDKKYSIGLDIGTNSVGWAVITDDYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRLNSEITKAPLSASMIKRYDEHHQDLTLLKALVRQQLPEKYKEIFFDQSKNGYAGYIDGGASQEEFYKFIKPILEKMDGTEELLAKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFIKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGLTIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 54 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 55 - DNA, 4107 bp, synthetic construct
  source 1..4107: mol_type = other DNA
atggataaaaagtattctattggtttagacatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatcacattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgtgagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccagagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaagagtacagatctaccaaggaggtgctagacgcgacactgattcaccaatccatcacgggattatatgaaactcggatagatttgtcacagcttgggggtgactga

SEQ ID NO: 56 - protein, 108 aa, synthetic construct
MSEGVQVKRVLEKSPGKLLVKMPFQTSPGGKAEGGGATTSTQVMVIKRPGRKRKAEADPQAIPKKRGRKPGSVVAAAAAEAKKKAVKESSIRSVQETVLPIKKRKTRE

SEQ ID NO: 57 - DNA, 327 bp, synthetic construct
  source 1..327: mol_type = other DNA
atgtcggagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagacagtactccccatcaagaagcgcaagacccgggagtaa

SEQ ID NO: 58 - DNA, 537 bp, synthetic construct
  source 1..537: mol_type = other DNA
atgcggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctcgggaggtggttcgggaggtggttcggagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagacagtactccccatcaagaagcgcaagacccgggagtaa

SEQ ID NO: 59 - protein, 178 aa, synthetic construct
MRTLVTFKDVFVDFTREEWKLLDTAQQIVYRNVMLENYKNLVSLGYQLTKPDVILRLEKGEEPSGGGSGGGSEGVQVKRVLEKSPGKLLVKMPFQTSPGGKAEGGGATTSTQVMVIKRPGRKRKAEADPQAIPKKRGRKPGSVVAAAAAEAKKKAVKESSIRSVQETVLPIKKRKTRE

SEQ ID NO: 60 - protein, 311 aa, synthetic construct
PSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 61 - DNA, 933 bp, synthetic construct
  source 1..933: mol_type = other DNA
ccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtgttcatgaatgagaaagaggacatcttatggtgcactgaaatggaaagggtatttggtttcccagtccactatactgacgtgtccaacatgagccgcttggcgaggcagagactgctgggccggtcatggagcgtgccagtcatccgccacctcttcgctccgctgaaggagtattttgcgtgtgtg

SEQ ID NO: 62 - protein, 1702 aa, synthetic construct
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKLEGGGGSGSPSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFAC

SEQ ID NO: 63 - DNA, 5109 bp, synthetic construct
  source 1..5109: mol_type = other DNA
gacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggacaggccaaaaagaaaaagctcgagggcggaggcgggagcggatccccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtgttcatgaatgagaaagaggacatcttatggtgcactgaaatggaaagggtatttggtttcccagtccactatactgacgtgtccaacatgagccgcttggcgaggcagagactgctgggccggtcatggagcgtgccagtcatccgccacctcttcgctccgctgaaggagtattttgcgtgtgtg

SEQ ID NO: 64 - protein, 1688 aa, synthetic construct
MAPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLAGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNLKGSPEDNEQKQLFVEQHKHYLDEIIEQISEFSKRVILADANLDKVLSAYNKHRDKPIREQAENIIHLFTLTNLGAPAAFKYFDTTIDRKEYRSTKEVLDATLIHQSITGLYETRIDLSQLGGDGSPKKKVLEGGGGSGSPSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 65 - DNA, 5208 bp, synthetic construct
  source 1..5208: mol_type = other DNA
atggactataaggaccacgacggagactacaaggatcatgatattgattacaaagacgatgacgataagatggccccaaagaagaagcggaaggtcggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgtgagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccagagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaagagtacagatctaccaaggaggtgctagacgcgacactgattcaccaatccatcacgggattatatgaaactcggatagatttgtcacagcttgggggtgacggatcccccaagaagaagaggaaagtcctcgagggcggaggcgggagcggatccccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtgttcatgaatgagaaagaggacatcttatggtgcactgaaatggaaagggtatttggtttcccagtccactatactgacgtgtccaacatgagccgcttggcgaggcagagactgctgggccggtcatggagcgtgccagtcatccgccacctcttcgctccgctgaaggagtattttgcgtgtgtg